(19) United States
(12) Patent Application Publication
(10) Pub. No.: US 2025/0340568 A1
(43) Pub. Date: Nov. 6, 2025

(54) SOLID FORMS OF A MACROCYCLIC COMPOUNDS AS CFTR MODULATORS AND THEIR PREPARATION

(71) Applicant: Vertex Pharmaceuticals Incorporated, Boston, MA (US)

(72) Inventors: Minson BAEK, San Diego, CA (US); Kevin GAGNON, Acton, MA (US); Ales MEDEK, Winchester, MA (US); Andrey PERESYPKIN, Newton, MA (US); Stefanie ROEPER, Medford, MA (US); Yi SHI, Natick, MA (US); Muna SHRESTHA, Belmont, MA (US); David A. SIESEL, San Diego, CA (US)

(21) Appl. No.: 18/864,468

(22) PCT Filed: May 15, 2023
(86) PCT No.: PCT/US2023/022263

§ 371 Date: Nov. 8, 2024

Related U.S. Application Data
(60) Provisional application No. 63/342,392, filed on May 16, 2022.
(60) Provisional application No. 63/342,408, filed on May 16, 2022.

(51) Int. Cl. C07D 498/06; C07D 213/81; C07D 498/16

(52) U.S. Cl. CPC C07D 498/06; C07D 213/81; C07D 498/16

(57) ABSTRACT

Processes and methods of preparing Compound I are disclosed. Crystalline forms of Compound I, pharmaceutically acceptable salts, solvates, hydrates, and cocrystals thereof, pharmaceutical compositions comprising the same, methods of treating cystic fibrosis using the same, and methods for making the same are also disclosed.

This application claims the benefit of U.S. Provisional Application No. 63/342,392, filed on May 16, 2022, and U.S. Provisional Application No. 63/342,408, filed on May 16, 2022, the contents of which are incorporated by reference in their entirety.

Disclosed herein are crystalline and amorphous solid forms of a Cystic Fibrosis Transmembrane Conductance Regulator (CFTR) modulator, pharmaceutical compositions thereof, methods of treating cystic fibrosis with any of the foregoing, and processes for making the crystalline and amorphous forms. Further disclosed herein are processes and methods of preparing CFTR modulators.

Cystic fibrosis (CF) is a recessive genetic disease that affects approximately 88,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S. et al. (1990) Proc. Natl. Acad. Sci. USA 87:8447-8451). To date, greater than 2000 mutations in the CF gene have been identified; currently, the CFTR2 database contains information on at least 322 of these identified mutations, with sufficient evidence to define at least 281 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC (epithelial sodium channel) and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺co-transporter, Na⁺—K⁺-ATPase pump and the basolateral membrane K⁺channels on the basolateral surface and CFTR on the luminal side coordinate the secretion of chloride. Because water is probably never actively transported itself, its flow across epithelia depends on tiny transepithelial osmotic gradients generated by the bulk flow of sodium and chloride.

A number of CFTR modulators have recently been identified. These modulators can be characterized as, for example, potentiators, correctors, potentiator enhancers/co-potentiators, amplifiers, readthrough agents, and nucleic acid therapies. CFTR modulators that increase the channel gating activity of mutant and wild-type CFTR at the epithelial cell surface are known as potentiators. Correctors improve faulty protein processing and resulting trafficking to the epithelial surface. Ghelani and Schneider-Futschik (2020) ACS Pharmacol. Transl. Sci. 3:4-10. There are three CFTR correctors approved by the U.S. FDA for treatment of cystic fibrosis. However, monotherapy with some CFTR correctors has not been found to be effective enough and as a result combination therapy with a potentiator is needed to enhance CFTR activity. There is currently only one CFTR potentiator that is approved for the treatment of cystic fibrosis. Thus, although the treatment of cystic fibrosis has been transformed by these new small molecule CFTR modulators, new and better modulators are needed to prevent disease progression, reduce the severity of the cystic fibrosis and other CFTR-mediated diseases, and to treat the more severe forms of these diseases.

Thus, one aspect of the disclosure provides solid forms of a CFTR-modulating compound, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I), and pharmaceutically acceptable salts thereof. Compound I can be depicted as having the following structure:

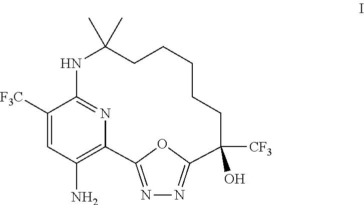 [SMILES: CC1(C)CCCCC[C@](O)(C(F)(F)F)C2=NN=C(O2)c2nc(c(C(F)(F)F)cc2N)N1]

A further aspect of the disclosure provides methods of preparing Compound I, stereoisomers of Compound I, deuterated derivatives of Compound I and its stereoisomers, and pharmaceutically acceptable salts of any of the foregoing.

Compound I was first described in PCT International Application No. PCT/US2021/072475, which published as WO 2022/109573, and which isincorporated herein by reference in its entirety. Compound I was disclosed in WO 2022/109573 as crystalline Form A (neat).

Crystalline forms are of interest in the pharmaceutical industry, where the control of the crystalline form(s) of the active ingredient may be desirable or even required. Reproducible processes for producing a compound with a particular crystalline form in high purity may be desirable for compounds intended to be used in pharmaceuticals, as different crystalline forms may possess different properties. For example, different crystalline forms may possess different chemical, physical, and/or pharmaceutical properties. In some embodiments, one or more crystalline forms disclosed herein may exhibit a higher level of purity, chemical stability, and/or physical stability. Certain crystalline forms (e.g., crystalline free form, crystalline salt, crystalline salt solvate, and crystalline salt hydrate forms of Compound I (collectively referred to as “crystalline forms”)) may exhibit lower hygroscopicity. Thus, the crystalline forms of this disclosure may provide advantages during drug substance manufacturing, storage, and handling. Thus, pharmaceutically acceptable crystalline forms of Compound I may be particularly useful for the production of drugs for the treatment of CFTR-mediated diseases.

Amorphous forms of therapeutic compounds may also be of interest in the pharmaceutical industry, where crystalline forms are not especially bioavailable. Some amorphous forms may improve bioavailability and thus allow for administration of reduced dosages. For some compounds, amorphous forms provide the most biologically accessible form of the therapeutic.

In some embodiments, the crystalline form of Compound I is Compound I methanol solvate (wet). In some embodiments, the crystalline form of Compound I is Compound I methanol solvate (dry). In some embodiments, the crystalline form of Compound I is Compound I p-toluene sulfonic acid.

In some embodiments, the solid form of Compound I is an amorphous form. In some embodiments, the solid amorphous form of Compound I is Compound I neat amorphous form.

Another aspect of the invention provides pharmaceutical compositions comprising at least one solid form chosen from solid forms of Compound I, pharmaceutically acceptable salts thereof, and deuterated derivives of any of the foregoing disclosed herein, which compositions may further include at least one additional active pharmaceutical ingredient and/or at least one carrier.

In certain embodiments, the pharmaceutical compositions of the invention comprise Compound I in any of the pharmaceutically acceptable solid forms disclosed herein. In some embodiments, compositions comprising Compound I in any of the pharmaceutically acceptable crystalline forms disclosed herein may optionally further comprise at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof.

Another aspect of the invention provides methods of treating the CFTR-mediated disease cystic fibrosis comprising administering Compound I in any of the pharmaceutically acceptable solid forms disclosed herein, optionally as part of a pharmaceutical composition comprising at least one additional component (such as a carrier or additional active agent), to a subject in need thereof. In some embodiments, methods of treating the CFTR-mediated disease cystic fibrosis comprise administering Compound I in any of the pharmaceutically acceptable solid forms disclosed herein, and optionally further administering one or more additional CFTR modulating agents selected from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), N-[2,4-bis(1,1-dimethylethyl)-5-hydroxyphenyl]-1,4-dihydro-4-oxoquinoline-3-carboxamide (Compound III) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound III-d), 3-(6-(1-(2,2-difluorobenzo[d][1,3]dioxol-5-yl)cyclopropane carboxamido)-3-methylpyridin-2-yl)benzoic acid (Compound IV), N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound V), N-(benzenesulfonyl)-6-[3-[2-[1-(trifluoromethyl) cyclopropyl]ethoxy]pyrazol-1-yl]-2-[(4S)-2,2,4-trimethylpyrrolidin-1-yl]pyridine-3-carboxamide (Compound VI), (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione (Compound VII), (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound VIII); N-(benzenesulfonyl)-6-(3-fluoro-5-isobutoxy-phenyl)-2-[(4S)-2,2,4-trimethylpyrrolidin-1-yl]pyridine-3-carboxamide (Compound IX), and N′-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X).

A further aspect of the disclosure provides processes of making the solid forms of Compound I disclosed herein.

Another aspect of the invention provides solid forms of Compound I, pharmaceutically acceptable salts thereof, and deuterated derivives of any of the foregoing disclosed herein, for use in any of the methods described herein.

### BRIEF DESCRIPTION OF THE FIGURES

FIG. 1 provides an X-ray power diffraction (XRPD) pattern of Compound I neat Form A.

FIG. 2 provides a differential scanning calorimetry (DSC) analysis of Compound I neat Form A.

FIG. 3 provides an XRPD pattern of crystalline Compound I methanol solvate (wet).

FIG. 4 provides a ¹³C SSNMR spectrum of crystalline Compound I methanol solvate (wet).

FIG. 5 provides a ¹⁹F SSNMR spectrum of crystalline Compound I methanol solvate (wet).

FIG. 6 provides an XRPD pattern of crystalline Compound I methanol solvate (dry).

FIG. 7 provides a TGA curve for crystalline Compound I methanol solvate (dry).

FIG. 8 provides a DSC analysis of crystalline Compound I methanol solvate (dry).

FIG. 9 provides an XRPD pattern of crystalline Compound I p-toluene sulfonic acid.

FIG. 10 provides a DSC analysis of crystalline Compound I p-toluene sulfonic acid.

FIG. 11 provides a ¹³C SSNMR spectrum of crystalline Compound I p-toluene sulfonic acid.

FIG. 12 provides a ¹⁹F SSNMR spectrum of crystalline Compound I p-toluene sulfonic acid.

FIG. 13 provides an XRPD pattern of neat amorphous Compound I.

FIG. 14 provides a TGA curve for neat amorphous Compound I.

FIG. 15 provides a DSC analysis of neat amorphous Compound I.

### DEFINITIONS

“Compound I” as used throughout this disclosure refers to (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, which can be depicted as having the following structure:

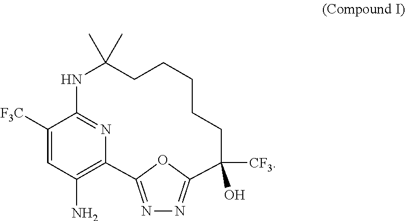 [SMILES: CC1(C)CCCCC[C@](O)(C(F)(F)F)C2=NN=C(O2)c2nc(c(C(F)(F)F)cc2N)N1]

Compound I may be a racemic mixture or an enantioenriched (e.g., >90% ee, >95% ee, >98% ee) mixture of isomers. Compound I may be in the form of a pharmaceutically acceptable salt, solvate, and/or hydrate. Compound I and methods for making and using Compound I, stereoisomers of Compound I, deuterated derivatives of Compound I and its stereoisomers, and pharmaceutically acceptable salts of any of the foregoing are disclosed in WO 2022/109573, incorporated herein by reference.

“Compound II” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

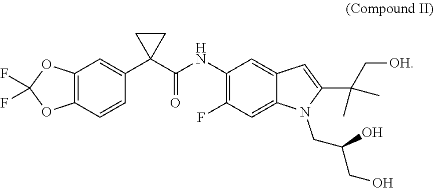 [SMILES: CC(C)(CO)c1cc2cc(NC(=O)C3(c4ccc5c(c4)OC(F)(F)O5)CC3)c(F)cc2n1C[C@@H](O)CO]

Compound II may be in the form of a pharmaceutically acceptable salt. Compound II and methods of making and using Compound II are disclosed in WO 2010/053471, WO 2011/119984, WO 2011/133751, WO 2011/133951, and WO 2015/160787, each incorporated herein by reference.

“Compound III” as used throughout this disclosure refers to N-(5-hydroxy-2,4-di-tert-butyl-phenyl)-4-oxo-1H-quinoline-3-carboxamide which is depicted by the structure:

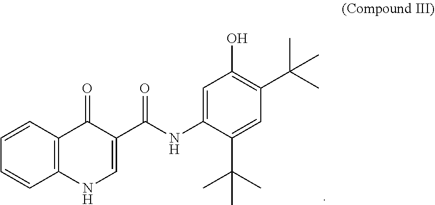 [SMILES: CC(C)(C)c1cc(C(C)(C)C)c(NC(=O)c2c[nH]c3ccccc3c2=O)cc1O]

Compound III may also be in the form of a pharmaceutically acceptable salt. Compound III and methods of making and using Compound III are disclosed in WO 2006/002421, WO 2007/079139, WO 2010/108162, and WO 2010/019239, each incorporated herein by reference.

In some embodiments, a deuterated derivative of Compound III (Compound III-d) is employed in the compositions and methods disclosed herein. A chemical name for Compound III-d is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

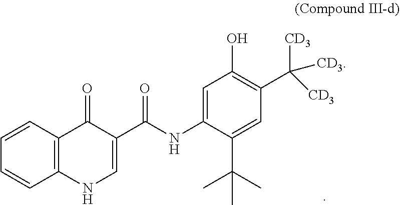 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Compound III-d may be in the form of a pharmaceutically acceptable salt. Compound III-d and methods of making and using Compound III-d are disclosed in WO 2012/158885, WO 2014/078842, and U.S. Pat. No. 8,865,902, incorporated herein by reference.

“Compound IV” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

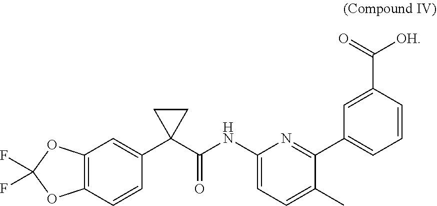 [SMILES: Cc1ccc(NC(=O)C2(c3ccc4c(c3)OC(F)(F)O4)CC2)nc1-c1cccc(C(=O)O)c1]

Compound IV may be in the form of a pharmaceutically acceptable salt. Compound IV and methods of making and using Compound IV are disclosed in WO 2007/056341, WO 2009/073757, and WO 2009/076142, incorporated herein by reference.

“Compound V” as used herein, refers to N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide, which is depicted by the chemical structure:

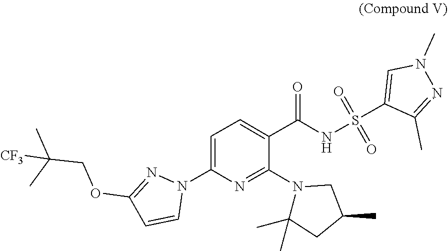 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

Compound V may be in the form of a pharmaceutically acceptable salt. Compound V and methods of making and using Compound V are disclosed in WO 2018/107100 and WO 2019/113476, incorporated herein by reference.

“Compound VI” as used herein, refers to N-(benzenesulfonyl)-6-[3-[2-[1-(trifluoromethyl) cyclopropyl]ethoxy]pyrazol-1-yl]-2-[(4S)-2,2,4-trimethylpyrrolidin-1-yl]pyridine-3-carboxamide, which is depicted by the chemical structure:

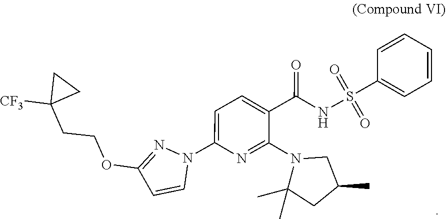 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

Compound VI may be in the form of a pharmaceutically acceptable salt. Compound VI and methods of making and using Compound VI are disclosed in WO 2018/064632, incorporated herein by reference.

“Compound VII” as used herein, refers to (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione, which is depicted by the chemical structure:

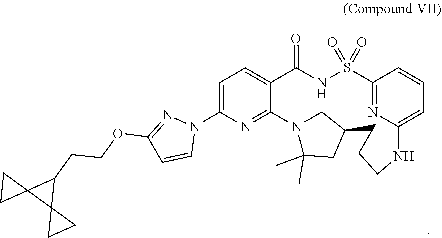 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

Compound VII may be in the form of a pharmaceutically acceptable salt. Compound VII and methods of making and using Compound VII are disclosed in WO 2019/161078, WO 2020/102346, and PCT Application No. PCT/US2020/046116, incorporated herein by reference.

“Compound VIII” as used herein, refers to (11R)-6-(2,6-dimethylphenyl)-11-(2-methylpropyl)-12-{spiro[2.3]hexan-5-yl}-9-oxa-2⁶-thia-3,5,12,19-tetraazatricyclo[12.3.1.14,8]nonadeca-1(17),4(19),5,7,14(18),15-hexaene-2,2,13-trione, which is depicted by the chemical structure:

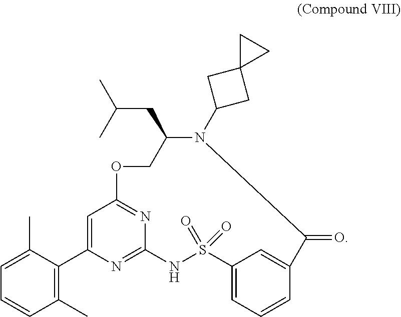 [SMILES: Cc1cccc(C)c1-c1cc2nc(n1)NS(=O)(=O)c1cccc(c1)C(=O)N(C1CC3(CC3)C1)[C@H](CC(C)C)CO2]

Compound VIII may be in the form of a pharmaceutically acceptable salt. Compound VIII and methods of making and using Compound VIII are disclosed in WO 2020/206080, incorporated herein by reference.

“Compound IX” as used herein, refers to N-(benzenesulfonyl)-6-(3-fluoro-5-isobutoxy-phenyl)-2-[(4S)-2,2,4-trimethylpyrrolidin-1-yl]pyridine-3-carboxamide, which is depicted by the chemical structure:

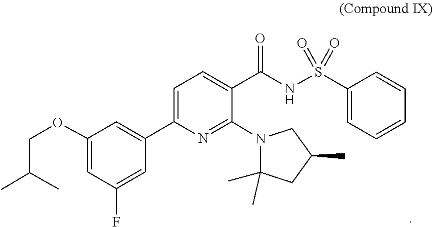 [SMILES: CC(C)COc1cc(F)cc(-c2ccc(C(=O)NS(=O)(=O)c3ccccc3)c(N3C[C@@H](C)CC3(C)C)n2)c1]

Compound IX may be in the form of a pharmaceutically acceptable salt. Compound IX and methods of making and using Compound IX are disclosed in WO 2016/057572, incorporated herein by reference.

“Compound X” as used herein, refers to N′-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, which is depicted by the chemical structure:

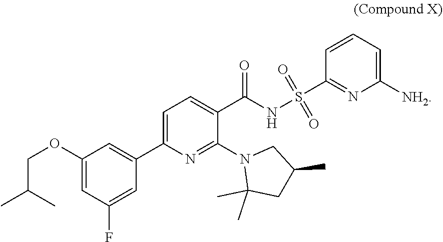 [SMILES: CC(C)COc1cc(F)cc(-c2ccc(C(=O)NS(=O)(=O)c3cccc(N)n3)c(N3C[C@@H](C)CC3(C)C)n2)c1]

Compound X may be in the form of a pharmaceutically acceptable salt. Compound X and methods of making and using Compound X are disclosed in WO 2016/057572, incorporated herein by reference.

As used herein, “CFTR” means cystic fibrosis transmembrane conductance regulator.

As used herein, the terms “CFTR modulator” and “CFTR modulating compound” interchangeably refer to a compound that directly or indirectly increases the activity of CFTR. The increase in activity resulting from a CFTR modulator includes but is not limited to compounds that correct, potentiate, stabilize, and/or amplify CFTR.

As used herein, the term “CFTR corrector” refers to a compound that facilitates the processing and trafficking of CFTR to increase the amount of CFTR at the cell surface.

As used herein, the term “CFTR potentiator” refers to a compound that increases the channel activity of CFTR protein located at the cell surface, resulting in enhanced ion transport.

As used herein, the term “CFTR potentiator enhancer,” “CFTR potentiation enhancer,” and “CFTR co-potentiator” are used interchangeably and refer to a compound that enhances CFTR potentiation.

As used herein, the term “active pharmaceutical ingredient” (“API”) or “therapeutic agent” refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or one or more of its symptoms or lessening the severity of CF or one or more of its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrently with, or subsequent to each other.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. In general, a genetic defect or mutation, or a change in the nucleotides in a gene, results in a mutation in the CFTR protein translated from that gene, or a frame shift(s).

As used herein, the term “F508del” refers to a mutant CFTR protein which is lacking the amino acid phenylalanine at position 508, or to a mutant CFTR gene which encodes for a CFTR protein lacking the amino acid phenylalanine at position 508.

As used herein, the term “unsaturated” means that a moiety has one or more units of unsaturation.

As used herein, the term “alkyl” means a saturated or partially saturated, branched, or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more adjacent carbon atoms is interrupted by a double (alkenyl) or triple (alkynyl) bond. Alkyl groups may be substituted or unsubstituted.

The term “aliphatic” or “aliphatic group,” as used herein, means a straight-chain (i.e., unbranched) or branched, substituted, or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic,” “carbocycle,” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃₋₈ hydrocarbon or bicyclic or tricyclic C₈₋₁₄ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof, such as (cycloalkyl)alkyl, (cycloalkenyl)alkyl, and (cycloalkyl)alkenyl. Suitable cycloaliphatic groups include cycloalkyl, bicyclic cycloalkyl (e.g., decalin), bridged bicycloalkyl such as norbomyl or [2.2.2]bicyclo-octyl, and bridged tricyclic such as adamantyl.

As used herein, the term “halogen” or “halo” means F, Cl, Br, or I.

As used herein, term “alkoxy” refers to an alkyl or cycloalkyl covalently bonded to an oxygen atom. Alkoxy groups may be substituted or unsubstituted.

As used herein, “cycloalkyl group” refers to a cyclic, non-aromatic hydrocarbon group containing 3-12 carbons in a ring (such as, for example, 3-10 carbons). Cycloalkyl groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, spiro[2.2]pentane, and dispiro[2.0.2.1]heptane. Cycloalkyl groups may be substituted or unsubstituted.

The term “heteroatom” means one or more of oxygen, sulfur, nitrogen, phosphorus, or silicon (including any oxidized form of nitrogen, sulfur, phosphorus, or silicon; the quaternized form of any basic nitrogen; and a substitutable nitrogen of a heterocyclic ring, for example, N (as in 3,4-dihydro-2H-pyrrolyl), NH (as in pyrrolidinyl) or NR⁺(as in N-substituted pyrrolidinyl)).

The term “heterocyclyl,” “heterocycle,” or “heterocyclic” as used herein means non-aromatic monocyclic, bicyclic, tricyclic, polycyclic, bridged, fused, and spiro ring systems, including mono spiro and dispiro ring systems, in which one or more ring members is an independently chosen heteroatom. In some embodiments, the “heterocycle,” “heterocyclyl,” or “heterocyclic” group has three to fourteen ring members in which one or more ring members is a heteroatom independently chosen from oxygen, sulfur, nitrogen, and phosphorus, and each ring in the system contains three to seven ring members.

As used herein, the term “aryl” is a functional group or substituent derived from an aromatic ring and encompasses monocyclic aromatic rings and bicyclic, tricyclic, and fused ring systems wherein at least one ring in the system is aromatic. An aryl group may be optionally substituted with one or more substituents. Non-limiting examples of aryl groups include phenyl, naphthyl, and 1,2,3,4-tetrahydronaphthalenyl.

As used herein, the term “heteroaryl” refers to an aromatic ring comprising at least one ring atom that is a heteroatom, such as O, N, or S. Heteroaryl groups encompass monocyclic, bicyclic, and tricyclic ring systems having a total of five to fourteen ring members, wherein at least one ring in the system is aromatic, at least one ring in the system contains one or more heteroatoms, and wherein each ring in the system contains three to seven ring members. Non-limiting examples of heteroaryl rings include pyridine, quinoline, indole, and indoline. A heteroaryl group may be optionally substituted with one or more substituents. In certain embodiments, the term “heteroaryl ring” encompasses heteroaryl rings with various oxidation states, such as heteroaryl rings containing N-oxides and sulfoxides. Non-limiting examples of such heteroaryl rings include pyrimidine N-oxides, quinoline N-oxides, thiophene S-oxides, and pyrimidine N-oxides.

“Tert” and “t-” are used interchangeably and mean tertiary.

The term “stable,” as used herein, refers to compounds that are not substantially altered when subjected to conditions to allow for their production, detection, and preferably their recovery, purification, and use for one or more of the purposes disclosed herein.

“Selected from” and “chosen from” are used interchangeably herein.

As used herein, the term “solvent” refers to any liquid in which the product is at least partially soluble (solubility of product >1 g/L).

Non-limiting examples of suitable solvents that may be used in this disclosure include, for example, water (H₂O), methanol (MeOH), methylene chloride or dichloromethane (DCM; CH₂Cl₂), acetonitrile (MeCN; CH₃CN), N,N-dimethylformamide (DMF), dimethylsulfoxide (DMSO), methyl acetate (MeOAc), ethyl acetate (EtOAc), isopropyl acetate (IPAc), tert-butyl acetate (t-BuOAc), isopropyl alcohol (IPA), tetrahydrofuran (THF), 2-methyl tetrahydrofuran (2-MeTHF), methyl ethyl ketone (MEK), tert-butanol, diethyl ether (Et₂O), methyl tert-butyl ether (MTBE), 1,4-dioxane, and N-methylpyrrolidone (NMP).

The term “protecting group,” as used herein, refers to any chemical group introduced into a molecule by chemical modification of a functional group to obtain chemoselectivity in a subsequent chemical reaction.

Methods of adding (a process generally referred to as “protecting”) and removing (process generally referred to as “deprotecting”) protecting groups are well-known in the art and available, for example, in P. J. Kocienski, Protecting Groups, 3^{rd} edition (Thieme, 2005), and in Greene and Wuts, Protective Groups in Organic Synthesis, 4th edition (John Wiley & Sons, New York, 2007), both of which are hereby incorporated by reference in their entirety.

Non-limiting examples of useful protecting groups for amines that may be used in this disclosure include monovalent protecting groups, for example, t-butyloxycarbonyl (Boc), benzyl (Bn), β-methoxyethoxytrityl (MEM), tetrahydropyranyl (THP), 9-fluorenylmethyloxycarbonyl (Fmoc), benzyloxycarbonyl (Cbz), formyl, acetyl (Ac), trifluoroacetyl (TFA), trityl (Tr), and p-toluenesulfonyl (Ts); and divalent protecting groups, for example, benzylidene, 4,5-diphenyl-3-oxazolin-2-one, N-phthalimide, N-dichlorophthalimide, N-tetrachlorophthalimide, N-4-nitrophthalimide, N-thiodiglycoloyl amine, N-dithiasuccinimide, N-2,3-diphenylmaleimide, N-2,3-dimethylmaleimide, N-2,5-dimethylpyrrole, N-2,5-bis(triisopropylsiloxy)pyrrole (BIPSOP), N-1,1,4,4-tetramethyldisilylazacyclopentane (STABASE), N-1,1,3,3-tetramethyl-1,3-disilaisoindoline (Benzostabase, BSB), N-diphenylsilyldiethylene, N-5-substituted 1,3-dimethyl-1,3,5-triazacyclohexan-2-one, N-5-substituted 1,3-dibenzyl-1,3,5-triazacyclohexan-2-one, 1-substituted 3,5-dinitro-4-pyridone, and 1,3,5-dioxazine.

Non-limiting examples of useful protecting groups for alcohols that may be used in this disclosure include, for example, acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS).

Non-limiting examples of useful protecting groups for carboxylic acids that may be used in this disclosure include, for example, methyl or ethyl esters, substituted alkyl esters such as 9-fluorenylmethyl, methoxymethyl (MOM), methylthiomethyl (MTM), tetrahydropyranyl (THP), tetrahydrofuranyl, β-methoxyethoxymethyl (MEM), 2-(trimethylsilyl)ethoxymethyl (SEM), benzyloxymethyl (BOM), pivaloyloxymethyl (POM), phenylacetoxymethyl, and cyanomethyl, acetyl (Ac), phenacyl, substituted phenacyl esters, 2,2,2-trichloroethyl, 2-haloethyl, o-chloroalkyl, 2-(trimethylsilyl)ethyl, 2-methylthioethyl, t-butyl, 3-methyl-3-pentyl, dicyclopropylmethyl, cyclopentyl, cyclohexyl, allyl, methallyl, cinnamyl, phenyl (Ph), silyl esters, benzyl and substituted benzyl esters, 2,6-dialkylphenyl, and pentafluorophenyl (PFP).

Non-limiting examples of amine bases that may be used in this disclosure include, for example, 1,8-diazabicyclo[5.4.0]undec-7-ene (DBU), N-methylmorpholine (NMM), triethylamine (Et₃N; TEA), diisopropylethyl amine (i-Pr₂EtN; DIPEA), pyridine, 2,2,6,6-tetramethylpiperidine, 1,5,7-triazabicyclo[4.4.0]dec-5-ene (TBD), 7-methyl-1,5,7-triazabicyclo[4.4.0]dec-5-ene (MTBD), t-Bu-tetramethylguanidine, pyridine, 1,5-diazabicyclo[4.3.0]non-5-ene (DBN), and potassium bis(trimethylsilyl)amide (KHMDS).

Non-limiting examples of carbonate bases that may be used in this disclosure include, for example, sodium carbonate (Na₂CO₃), potassium carbonate (K₂CO₃), cesium carbonate (Cs₂CO₃), lithium carbonate (Li₂CO₃), sodium bicarbonate (NaHCO₃), and potassium bicarbonate (KHCO₃).

Non-limiting examples of alkoxide bases that may be used in this disclosure include, for example, t-AmOLi (lithium t-amylate), t-AmONa (sodium t-amylate), t-AmOK (potassium t-amylate), sodium tert-butoxide (NaOtBu), potassium tert-butoxide (KOtBu), and sodium methoxide (NaOMe; NaOCH₃).

Non-limiting examples of hydroxide bases that may be used in this disclosure include, for example, lithium hydroxide (LiOH), sodium hydroxide (NaOH), and potassium hydroxide.

Non-limiting examples of phosphate bases that may be used in this disclosure include, for example, sodium phosphate tribasic (Na₃PO₄), potassium phosphate tribasic (K₃PO₄), potassium phosphate dibasic (K₂HPO₄), and potassium phosphate monobasic (KH2PO₄).

Non-limiting examples of acids that may be used in this disclosure include, for example, trifluoroacetic acid (TFA), hydrochloric acid (HCl), methanesulfonic acid (MsOH), phosphoric acid (H₃PO₄), and sulfuric acid (H₂SO₄).

As used herein, the terms “reductant” and “reducing agent” are used interchangeably. As used herein, the terms “reducing conditions” and “reducing reaction conditions” are used interchangeably to refer to reaction conditions that involve the use of a reducing agent. Non-limiting examples of reducing agents and reducing conditions that may be used in this disclosure include, for example, H₂ and palladium on carbon; H₂ and palladium on alumina; sodium dithionite (Na₂S₂O₄); iron (Fe) and acetic acid (AcOH); and iron (Fe) and ammonium chloride (NH₄Cl).

As used herein, the term “sulfonyl chloride” means a compound in which a sulfonyl group (—SO₂—) is singly bonded to a chloride atom (e.g, RSO₂Cl). Non-limiting examples of sulfonyl chlorides include, for example, methanesulfonyl chloride (MeSO₂Cl), trifluoromethanesulfonyl chloride (F₃CSO₂Cl) benzenesulfonyl chloride (PhSO₂Cl), p-toluenesulfonyl chloride (4-MeC₆H₄SO₂Cl or TsCl), 2-nitrobenzylsulfonyl chloride (2-NO₂C₆H₄SO₂Cl or 2-NsCl), and 4-nitrobenzylsulfonyl chloride (4-NO₂C₆H₄SO₂Cl or 4-NsCl).

Non-limiting examples of suitable sulfonate esters —OSO₂R that may be used in this disclosure include, for example, methanesulfonyl (R=Me), trifluoromethanesulfonyl (R=CF₃) benzenesulfonyl (R=Ph), p-toluenesulfonyl (R=4-MeC₆H₄—), 2-nitrobenzylsulfonyl (R=2-NO₂C₆H₄—), and 4-nitrobenzylsulfonyl (R=4-NO₂C₆H₄—).

The term “compound,” when referring to a compound of this disclosure, refers to a collection of molecules having an identical chemical structure, except that there may be isotopic variation among the constituent atoms of the molecules.

Compounds described herein may optionally be substituted with one or more substituents, such as are illustrated generally above, or as exemplified by particular classes, subclasses, and species of the disclosure. It will be appreciated that the phrase “optionally substituted” is used interchangeably with the phrase “substituted or unsubstituted.” In general, the term “substituted,” whether preceded by the term “optionally” or not, indicates that at least one hydrogen of the “substituted” group is replaced with a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position. Combinations of substituents envisioned by this disclosure are preferably those that result in the formation of stable or chemically feasible compounds.

The term “stable compounds,” as used herein, refers to compounds which possess sufficient stability to allow for their manufacture and which maintain the integrity of the compounds for a sufficient period of time to be useful for the purposes detailed herein (e.g., formulation into therapeutic products, intermediates for use in production of therapeutic compounds, isolatable or storable intermediates, and/or treating a disease or condition responsive to therapeutic agents).

As used herein, the term “stereoisomer” refers to both enantiomers and diastereomers.

It will be appreciated that certain compounds of this invention may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e. one stereoisomer). As used in the chemical structures disclosed herein, a “wavy” bond ( [symbol] ) to a stereogenic atom indicates a chiral center of unknown absolute stereochemistry (i.e. one stereoisomer). As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. As used in the chemical structures disclosed herein, a

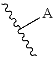

(i.e., a “wavy” line perpendicular to a “straight” bond to group “A”) indicates that group “A” is a substituent whose point of attachment is at the end of the bond that terminates at the “wavy” line.

Certain compounds disclosed herein may exist as tautomers and both tautomeric forms are intended, even though only a single tautomeric structure is depicted. For example, a description of Compound A is understood to include its tautomer Compound B and vice versa, as well as mixtures thereof.

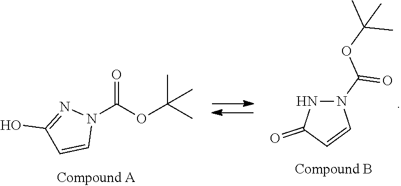

Unless otherwise stated, all tautomeric forms of the compounds of the disclosure are within the scope of the disclosure.

Unless otherwise stated, structures depicted herein are also meant to include all isomeric forms of the structure, e.g., geometric (or conformational), such as (Z) and (E) double bond isomers, and (Z) and (E) conformational isomers. Therefore, geometric and conformational mixtures of the compounds of the disclosure are within the scope of the disclosure.

As used herein, the term “pharmaceutically acceptable solid form” refers to a solid form of Compound I of this disclosure wherein the solid form (e.g., crystalline free form, crystalline salt, crystalline salt solvate, crystalline salt hydrate, and amorphous form) of Compound I is nontoxic and suitable for use in pharmaceutical compositions.

The terms “about” and “approximately,” when used in connection with doses, amounts, or weight percents of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. As used herein, the terms “about” and “approximately,” when used in connection with amounts, volumes, reaction times, reaction temperatures, etc., in methods or processes, may refer to an acceptable error for a particular value as determined by one of skill in the art, which depends in part on how the values is measured or determined. In some embodiments, the terms “about” and “approximately” mean within 1, 2, 3, or 4 standard deviations. In certain embodiments, the terms “about” and “approximately” mean within 30%, 25%, 20%, 15%, 10%, 9%, 8%, 7%, 6%, 5%, 4%, 3%, 2%, ₁%, 0.5%, 0.1%, or 0.05% of a given value or range. In some embodiments, the terms “about” and “approximately” mean within 15%, 10%, 5%, 4%, 3%, 2%, 1%, or 0.5% of a given value or range. In some embodiments, the terms “about” and “approximately” mean within 15% of a given value. In some embodiments, the terms “about” and “approximately” mean within 10% of a given value. As used herein, the symbol “-” appearing immediately before a numerical value has the same meaning as the terms “about” and “approximately.”

As used herein, the term “amorphous” refers to a solid material having no long-range order in the position of its molecules. Amorphous solids are generally glasses or supercooled liquids in which the molecules are arranged in a random manner so that there is no well-defined arrangement, e.g., molecular packing, and no long-range order. Amorphous solids are generally rather isotropic, i.e., exhibit similar properties in all directions and do not have definite melting points. Instead, they typically exhibit a glass transition temperature which marks a transition from glassy amorphous state to supercooled liquid amorphous state upon heating. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a representative comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, and solid state nuclear magnetic resonance (e.g., ¹³C, ¹⁹F, ¹⁵N, and ³¹P SSNMR). Accordingly, as used herein, the terms “crystalline Form A of Compound (I)” and “crystalline p-toluene sulfonic acid salt of Compound (I)” refer to unique crystalline forms that can be identified and distinguished from each other by one or more characterization techniques including, for example, XRPD, single crystal X-ray diffraction, and ¹³C SSNMR. In some embodiments, the novel crystalline forms are characterized by an X-ray powder diffractogram having one or more signals at one or more specified degree two-theta values (°2θ).

As used herein, the term “free form” refers to a non-ionized version of the compound in the solid state. Examples of free forms include free bases and free acids.

As used herein, the term “neat form” refers to an unsolvated and unhydrated free form version of a compound in the solid state.

As used herein, the term “solvate” refers to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate.”

In some embodiments, a solid material may comprise a mixture of crystalline solids and amorphous solids. A solid material comprising an amorphous compound may also, for example, contain up to 30% of a crystalline solid. In some embodiments, a solid material prepared to comprise an amorphous compound may also, for example, contain up to 25%, 20%, 15%, 10%, 5%, or 2% of a crystalline solid. In embodiments wherein the solid material contains a mixture of crystalline solids and amorphous solids, the characterizing data, such as XRPD, may contain indicators of both crystalline and amorphous solids. In some embodiments, a crystalline form of this disclosure may contain up to 30% amorphous compound. In some embodiments, a crystalline preparation of Compound I may contain up to 25%, 20%, 15%, 10%, 5%, or 2% of an amorphous solid.

As used herein, the term “substantially amorphous” refers to a solid material having little or no long-range order in the position of its molecules. For example, substantially amorphous materials have less than 15% crystallinity (e.g., less than 10% crystallinity, less than 5% crystallinity, or less than 2% crystallinity). It is also noted that the term “substantially amorphous” includes the descriptor, “amorphous,” which refers to materials having no (0%) crystallinity.

As used herein, the term “substantially crystalline” refers to a solid material having little or no amorphous molecules. For example, substantially crystalline materials have less than 15% amorphous molecules (e.g., less than 10% amorphous molecules, less than 5% amorphous molecules, or less than 2% amorphous molecules). It is also noted that the term “substantially crystalline” includes the descriptor “crystalline,” which refers to materials that are 100% crystalline form.

As used herein, the term “ambient conditions” means room temperature, open air condition and uncontrolled humidity condition. As used herein, the terms “room temperature” and “ambient temperature” mean 15° C. to 30° C.

As used herein, the terms “X-ray powder diffractogram,” “X-ray powder diffraction pattern,” “XRPD pattern,” “XRPD spectrum” interchangeably refer to an experimentally obtained pattern plotting signal positions (on the abscissa) versus signal intensities (on the ordinate).

A “signal” or “peak” as used herein refers to a point in the XRPD pattern where the intensity as measured in counts is at a local maximum. An XRPD peak is identified by its angular value as measured in degrees 2θ (° 2θ), depicted on the abscissa of an X-ray powder diffractogram, which may be expressed, for example, as “a signal at . . . degrees two-theta,” “a signal at [a] two-theta value(s) of . . . “and/or “a signal at at least . . . two-theta value(s) selected from . . . ”

The repeatability of the measured angular values is in the range of 0.2° 20, i.e., the angular value can be at the recited angular value+0.2 degrees two-theta, the angular value−0.2 degrees two-theta, or any value between those two end points (angular value+0.2 degrees two-theta and angular value −0.2 degrees two-theta).

One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular]Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta) generally mean that value is identified as +0.2 degrees two-theta of the reported value, an art-recognized variance.

As used herein, the term “TGA” refers to thermogravimetric analysis and “TGA/DSC” refers to thermogravimetric analysis and differential scnning calorimetry.

As used herein, the term “DSC” refers to the analytical method of differential scanning calorimetry.

As used herein, the term “glass transition temperature” or “Tg” refers to the temperature above which a hard and brittle “glassy” amorphous solid becomes viscous or rubbery.

As used herein, the term “melting temperature”, “melting point”, or “Tm” refers to the temperature at which a material transitions from a solid to a liquid phase.

As used herein, the term “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include, inter alia, a crystalline drug in an amorphous polymer; an amorphous drug in an amorphous polymer; an amorphous drug dispersed in an amorphous drug; or, alternatively, an amorphous drug dispersed in one or more excipients. In some embodiments, a solid dispersion includes the polymer constituting the dispersed phase, and the drug constitute the continuous phase. Or, a solid dispersion includes the drug constituting the dispersed phase, and the polymer constituting the continuous phase.

The disclosure also provides processes for preparing salts of the compounds of the disclosure.

A salt of a compound of this disclosure is formed between an acid and a basic group of the compound, such as an amino functional group, or a base and an acidic group of the compound, such as a carboxyl functional group. In some embodiments, the salt is a pharmaceutically acceptable salt.

As used herein, the term “pharmaceutically acceptable salt” means any non-toxic salt that, upon administration to a recipient, is capable of providing, either directly or indirectly, a compound of this disclosure. Pharmaceutically acceptable salts of the compounds of this disclosure include those derived from suitable inorganic and organic acids and bases. A “pharmaceutically acceptable counterion” is an ionic portion of a salt that is not toxic when released from the salt upon administration to a recipient. One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound.

A “free base” form of a compound does not contain an ionically bonded salt. It is noted that the disclosed amounts of the compounds or their pharmaceutically acceptable salts thereof herein are based upon their free base form. For example, “10 mg of at least one compound chosen from Compound I and pharmaceutically acceptable salts thereof” includes 10 mg of Compound I and a concentration of a pharmaceutically acceptable salt of Compound I equivalent to 10 mg of Compound I.

Suitable pharmaceutically acceptable salts are, for example, those disclosed in S. M. Berge, et al. J. Pharm. Sci., 1977, 66, 1-19. For example, Table 1 of that article provides the following pharmaceutically acceptable salts:

Table of Pharmaceutically Acceptable Salts

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | Triethiodide |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |

Non-limiting examples of pharmaceutically acceptable salts derived from appropriate acids include: salts formed with inorganic acids, such as hydrochloric acid, hydrobromic acid, phosphoric acid, sulfuric acid, and perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions, such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

In some embodiments, the disclosure also is directed to processes for preparing isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope-labelled). Examples of isotopes which are commercially available and suitable for the disclosure include isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²P, ³⁵S, ¹⁸F, and ³⁶Cl, respectively.

In the compounds of this disclosure, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition.

As used herein, the term “derivative” refers to a collection of molecules having a chemical structure identical to a compound of this disclosure, except that one or more atoms of the molecule may have been substituted with another atom. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure. Such compounds are useful as, for example, analytical tools, probes in biological assays, or compounds with improved therapeutic profiles.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group. In chemical structures, deuterium may be represented as “D”.

As used herein, the phrase “deuterated derivatives of [a compound]and its stereoisomers, and pharmaceutically acceptable salts of any of the foregoing” is intended to include deuterated derivatives of the specified compound, deuterated derivatives of any stereoisomers of that compound, and pharmaceutically acceptable salts of the specified compound, pharmaceutically acceptable salts of any of the stereoisomers of that compound, as well as pharmaceutically acceptable salts of deuterated derivatives of the specified compound or its stereoisomers.

In some embodiments, the derivative is a silicon derivative, in which at least one carbon atom in a disclosed compound has been replaced with silicon. In some embodiments, the at least one carbon atom replaced with silicon may be a non-aromatic carbon. In some embodiments, the at least one carbon atom replaced with silicon may be an aromatic carbon. In certain embodiments, the silicon derivatives of the invention may also have one or more hydrogen atoms replaced with deuterium and/or germanium.

In other embodiments, the derivative is a germanium derivative, in which at least one carbon atom in a disclosed compound has been replaced with germanium. In certain embodiments, the germanium derivatives of the invention may also have one or more hydrogen atoms replaced with deuterium and/or silicon.

Because the general properties of silicon and germanium are similar to those of carbon, replacement of carbon by silicon or germanium can result in compounds with similar biological activity to a carbon-containing original compound.

### Detailed Description of Embodiments

### Solid Forms

Another aspect of the disclosure provides solid forms of Compound I (e.g., crystalline forms, amorphous forms, solvates), which can be used in the methods of treatment and pharmaceutical compositions described herein. In some embodiments, the invention provides neat amorphous forms of Compound I. In some embodiments, the invention provides neat crystalline forms of Compound I. In some embodiments, the invention provides solvate crystalline forms of Compound I.

### A. Compound I Methanol Solvate (Wet)

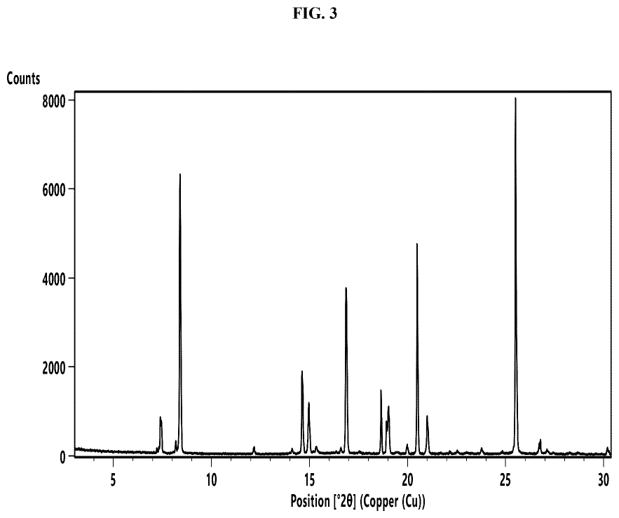

In some embodiments, the invention provides crystalline Compound I methanol solvate (wet). FIG. 3 provides an X-ray powder diffractogram of crystalline Compound I methanol solvate (wet).

In some embodiments, crystalline Compound I methanol solvate (wet) is substantially pure. In some embodiments, crystalline Compound I methanol solvate (wet) is substantially crystalline. In some embodiments, crystalline Compound I methanol solvate (wet) is characterized by an X-ray powder diffractogram generated by an X-ray powder diffraction analysis with an incident beam of Cu Kα radiation.

In some embodiments, crystalline Compound I methanol solvate (wet) is characterized by an X-ray powder diffractogram having a signal at 25.5±0.2 degrees two-theta. In some embodiments, crystalline Compound I methanol solvate (wet) is characterized by an X-ray powder diffractogram having a signal at 21.0±0.2 degrees two-theta. In some embodiments, crystalline Compound I methanol solvate (wet) is characterized by an X-ray powder diffractogram having a signal at 20.5±0.2 degrees two-theta. In some embodiments, crystalline Compound I methanol solvate (wet) is characterized by an X-ray powder diffractogram having a signal at 19.0±0.2 degrees two-theta. In some embodiments, crystalline Compound I methanol solvate (wet) is characterized by an X-ray powder diffractogram having a signal at 18.9±0.2 degrees two-theta. In some embodiments, crystalline Compound I methanol solvate (wet) is characterized by an X-ray powder diffractogram having a signal at 18.6±0.2 degrees two-theta. In some embodiments, crystalline Compound I methanol solvate (wet) is characterized by an X-ray powder diffractogram having a signal at 16.9±0.2 degrees two-theta. In some embodiments, crystalline Compound I methanol solvate (wet) is characterized by an X-ray powder diffractogram having a signal at 15.0±0.2 degrees two-theta. In some embodiments, crystalline Compound I methanol solvate (wet) is characterized by an X-ray powder diffractogram having a signal at 14.6±0.2 degrees two-theta. In some embodiments, crystalline Compound I methanol solvate (wet) is characterized by an X-ray powder diffractogram having a signal at 8.4±0.2 degrees two-theta.

In some embodiments, crystalline Compound I methanol solvate (wet) is characterized by an X-ray powder diffractogram having signals at two or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta. In some embodiments, crystalline Compound I methanol solvate (wet) is characterized by an X-ray powder diffractogram having signals at three or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta. In some embodiments, crystalline Compound I methanol solvate (wet) is characterized by an X-ray powder diffractogram having signals at four or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta. In some embodiments, crystalline Compound I methanol solvate (wet) is characterized by an X-ray powder diffractogram having signals at five or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta. In some embodiments, crystalline Compound I methanol solvate (wet) is characterized by an X-ray powder diffractogram having signals at six or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

In some embodiments, crystalline Compound I methanol solvate (wet) is characterized by an X-ray powder diffractogram having signals at 25.5±0.2 degrees two-theta and 8.4±0.2 degrees two-theta. In some embodiments, crystalline Compound I methanol solvate (wet) is characterized by an X-ray powder diffractogram having signals at 20.5±0.2 degrees two-theta and 8.4±0.2 degrees two-theta. In some embodiments, crystalline Compound I methanol solvate (wet) is characterized by an X-ray powder diffractogram having signals at 20.5±0.2 degrees two-theta and 16.9±0.2 degrees two-theta. In some embodiments, crystalline Compound I methanol solvate (wet) is characterized by an X-ray powder diffractogram having signals at 25.5±0.2 degrees two-theta, 20.5±0.2 degrees two-theta, and 8.4±0.2 degrees two-theta. In some embodiments, crystalline Compound I methanol solvate (wet) is characterized by an X-ray powder diffractogram having signals at 20.5±0.2 degrees two-theta, 16.9±0.2 degrees two-theta, and 8.4±0.2 degrees two-theta. In some embodiments, crystalline Compound I methanol solvate (wet) is characterized by an X-ray powder diffractogram having signals at 25.5±0.2 degrees two-theta, 20.5±0.2 degrees two-theta, 16.9±0.2 degrees two-theta, and 8.4±0.2 degrees two-theta.

In some embodiments, crystalline Compound I methanol solvate (wet) is characterized by an X-ray powder diffractogram having signals at 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

In some embodiments, crystalline Compound I methanol solvate (wet) is characterized by an X-ray powder diffractogram substantially similar to FIG. 3.

In some embodiments, methanol solvate (wet) is characterized by a monoclinic crystal system, P2₁ space group, and the following unit cell dimensions measured at 100 K on a Rigaku diffractometer equipped with Cu Kₐ radiation (λ=1.54178 Å):

| | | | |
|---|---|---|---|
| a | 6.7 ± 0.1 Å | α | 90° |
| b | 41.5 ± 0.1 Å | β | 92.1 ± 0.1° |
| c | 14.2 ± 0.1 Å | γ | 90°. |

In some embodiments, crystalline Compound methanol solvate (wet) is characterized as having a ¹³C SSNMR spectrum with a peak at 164.2±0.2 ppm. In some embodiments, crystalline Compound methanol solvate (wet) is characterized as having a ¹³C SSNMR spectrum with a peak at 162.8±0.2 ppm. In some embodiments, crystalline Compound I methanol solvate (wet) is characterized as having a ¹³C SSNMR spectrum with a signal at 145.6±0.2 ppm. In some embodiments, crystalline Compound methanol solvate (wet) is characterized as having a ¹³C SSNMR spectrum with a peak at 132.5±0.2 ppm. In some embodiments, crystalline Compound methanol solvate (wet) is characterized as having a ¹³C SSNMR spectrum with a peak at 124.5±0.2 ppm. In some embodiments, crystalline Compound methanol solvate (wet) is characterized as having a ¹³C SSNMR spectrum with a peak at 122.1±0.2 ppm. In some embodiments, crystalline Compound methanol solvate (wet) is characterized as having a ¹³C SSNMR spectrum with a peak at 120.6±0.2 ppm. In some embodiments, crystalline Compound I methanol solvate (wet) is characterized as having a ¹³C SSNMR spectrum with a signal at 113.1±0.2 ppm. In some embodiments, crystalline Compound I methanol solvate (wet) is characterized as having a ¹³C SSNMR spectrum with a signal at 73.5±0.2 ppm. In some embodiments, crystalline Compound I methanol solvate (wet) is characterized as having a ¹³C SSNMR spectrum with a signal at 55.9±0.2 ppm. In some embodiments, crystalline Compound methanol solvate (wet) is characterized as having a ¹³C SSNMR spectrum with a peak at 49.7±0.2 ppm. In some embodiments, crystalline Compound I methanol solvate (wet) is characterized as having a ¹³C SSNMR spectrum with a signal at 35.2±0.2 ppm. In some embodiments, crystalline Compound I methanol solvate (wet) is characterized as having a ¹³C SSNMR spectrum with a signal at 31.1±0.2 ppm. In some embodiments, crystalline Compound I methanol solvate (wet) is characterized as having a ¹³C SSNMR spectrum with a signal at 30.5±0.2 ppm. In some embodiments, crystalline Compound methanol solvate (wet) is characterized as having a ¹³C SSNMR spectrum with a peak at 29.3±0.2 ppm. In some embodiments, crystalline Compound methanol solvate (wet) is characterized as having a ¹³C SSNMR spectrum with a peak at 27.4±0.2 ppm. In some embodiments, crystalline Compound I methanol solvate (wet) is characterized as having a ¹³C SSNMR spectrum with a signal at 24.8±0.2 ppm. In some embodiments, crystalline Compound methanol solvate (wet) is characterized as having a ¹³C SSNMR spectrum with a peak at 21.0±0.2 ppm. In some embodiments, crystalline Compound I methanol solvate (wet) is characterized as having a ¹³C SSNMR spectrum with a signal at 19.0±0.2 ppm.

In some embodiments, crystalline Compound I methanol solvate (wet) is characterized as having a ¹³C SSNMR spectrum with one or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with two or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with three or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with four or more signals 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with five or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with six or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with seven or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm. In some embodiments, crystalline Compound I methanol solvate (wet) is characterized as having a ¹³C SSNMR spectrum with eight or more signals selected from 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm.

In some embodiments, crystalline Compound I methanol solvate (wet) is characterized as having a ¹³C SSNMR spectrum with signals at 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, and 113.1±0.2 ppm. In some embodiments, crystalline Compound I methanol solvate (wet) is characterized as having a ¹³C SSNMR spectrum with signals at 164.2±0.2 ppm, 162.8±0.2 ppm, 145.6±0.2 ppm, 132.5±0.2 ppm, 124.5±0.2 ppm, 122.1±0.2 ppm, 120.6±0.2 ppm, 113.1±0.2 ppm, 73.5±0.2 ppm, 55.9±0.2 ppm, 49.7±0.2 ppm, 35.2±0.2 ppm, 31.1±0.2 ppm, 30.5±0.2 ppm, 29.3±0.2 ppm, 27.4±0.2 ppm, 24.8±0.2 ppm, 21.0±0.2 ppm, and 19.0±0.2 ppm.

In some embodiments, crystalline Compound I methanol solvate (wet) is characterized as having a ¹³C SSNMR spectrum with two or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with three or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with four or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with five or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with six or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with seven or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with eight or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm. In some embodiments, crystalline Compound I methanol solvate (wet) is characterized as having a ¹³C SSNMR spectrum with nine or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

In some embodiments, crystalline Compound I methanol solvate (wet) is characterized as having a ¹³C SSNMR spectrum with signals at 145.6±0.2 ppm and 132.5±0.2 ppm. In some embodiments, crystalline Compound I methanol solvate (wet) is characterized as having a ¹³C SSNMR spectrum with signals at 145.6±0.2 ppm, 132.5±0.2 ppm, and 113.1±0.2 ppm. In some embodiments, crystalline Compound I methanol solvate (wet) is characterized as having a ¹³C SSNMR spectrum with signals at 145.6±0.2 ppm, 132.5±0.2 ppm, 113.1±0.2 ppm, and 73.5±0.2 ppm. In some embodiments, crystalline Compound I methanol solvate (wet) is characterized as having a ¹³C SSNMR spectrum with signals at 145.6±0.2 ppm, 132.5±0.2 ppm, 113.1±0.2 ppm, 73.5±0.2 ppm, and 55.9±0.2 ppm. In some embodiments, crystalline Compound I methanol solvate (wet) is characterized as having a ¹³C SSNMR spectrum with signals at 145.6±0.2 ppm, 132.5±0.2 ppm, 113.1±0.2 ppm, 73.5±0.2 ppm, 55.9±0.2 ppm, and 35.2±0.2 ppm.

In some embodiments, crystalline Compound I methanol solvate (wet) is characterized as having a ¹³C SSNMR spectrum with signals at 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

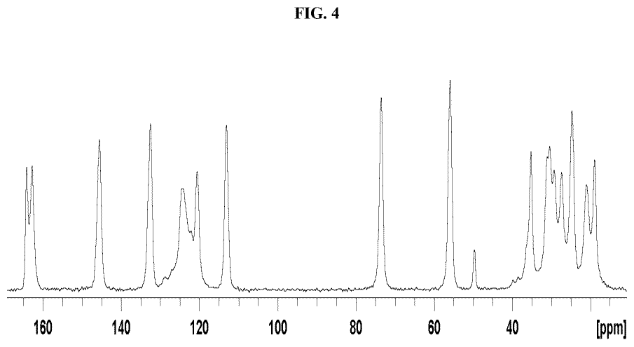

In some embodiments, crystalline Compound I methanol solvate (wet) is characterized by a ¹³C SSNMR spectrum substantially similar to FIG. 4.

In some embodiments, crystalline Compound I methanol solvate (wet) is characterized as having a ¹⁹F MAS signal at −63.9±0.2 ppm. In some embodiments, crystalline Compound I methanol solvate (wet) is characterized as having a ¹⁹F MAS signal at −76.6±0.2 ppm. In some embodiments, crystalline Compound I methanol solvate (wet) is characterized as having a ¹⁹F MAS signal at −79.7±0.2 ppm.

In some embodiments, crystalline Compound I methanol solvate (wet) is characterized as having a ¹⁹F MAS with two signals selected from −63.9±0.2 ppm, −76.6±0.2 ppm, and −79.7±0.2 ppm. In some embodiments, crystalline Compound I methanol solvate (wet) is characterized as having ¹⁹F MAS signals at −63.9±0.2 ppm, −76.6±0.2 ppm, and −79.7±0.2 ppm.

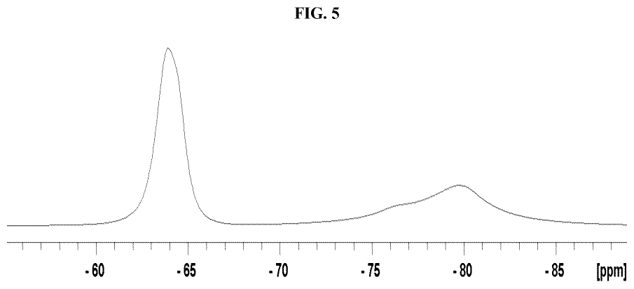

In some embodiments, crystalline Compound I methanol solvate (wet) is characterized by a ¹⁹F MAS substantially similar to FIG. 5.

Another aspect of the invention provides a method of making crystalline Compound I methanol solvate (wet). In some embodiments, the method of making crystalline Compound I methanol solvate (wet) comprises: (i) combining Compound I neat Form A, water and methanol in a sealed vial, (ii) heating to 65° C. and stirring until a homogeneous slurry is formed, and (iii) cooling the slurry without stirring and allowing to sit at room temperature over 3 days, to yield crystalline Compound I methanol solvate (wet).

B. Compound I Methanol Solvate (dry)

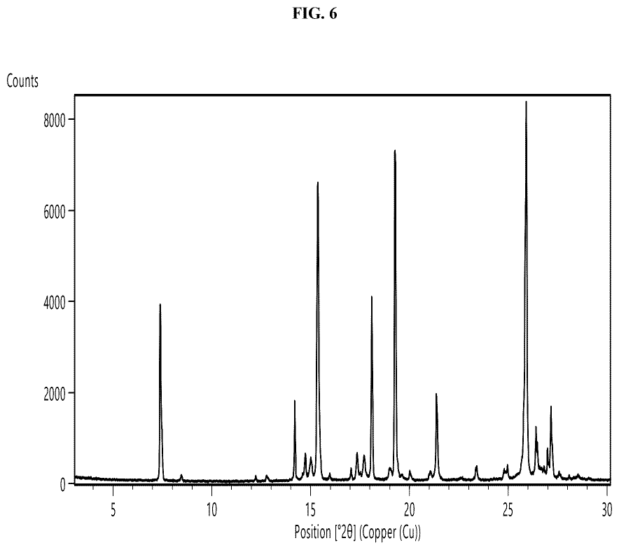

In some embodiments, the invention provides crystalline Compound I methanol solvate (dry). FIG. 6 provides an X-ray powder diffractogram of crystalline Compound I methanol solvate (dry).

In some embodiments, crystalline Compound I methanol solvate (dry) is substantially pure. In some embodiments, crystalline Compound I methanol solvate (dry) is substantially crystalline. In some embodiments, crystalline Compound I methanol solvate (dry) is characterized by an X-ray powder diffractogram generated by an X-ray powder diffraction analysis with an incident beam of Cu Kα radiation.

In some embodiments, crystalline Compound I methanol solvate (dry) is characterized by an X-ray powder diffractogram having a signal at 27.2±0.2 degrees two-theta. In some embodiments, crystalline Compound I methanol solvate (dry) is characterized by an X-ray powder diffractogram having a signal at 26.4±0.2 degrees two-theta. In some embodiments, crystalline Compound I methanol solvate (dry) is characterized by an X-ray powder diffractogram having a signal at 25.9±0.2 degrees two-theta. In some embodiments, crystalline Compound I methanol solvate (dry) is characterized by an X-ray powder diffractogram having a signal at 21.4±0.2 degrees two-theta. In some embodiments, crystalline Compound I methanol solvate (dry) is characterized by an X-ray powder diffractogram having a signal at 19.3±0.2 degrees two-theta. In some embodiments, crystalline Compound I methanol solvate (dry) is characterized by an X-ray powder diffractogram having a signal at 18.1±0.2 degrees two-theta. In some embodiments, crystalline Compound I methanol solvate (dry) is characterized by an X-ray powder diffractogram having a signal at 15.4±0.2 degrees two-theta. In some embodiments, crystalline Compound I methanol solvate (dry) is characterized by an X-ray powder diffractogram having a signal at 14.2±0.2 degrees two-theta. In some embodiments, crystalline Compound I methanol solvate (dry) is characterized by an X-ray powder diffractogram having a signal at 7.4±0.2 degrees two-theta.

In some embodiments, crystalline Compound I methanol solvate (dry) is characterized by an X-ray powder diffractogram having signals at two or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta. In some embodiments, crystalline Compound I methanol solvate (dry) is characterized by an X-ray powder diffractogram having signals at three or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta. In some embodiments, crystalline Compound I methanol solvate (dry) is characterized by an X-ray powder diffractogram having signals at four or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta. In some embodiments, crystalline Compound I methanol solvate (dry) is characterized by an X-ray powder diffractogram having signals at five or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta. In some embodiments, crystalline Compound I methanol solvate (dry) is characterized by an X-ray powder diffractogram having signals at six or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

In some embodiments, crystalline Compound I methanol solvate (dry) is characterized by an X-ray powder diffractogram having signals at 25.9±0.2 degrees two-theta and 19.3±0.2 degrees two-theta. In some embodiments, crystalline Compound I methanol solvate (dry) is characterized by an X-ray powder diffractogram having signals at 19.3±0.2 degrees two-theta and 15.4±0.2 degrees two-theta. In some embodiments, crystalline Compound I methanol solvate (dry) is characterized by an X-ray powder diffractogram having signals at 25.9±0.2 degrees two-theta, 19.3±0.2 degrees two-theta, and 15.4±0.2 degrees two-theta. In some embodiments, crystalline Compound I methanol solvate (dry) is characterized by an X-ray powder diffractogram having signals at 19.3±0.2 degrees two-theta, 18.1±0.2 degrees two-theta, and 15.4±0.2 degrees two-theta. In some embodiments, crystalline Compound I methanol solvate (dry) is characterized by an X-ray powder diffractogram having signals at 25.9±0.2 degrees two-theta, 19.3±0.2 degrees two-theta, 18.1±0.2 degrees two-theta, and 15.4±0.2 degrees two-theta.

In some embodiments, crystalline Compound I methanol solvate (dry) is characterized by an X-ray powder diffractogram having signals at 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

In some embodiments, crystalline Compound I methanol solvate (dry) is characterized by an X-ray powder diffractogram substantially similar to FIG. 6.

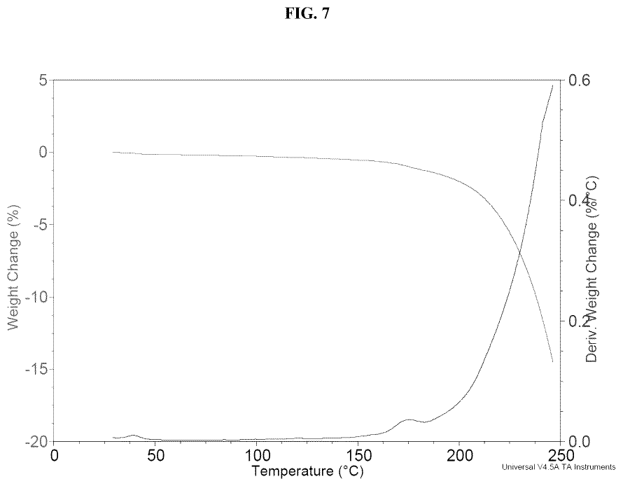

In some embodiments, Compound I methanol solvate (dry) is characterized by a TGA thermogram showing a loss of approximately 1.2% from ambient temperature up to about 182° C. In some embodiments, Compound I methanol solvate (dry) is characterized by a TGA thermogram substantially similar to FIG. 7.

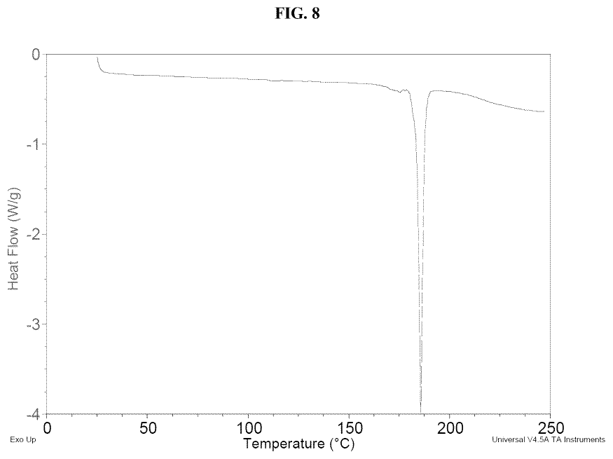

In some embodiments, Compound I methanol solvate (dry) is characterized by a DSC thermogram showing endotherms at about 175° C. and at about 186° C. In some embodiments, Compound I methanol solvate (dry) is characterized by a DSC thermogram substantially similar to FIG. 8.

Another aspect of the invention provides a method of making crystalline Compound I methanol solvate (dry). In some embodiments, the method of making crystalline Compound I methanol solvate (dry) comprises: (i) combining Compound I neat Form A, water and methanol in a sealed vial, (ii) heating to 65° C. and stirring until a homogeneous slurry is formed, (iii) cooling the slurry without stirring and allowing it to sit at room temperature over 3 days, and (iv) drying in a vacuum oven at 60° C. overnight to yield crystalline Compound I methanol solvate (dry).

C. Crystalline Compound I p-Toluene Sulfonic Acid

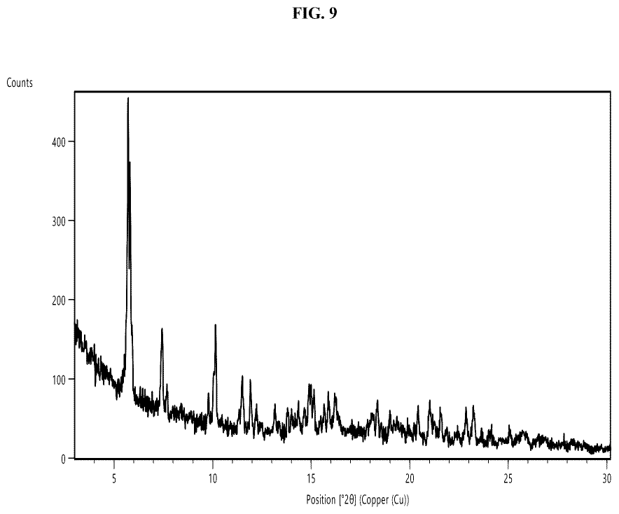

In some embodiments, the invention provides crystalline Compound I p-toluene sulfonic acid. FIG. 9 provides an X-ray powder diffractogram of crystalline Compound I p-toluene sulfonic acid.

In some embodiments, crystalline Compound I p-toluene sulfonic acid is substantially pure. In some embodiments, crystalline Compound I p-toluene sulfonic acid is substantially crystalline. In some embodiments, crystalline Compound I p-toluene sulfonic acid is characterized by an X-ray powder diffractogram generated by an X-ray powder diffraction analysis with an incident beam of Cu Kα radiation.

In some embodiments, crystalline Compound I p-toluene sulfonic acid is characterized by an X-ray powder diffractogram having a signal at 5.7±0.2 degrees two-theta. In some embodiments, crystalline Compound I p-toluene sulfonic acid is characterized by an X-ray powder diffractogram having a signal at 5.8±0.2 degrees two-theta. In some embodiments, crystalline Compound I p-toluene sulfonic acid is characterized by an X-ray powder diffractogram having a signal at 7.4±0.2 degrees two-theta. In some embodiments, crystalline Compound I p-toluene sulfonic acid is characterized by an X-ray powder diffractogram having a signal at 10.1±0.2 degrees two-theta. In some embodiments, crystalline Compound I p-toluene sulfonic acid is characterized by an X-ray powder diffractogram having a signal at 11.5±0.2 degrees two-theta. In some embodiments, crystalline Compound I p-toluene sulfonic acid is characterized by an X-ray powder diffractogram having a signal at 11.9±0.2 degrees two-theta. In some embodiments, crystalline Compound I p-toluene sulfonic acid is characterized by an X-ray powder diffractogram having a signal at 14.9±0.2 degrees two-theta. In some embodiments, crystalline Compound I p-toluene sulfonic acid is characterized by an X-ray powder diffractogram having a signal at 15.9±0.2 degrees two-theta. In some embodiments, crystalline Compound I p-toluene sulfonic acid is characterized by an X-ray powder diffractogram having a signal at 16.2±0.2 degrees two-theta. In some embodiments, crystalline Compound I p-toluene sulfonic acid is characterized by an X-ray powder diffractogram having a signal at 18.3±0.2 degrees two-theta. In some embodiments, crystalline Compound I p-toluene sulfonic acid is characterized by an X-ray powder diffractogram having a signal at 20.4±0.2 degrees two-theta. In some embodiments, crystalline Compound I p-toluene sulfonic acid is characterized by an X-ray powder diffractogram having a signal at 21.0±0.2 degrees two-theta. In some embodiments, crystalline Compound I p-toluene sulfonic acid is characterized by an X-ray powder diffractogram having a signal at 21.6±0.2 degrees two-theta. In some embodiments, crystalline Compound I p-toluene sulfonic acid C is characterized by an X-ray powder diffractogram having a signal at 22.8±0.2 degrees two-theta. In some embodiments, crystalline Compound I p-toluene sulfonic acid is characterized by an X-ray powder diffractogram having a signal at 23.2±0.2 degrees two-theta.

In some embodiments, crystalline Compound I p-toluene sulfonic acid is characterized by an X-ray powder diffractogram having signals at two or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta. In some embodiments, crystalline Compound I p-toluene sulfonic acid is characterized by an X-ray powder diffractogram having signals at three or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta. In some embodiments, crystalline Compound I p-toluene sulfonic acid is characterized by an X-ray powder diffractogram having signals at four or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta. In some embodiments, crystalline Compound I p-toluene sulfonic acid is characterized by an X-ray powder diffractogram having signals at five or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta. In some embodiments, crystalline Compound I p-toluene sulfonic acid is characterized by an X-ray powder diffractogram having signals at six or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

In some embodiments, crystalline Compound I p-toluene sulfonic acid is characterized by an X-ray powder diffractogram having signals at 5.7±0.2 degrees two-theta and 5.8±0.2 degrees two-theta. In some embodiments, crystalline Compound I p-toluene sulfonic acid is characterized by an X-ray powder diffractogram having signals at 5.7±0.2 degrees two-theta, 5.8±0.2 degrees two-theta, and 7.4±0.2 degrees two-theta. In some embodiments, crystalline Compound I p-toluene sulfonic acid is characterized by an X-ray powder diffractogram having signals at 5.7±0.2 degrees two-theta, 5.8±0.2 degrees two-theta, 7.4±0.2 degrees two-theta, and 10.1±0.2 degrees two-theta. In some embodiments, crystalline Compound I p-toluene sulfonic acid is characterized by an X-ray powder diffractogram having signals at 5.7±0.2 degrees two-theta, 5.8±0.2 degrees two-theta, 7.4±0.2 degrees two-theta, 10.1±0.2 degrees two-theta, and 11.5±0.2 degrees two-theta. In some embodiments, crystalline Compound I p-toluene sulfonic acid is characterized by an X-ray powder diffractogram having signals at 5.7±0.2 degrees two-theta, 5.8±0.2 degrees two-theta, 7.4±0.2 degrees two-theta, 10.1±0.2 degrees two-theta, 11.5±0.2 degrees two-theta, and 11.9±0.2 degrees two-theta.

In some embodiments, crystalline Compound I p-toluene sulfonic acid is characterized by an X-ray powder diffractogram substantially similar to FIG. 9.

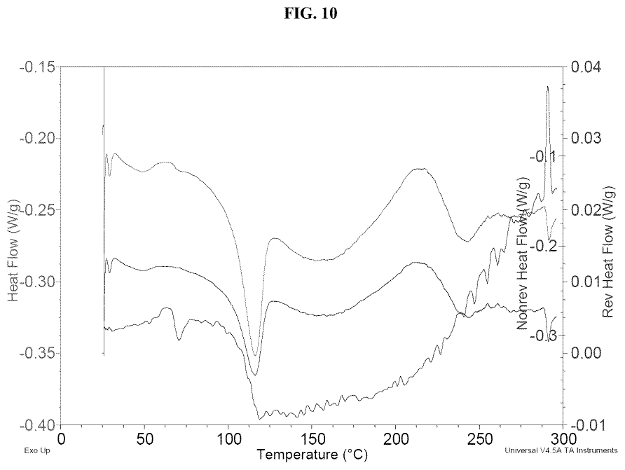

In some embodiments, Compound I p-toluene sulfonic acid is characterized by a DSC thermogram showing endotherms at about 48° C. and at about 115° C. In some embodiments, Compound I p-toluene sulfonic acid is characterized by a DSC thermogram substantially similar to FIG. 10.

In some embodiments, crystalline Compound I p-toluene sulfonic acid is characterized as having a ¹³C SSNMR spectrum with a signal at 163.8±0.2 ppm. In some embodiments, crystalline Compound I p-toluene sulfonic acid is characterized as having a ¹³C SSNMR spectrum with a signal at 161.8±0.2 ppm. In some embodiments, crystalline Compound I p-toluene sulfonic acid is characterized as having a ¹³C SSNMR spectrum with a signal at 160.9±0.2 ppm. In some embodiments, crystalline Compound I p-toluene sulfonic acid is characterized as having a ¹³C SSNMR spectrum with a signal at 152.9±0.2 ppm. In some embodiments, crystalline Compound I p-toluene sulfonic acid is characterized as having a ¹³C SSNMR spectrum with a signal at 146.1±0.2 ppm. In some embodiments, crystalline Compound I p-toluene sulfonic acid is characterized as having a ¹³C SSNMR spectrum with a signal at 141.0±0.2 ppm. In some embodiments, crystalline Compound I p-toluene sulfonic acid is characterized as having a ¹³C SSNMR spectrum with a signal at 139.3±0.2 ppm. In some embodiments, crystalline Compound I p-toluene sulfonic acid is characterized as having a ¹³C SSNMR spectrum with a signal at 138.0±0.2 ppm. In some embodiments, crystalline Compound I p-toluene sulfonic acid is characterized as having a ¹³C SSNMR spectrum with a signal at 136.7±0.2 ppm. In some embodiments, crystalline Compound I p-toluene sulfonic acid is characterized as having a ¹³C SSNMR spectrum with a signal at 132.5±0.2 ppm. In some embodiments, crystalline Compound I p-toluene sulfonic acid is characterized as having a ¹³C SSNMR spectrum with a signal at 130.6±0.2 ppm. In some embodiments, crystalline Compound I p-toluene sulfonic acid is characterized as having a ¹³C SSNMR spectrum with a signal at 127.8±0.2 ppm. In some embodiments, crystalline Compound I p-toluene sulfonic acid is characterized as having a ¹³C SSNMR spectrum with a signal at 126.7±0.2 ppm. In some embodiments, crystalline Compound I p-toluene sulfonic acid is characterized as having a ¹³C SSNMR spectrum with a signal at 124.7±0.2 ppm. In some embodiments, crystalline Compound I p-toluene sulfonic acid is characterized as having a ¹³C SSNMR spectrum with a signal at 122.1±0.2 ppm. In some embodiments, crystalline Compound I p-toluene sulfonic acid is characterized as having a ¹³C SSNMR spectrum with a signal at 114.5±0.2 ppm. In some embodiments, crystalline Compound I p-toluene sulfonic acid is characterized as having a ¹³C SSNMR spectrum with a signal at 113.1±0.2 ppm. In some embodiments, crystalline Compound I p-toluene sulfonic acid is characterized as having a ¹³C SSNMR spectrum with a signal at 110.3±0.2 ppm. In some embodiments, crystalline Compound I p-toluene sulfonic acid is characterized as having a ¹³C SSNMR spectrum with a signal at 75.5±0.2 ppm. In some embodiments, crystalline Compound I p-toluene sulfonic acid is characterized as having a ¹³C SSNMR spectrum with a signal at 57.0±0.2 ppm. In some embodiments, crystalline Compound I p-toluene sulfonic acid is characterized as having a ¹³C SSNMR spectrum with a signal at 48.6±0.2 ppm. In some embodiments, crystalline Compound I p-toluene sulfonic acid is characterized as having a ¹³C SSNMR spectrum with a signal at 35.0±0.2 ppm. In some embodiments, crystalline Compound I p-toluene sulfonic acid is characterized as having a ¹³C SSNMR spectrum with a signal at 32.5±0.2 ppm. In some embodiments, crystalline Compound I p-toluene sulfonic acid is characterized as having a ¹³C SSNMR spectrum with a signal at 31.0±0.2 ppm. In some embodiments, crystalline Compound I p-toluene sulfonic acid is characterized as having a ¹³C SSNMR spectrum with a signal at 29.8±0.2 ppm. In some embodiments, crystalline Compound I p-toluene sulfonic acid is characterized as having a ¹³C SSNMR spectrum with a signal at 28.3±0.2 ppm. In some embodiments, crystalline Compound I p-toluene sulfonic acid is characterized as having a ¹³C SSNMR spectrum with a signal at 26.2±0.2 ppm. In some embodiments, crystalline Compound I p-toluene sulfonic acid is characterized as having a ¹³C SSNMR spectrum with a signal at 25.0±0.2 ppm. In some embodiments, crystalline Compound I p-toluene sulfonic acid is characterized as having a ¹³C SSNMR spectrum with a signal at 23.0±0.2 ppm. In some embodiments, crystalline Compound I p-toluene sulfonic acid is characterized as having a ¹³C SSNMR spectrum with a signal at 19.5±0.2 ppm.

In some embodiments, crystalline Compound I p-toluene sulfonic acid is characterized as having a ¹³C SSNMR spectrum with two or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with three or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with four or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with five or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with six or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

In some embodiments, crystalline Compound I p-toluene sulfonic acid is characterized as having a ¹³C SSNMR spectrum with signals at 141.0±0.2 ppm and 19.5±0.2 ppm. In some embodiments, crystalline Compound I p-toluene sulfonic acid is characterized as having a ¹³C SSNMR spectrum with signals at 141.0±0.2 ppm, 23.0±0.2 ppm, and 19.5±0.2 ppm. In some embodiments, crystalline Compound I p-toluene sulfonic acid is characterized as having a ¹³C SSNMR spectrum with signals at 141.0±0.2 ppm, 57.0±0.2 ppm, 23.0±0.2 ppm, and 19.5±0.2 ppm. In some embodiments, crystalline Compound I p-toluene sulfonic acid is characterized as having a ¹³C SSNMR spectrum with signals at 141.0±0.2 ppm, 57.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with signals at 141.0±0.2 ppm, 57.0±0.2 ppm, 31.0±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with signals at 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

In some embodiments, crystalline Compound I p-toluene sulfonic acid is characterized as having a ¹³C SSNMR spectrum with one or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with two or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with three or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with four or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with five or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with six or more signals selected 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with seven or more signals selected 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm. In some embodiments, crystalline Compound I p-toluene sulfonic acid is characterized as having a ¹³C SSNMR spectrum with eight or more signals selected 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm.

In some embodiments, crystalline Compound I p-toluene sulfonic acid is characterized as having a ¹³C SSNMR spectrum with three or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, and 110.3±0.2 ppm. In some embodiments, crystalline Compound I p-toluene sulfonic acid is characterized as having a ¹³C SSNMR spectrum with four or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, and 110.3±0.2 ppm. In some embodiments, crystalline Compound I p-toluene sulfonic acid is characterized as having a ¹³C SSNMR spectrum with five or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, and 110.3±0.2 ppm. In some embodiments, crystalline Compound I p-toluene sulfonic acid is characterized as having a ¹³C SSNMR spectrum with six or more signals selected from 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, and 110.3±0.2 ppm.

In some embodiments, crystalline Compound I p-toluene sulfonic acid is characterized as having a ¹³C SSNMR spectrum with signals at 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, and 110.3±0.2 ppm. In some embodiments, crystalline Compound I p-toluene sulfonic acid is characterized as having a ¹³C SSNMR spectrum with signals at 163.8±0.2 ppm, 161.8±0.2 ppm, 160.9±0.2 ppm, 152.9±0.2 ppm, 146.1±0.2 ppm, 141.0±0.2 ppm, 139.3±0.2 ppm, 138.0±0.2 ppm, 136.7±0.2 ppm, 132.5±0.2 ppm, 130.6±0.2 ppm, 127.8±0.2 ppm, 126.7±0.2 ppm, 124.7±0.2 ppm, 122.1±0.2 ppm, 114.5±0.2 ppm, 113.1±0.2 ppm, 110.3±0.2 ppm, 75.5±0.2 ppm, 57.0±0.2 ppm, 48.6±0.2 ppm, 35.0±0.2 ppm, 32.5±0.2 ppm, 31.0±0.2 ppm, 29.8±0.2 ppm, 28.3±0.2 ppm, 26.2±0.2 ppm, 25.0±0.2 ppm, 23.0±0.2 ppm, and 19.5±0.2 ppm.

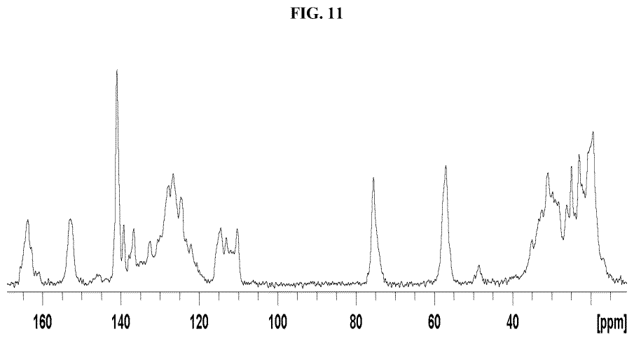

In some embodiments, crystalline Compound I p-toluene sulfonic acid is characterized by a ¹³C SSNMR spectrum substantially similar to FIG. 11.

In some embodiments, crystalline Compound I p-toluene sulfonic acid is characterized as having a ¹⁹F MAS signal at −62.5±0.2 ppm. In some embodiments, crystalline Compound I p-toluene sulfonic acid is characterized as having a ¹⁹F MAS signal at −64.2±0.2 ppm. In some embodiments, crystalline Compound I p-toluene sulfonic acid is characterized as having a ¹⁹F MAS signal at −64.6±0.2 ppm. In some embodiments, crystalline Compound I p-toluene sulfonic acid is characterized as having a ¹⁹F MAS signal at −65.4±0.2 ppm. In some embodiments, crystalline Compound I p-toluene sulfonic acid is characterized as having a ¹⁹F MAS signal at −66.1±0.2 ppm. In some embodiments, crystalline Compound I p-toluene sulfonic acid is characterized as having a ¹⁹F MAS signal at −77.4±0.2 ppm. In some embodiments, crystalline Compound I p-toluene sulfonic acid is characterized as having a ¹⁹F MAS signal at −78.1±0.2 ppm. In some embodiments, crystalline Compound I p-toluene sulfonic acid is characterized as having a ¹⁹F MAS signal at −79.7±0.2 ppm. In some embodiments, crystalline Compound I p-toluene sulfonic acid is characterized as having a ¹⁹F MAS signal at −80.1±0.2 ppm.

In some embodiments, crystalline Compound I p-toluene sulfonic acid is characterized as having a ¹⁹F MAS with two or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm. In some embodiments, crystalline Compound I p-toluene sulfonic acid is characterized as having a ¹⁹F MAS with three or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm. In some embodiments, crystalline Compound I p-toluene sulfonic acid is characterized as having a ¹⁹F MAS with four or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm. In some embodiments, crystalline Compound I p-toluene sulfonic acid is characterized as having a ¹⁹F MAS with five or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm. In some embodiments, crystalline Compound I p-toluene sulfonic acid is characterized as having a ¹⁹F MAS with six or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

In some embodiments, crystalline Compound I p-toluene sulfonic acid is characterized as having ¹⁹F MAS signals at −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

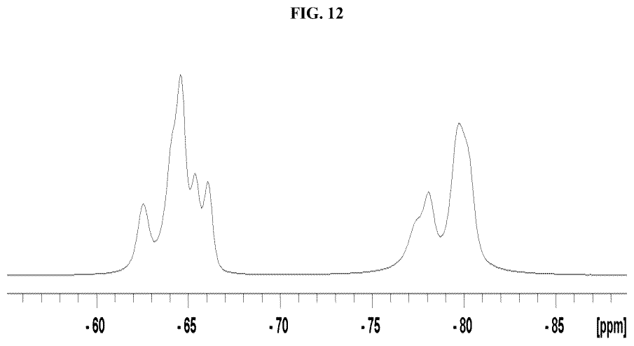

In some embodiments, crystalline Compound I p-toluene sulfonic acid is characterized by a ¹⁹F MAS substantially similar to FIG. 12.

Another aspect of the invention provides a method of making crystalline Compound I p-toluene sulfonic acid. In some embodiments, the method of making crystalline Compound I p-toluene sulfonic acid comprises: (i) adding p-toluene sulfonic acid to Compound I neat Form A in a milling ball tube, (ii) adding methanol and water (60:40 v/v), (iii) ball milling at 7500 rpm for 3 cycles of 60 seconds with 10 second pauses, and (iv) drying the resulting material in a vacuum dry oven at 40° C., to yield crystalline Compound I p-toluene sulfonic acid.

### D. Neat Amorphous Compound I

In some embodiments, the invention provides neat amorphous Compound I. In some embodiments, neat amorphous Compound I is substantially pure. In some embodiments, neat amorphous Compound I is substantially amorphous.

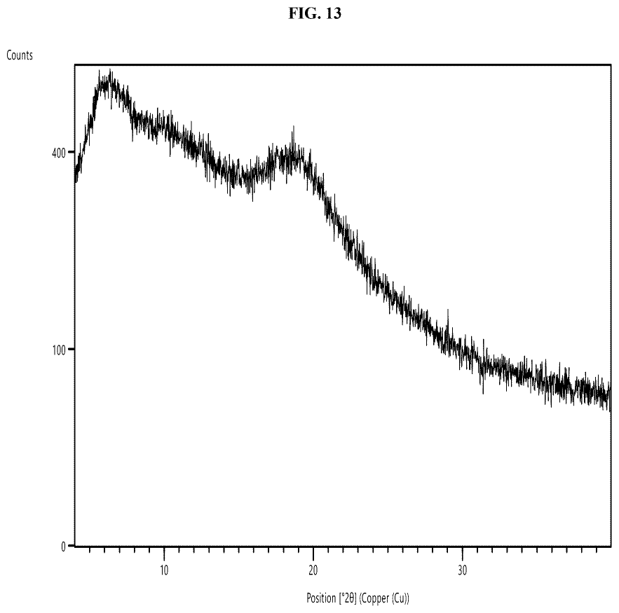

In some embodiments, neat amorphous Compound I is characterized by an X-ray powder diffractogram generated by an X-ray powder diffraction analysis with an incident beam of Cu Kα radiation. In some embodiments, neat amorphous Compound I is characterized by an X-ray powder diffractogram substantially similar to FIG. 13.

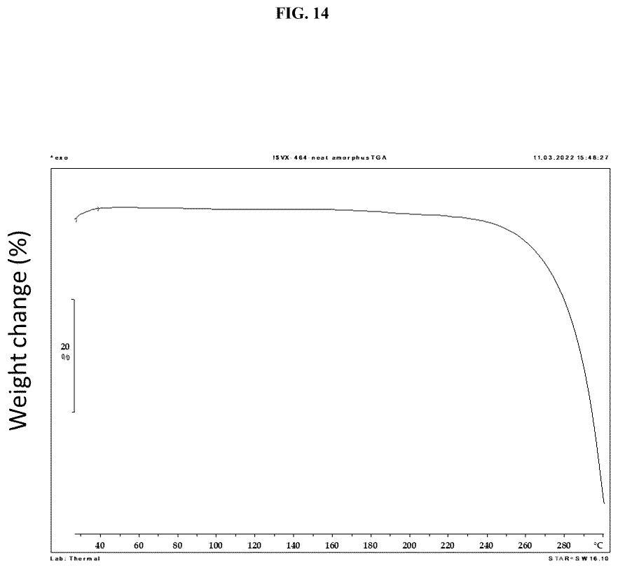

In some embodiments, a TGA thermogram of neat amorphous Compound I shows negligible weight loss from ambient temperature up until thermal degradation. In some embodiments, the TGA thermogram of neat amorphous Compound I is substantially similar to FIG. 14.

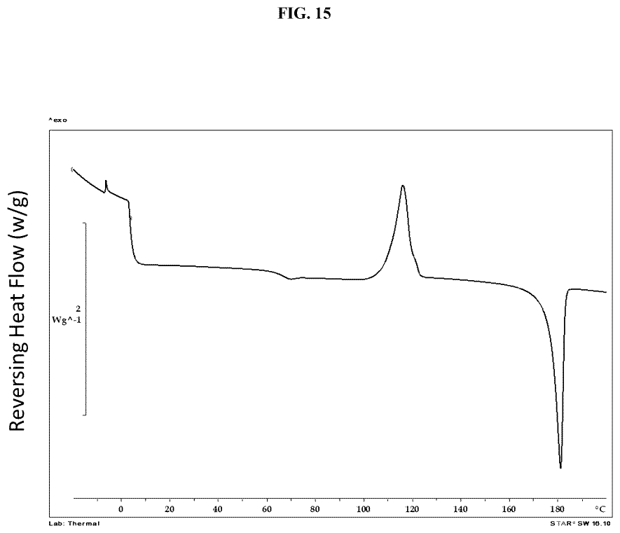

In some embodiments, the glass transition point of neat amorphous Compound I is measured using DSC. In some embodiments the glass transition of neat amorphous Compound I is 64.8° C. In some embodiments recrystallization of Compound I occurs at 110.2° C. and melting occurs at 181.1° C. In some embodiments, the DSC thermogram of neat amorphous Compound I is substantially similar to FIG. 15.

Another aspect of the invention provides a method of making neat amorphous Compound I. In some embodiments, the method of making neat amorphous Compound I comprises: (i) heating crystalline Compound I neat Form A to 200° C., and (ii) cooling the resulting material to 10° C. to yield neat amorphous Compound I.

Another aspect of the invention provides a method of making neat amorphous Compound I. In some embodiments, the method of making neat amorphous Compound I comprises: (i) heating crystalline Compound I neat Form A to 200° C. at a rate of 10° C. per minute, and (ii) cooling the resulting material to 10° C. to yield neat amorphous Compound I. Synthetic Processes and Intermediates

In some embodiments, Compound I is prepared using a compound of the disclosure.

In some embodiments, Compound I is prepared using a compound of Formula I, Formula II, or Formula III:

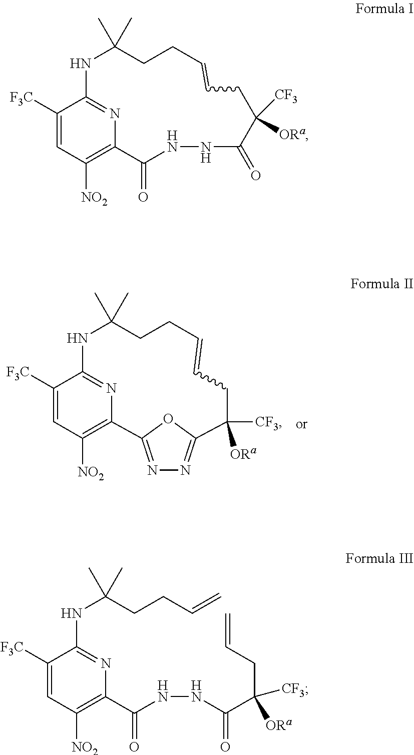

or a pharmaceutically acceptable salt thereof,
wherein:
R^{a} is selected from alcohol protecting groups; and
the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (EIZ mixture); and pharmaceutically acceptable salts thereof.

In some embodiments of Formula I, Formula II, and/or Formula III, R^{a} is selected from acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS). In some embodiments of Formula I, Formula II, and/or Formula III, R^{a} is Bn.

In some embodiments, Compound I is prepared using Compound 6:

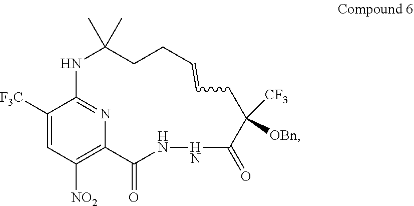 [SMILES: CC1(C)CCC=CC[C@](OCc2ccccc2)(C(F)(F)F)C(=O)NNC(=O)c2nc(c(C(F)(F)F)cc2[N+](=O)[O-])N1]

or a pharmaceutically acceptable salt thereof.

In some embodiments, Compound I is prepared using a compound selected from:

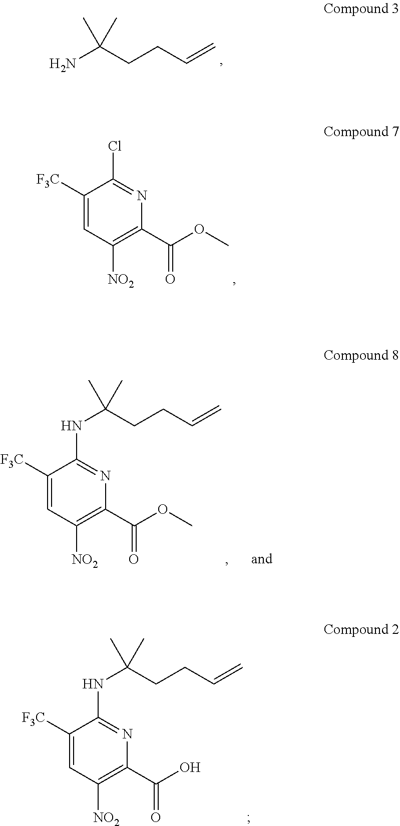

or a pharmaceutically acceptable salt thereof.

In some embodiments, Compound I is prepared using a compound selected from:

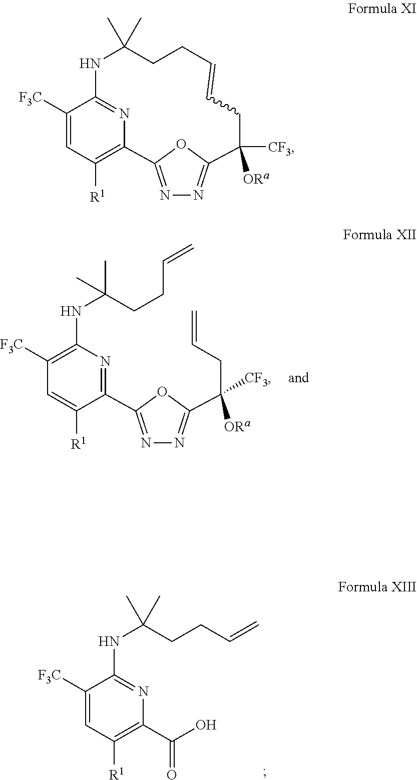

or a pharmaceutically acceptable salt thereof, wherein:
R^{a} is selected from alcohol protecting groups, and
R¹ is selected from —N(Boc)₂, −NHBoc, —N(Phth), —NH(Cbz), and —NO₂.

In some embodiments of Formula XI, Formula XII, and/or Formula XIII, R^{a} is selected from acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS). In some embodiments of Formula XI, Formula XII, and/or Formula XIII, R^{a} is Bn.

In some embodiments, Compound I is prepared using a compound selected from:

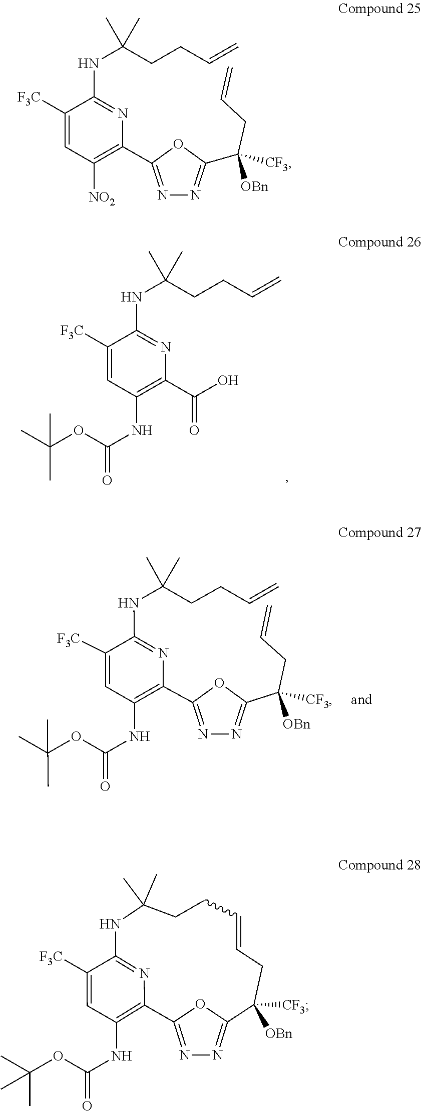

or a pharmaceutically acceptable salt thereof.

In some embodiments, a compound of the disclosure is a compound of Formula I, Formula II or Formula III:

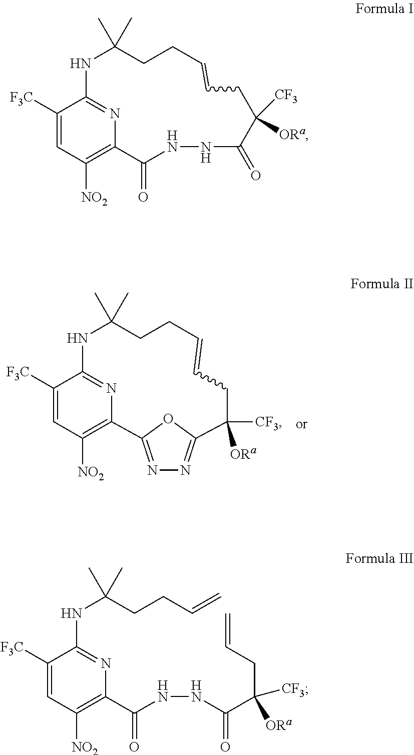

or a pharmaceutically acceptable salt thereof,
wherein:
R^{a} is selected from alcohol protecting groups; and
the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (EIZ mixture); and pharmaceutically acceptable salts thereof.

In some embodiments of Formula I, Formula II, and/or Formula III, R^{a} is selected from acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS). In some embodiments of Formula I, Formula II, and/or Formula III, R^{a} is Bn.

In some embodiments, a compound of the disclosure is a compound of Formula I:

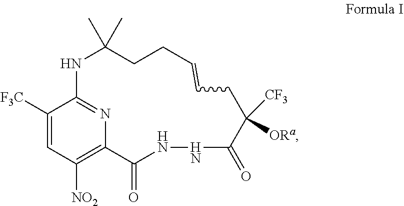

or a pharmaceutically acceptable salt thereof, wherein R^{a} is selected from alcohol protecting groups. In some embodiments of Formula I, R^{a} is selected from acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS). In some embodiments of Formula I, R^{a} is Bn.

In some embodiments, a compound of the disclosure is Compound 6:

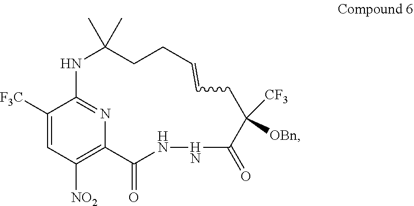 [SMILES: CC1(C)CCC=CC[C@](OCc2ccccc2)(C(F)(F)F)C(=O)NNC(=O)c2nc(c(C(F)(F)F)cc2[N+](=O)[O-])N1]

or a pharmaceutically acceptable salt thereof.

In some embodiments, a compound of the disclosure is

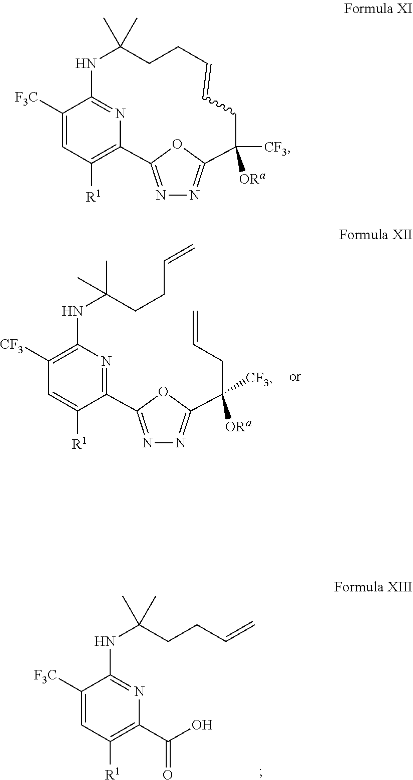

or a pharmaceutically acceptable salt thereof, wherein:
R^{a} is selected from alcohol protecting groups, and
R¹ is selected from —N(Boc)₂, —NHBoc, —N(Phth), —NH(Cbz), and —NO₂.

In some embodiments of Formula XI, Formula XII, and/or Formula XIII, R^{a} is selected from acetyl (Ac), benzoyl (Bz), benzyl (Bn), β-methoxyethoxymethyl (MEM), dimethoxytrityl (DMT), methoxymethyl (MOM), methoxytrityl (MMT), p-methoxybenzyl (PMB), pivaloyl (Piv), tetrahydropyranyl (THP), trityl (Tr), trimethylsilyl (TMS), triethylsilyl (TES), triisopropylsilyl (TIPS), t-butyldimethylsilyl (TBS), and t-butyldiphenylsilyl (TBDPS). In some embodiments of Formula XI, Formula XII, and/or Formula XIII, R^{a} is Bn.

In some embodiments, a compound of the disclosure is:

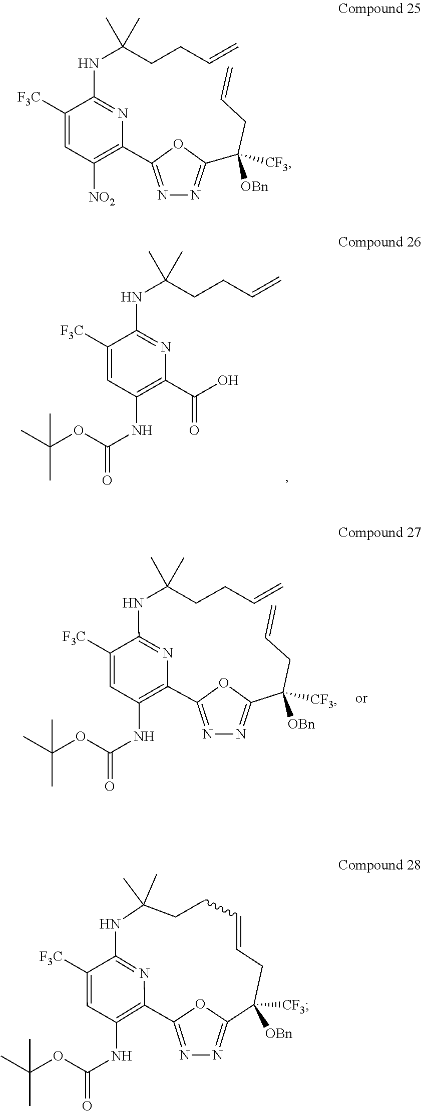

or a pharmaceutically acceptable salt thereof.

### Methods of Treatment

Compound I, in any one of the pharmaceutically acceptable solid forms disclosed herein, acts as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the invention provides methods of treating, lessening the severity of, or symptomatically treating cystic fibrosis in a patient comprising administering to the patient an effective amount of Compound I in any one of the pharmaceutically acceptable crystalline and amorphous forms disclosed herein, alone or in combination with another active ingredient, such as another CFTR modulating agent. In some embodiments, the patient has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments the patient is homozygous and has two F508del mutations. In some embodiments the patient is homozygous for the N1303K mutation.

In some embodiments, the patient is heterozygous and has an F508del mutation on one allele and a mutation on the other allele selected from the table below:

Table of CFTR Mutations
Mutation

| | | | | |
|---|---|---|---|---|
| Q2X | L218X | Q525X | R792X | E1104X |
| S4X | Q220X | G542X | E822X | W1145X |
| W19X | Y275X | G550X | W882X | R1158X |
| G27X | C276X | Q552X | W846X | R1162X |
| Q39X | Q290X | R553X | Y849X | S1196X |
| W57X | G330X | E585X | R851X | W1204X |
| E60X | W401X | G673X | Q890X | L1254X |
| R75X | Q414X | Q685X | S912X | S1255X |
| L88X | S434X | R709X | Y913X | W1282X |
| E92X | S466X | K710X | Q1042X | Q1313X |
| Q98X | S489X | Q715X | W1089X | Q1330X |
| Y122X | Q493X | L732X | Y1092X | E1371X |
| E193X | W496X | R764X | W1098X | Q1382X |
| W216X | C524X | R785X | R1102X | Q1411X |
| 185 + 1G→T | 711 + 5G→A | 1717 − 8G→A | 2622 + 1G→A | 3 + −1G→A |
| 296 + 1G→A | 712 − 1G→T | 1717 − 1G→A | 2790 − 1G→C | 3500 − 2A→G |
| 296 + 1G→T | 1248 + 1G→A | 1811 + 1G→C | 3040G→C | 3600 + 2insT |
| 405 + 1G→A | 1249 − 1G→A | 1811 + 1.6kbA→G | (G970R) | 3850 − 1G→A |
| 405 + 3A→C | 1341 + 1G→A | 1811 + 1643G→T | 3120G→A | 4005 + 1G→A |
| 406 − 1G→A | 1525 − 2A→G | 1812 − 1G→A | 3120 + 1G→A | 4374 + 1G→T |
| 621 + 1G→T | 1525 − 1G→A | 1898 + 1G→A | 3121 − 2A→G | |
| 711 + 1G→T | | 1898 + 1G→C | | |
| 182delT | 1078delT | 1677delTA | 2711delT | 3737delA |
| 306insA | 1119delA | 1782delA | 2732insA | 3791delC |
| 306delTAGA | 1138insG | 1824delA | 2869insG | 3821delT |
| 365 − 366insT | 1154insTC | 1833delT | 2896insAG | 3876delA |
| 394delTT | 1161delC | 2043delG | 2942insT | 3878delG |
| 442delA | 1213delT | 2143delT | 2957delT | 3905insT |
| 444delA | 1259insA | 2183AA→G | 3007delG | 4016insT |
| 457TAT→G | 1288insTA | 2184delA | 3028delA | 4021dupT |
| 541delC | 1343delG | 2184insA | 3171delC | 4022insT |
| 574delA | 1471delA | 2307insA | 3171insC | 4040delA |
| 663delT | 1497delGG | 2347delG | 3271delGG | 4279insA |
| 849delG | 1548delG | 2585delT | 3349insT | 4326delTC |
| 935delA | 1609del CA | 2594delGT | 3659delC | |
| CFTRdele1 | | CFTRdele16-17b | 1461ins4 | |
| CFTRdele2 | | CFTRdele 17a, 17b | 1924del7 | |
| CFTRdele2, 3 | | CFTRdele17a-18 | 2055del9→A | |
| CFTRdele2-4 | | CFTRdele19 | 2105-2117del13insAGAAA | |
| CFTRdele3-10, 14b-16 | | CFTRdele19-21 | 2372del8 | |
| CFTRdele4-7 | | CFTRdele21 | 2721del11 | |
| CFTRdele4-11 | | CFTRdele22-24 | 2991del32 | |
| CFTR50kbdel | | CFTRdele22, 23 | 3667ins4 | |
| CFTRdup6b-10 | | 124del23bp | 4010del4 | |
| CFTRdele11 | | 602del14 | 4209TGTT→AA | |
| CFTRdele13,14a | | 852del22 | | |
| CFTRdele14b-17b | | 991del5 | | |
| A46D | V520F | Y569D | N1303K | |
| G85E | A559T | L1065P | | |
| R347P | R560T | R1066C | | |
| L467P | R560S | L1077P | | |
| I507del | A561E | M1101K | | |

In some embodiments, the invention provides methods of treating, lessening the severity of, or symptomatically treating cystic fibrosis in a patient comprising administering to the patient an effective amount of Compound I in any one of the pharmaceutically acceptable solid (e.g., crystalline or amorphous) forms disclosed herein. In some embodiments, the pharmaceutically acceptable solid form of Compound I is a substantially amorphous form. In some embodiments, the pharmaceutically acceptable solid form of Compound I is a substantially crystalline form.

In some embodiments, the method of treating, lessening the severity of, or symptomatically treating cystic fibrosis in a patient comprises administering to the patient an effective amount of Compound I in any one of the pharmaceutically acceptable crystalline forms disclosed herein. In some embodiments, the pharmaceutically acceptable crystalline form of Compound I is Compound I methanol solvate (wet). In some embodiments, the pharmaceutically acceptable crystalline form of Compound I is Compound I methanol solvate (dry). In some embodiments, the pharmaceutically acceptable crystalline form of Compound I is Compound I p-toluene sulfonic acid.

In some embodiments, the method of treating, lessening the severity of, or symptomatically treating cystic fibrosis in a patient comprises administering to the patient an effective amount of Compound I in a pharmaceutically acceptable amorphous form disclosed herein. In some embodiments, the pharmaceutically acceptable form of Compound I is neat amorphous Compound I.

In some embodiments, the method of treating, lessening the severity of, or symptomatically treating cystic fibrosis in a patient comprises administering to the patient an effective amount of Compound I as any one of the pharmaceutically acceptable solid (e.g., crystalline or amorphous) forms disclosed herein in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator.

In some embodiments, the method of treating, lessening the severity of, or symptomatically treating cystic fibrosis in a patient comprises administering to the patient an effective amount of Compound I as any one of the pharmaceutically acceptable solid (e.g., crystalline or amorphous) forms disclosed herein in combination with at least one additional active pharmaceutical ingredient. In some embodiments, the at least one additional active pharmaceutical ingredient is selected from Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, the method of treating, lessening the severity of, or symptomatically treating cystic fibrosis in a patient comprises administering to the patient an effective amount of Compound I as a solid form selected from Compound I methanol solvate (wet), Compound I methanol solvate (dry), Compound I p-toluene sulfonic acid, and neat amorphouos Compound I, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, the method of treating, lessening the severity of, or symptomatically treating cystic fibrosis in a patient comprises administering to the patient an effective amount of Compound I as a solid crystalline form selected from Compound I methanol solvate (wet), Compound I methanol solvate (dry), Compound I p-toluene sulfonic acid, and neat amorphouos Compound I, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof

In some embodiments, the method of treating, lessening the severity of, or symptomatically treating cystic fibrosis in a patient comprises administering to the patient an effective amount of Compound I as a solid amorphous form that is neat amorphous Compound I, in combination with at least one additional active pharmaceutical ingredient. In some embodiments, the at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

### Pharmaceutical Compositions

Another aspect of the invention provides pharmaceutical compositions comprising Compound I in any one of the pharmaceutically acceptable solid (e.g., crystalline or amorphous) forms disclosed herein. In some embodiments, the pharmaceutical composition comprises Compound I in a solid form selected from Compound I methanol solvate (wet), Compound I methanol solvate (dry), and Compound I p-toluene sulfonic acid. In some embodiments, the pharmaceutical composition comprises neat amorphous Compound I.

In some embodiments, the invention provides pharmaceutical compositions comprising Compound I in any one of the pharmaceutically acceptable solid (e.g., crystalline or amorphous) forms disclosed herein in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises Compound I as any one of the pharmaceutically acceptable crystalline forms disclosed herein and at least two additional active pharmaceutical ingredients, one of which is a CFTR corrector and one of which is a CFTR potentiator. In some embodiments, at least one additional active pharmaceutical ingredient is Compound II, Compound III, Compound III-d, Compound IV, Compound V, Compound VI, Compound VII, Compound VIII, Compound IX, Compound X, and pharmaceutically acceptable salts and deuterated derivatives thereof.

In some embodiments, at least one additional active pharmaceutical ingredient is selected from mucolytic agents, bronchodilators, antibiotics, anti-infective agents, and anti-inflammatory agents.

In some embodiments, the invention provides a pharmaceutical composition comprising (a) Compound I in any one of the pharmaceutically acceptable solid (e.g., crystalline or amorphous) forms disclosed herein, and (b) at least one pharmaceutically acceptable carrier.

In some embodiments, the invention provides pharmaceutical compositions comprising (a) Compound I in any one of the pharmaceutically acceptable solid (e.g., crystalline or amorphous) forms disclosed herein, (b) at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof, and (c) at least one pharmaceutically acceptable carrier.

In some embodiments, the invention provides pharmaceutical compositions comprising (a) Compound I in a solid form selected from Compound I methanol solvate (wet), Compound I methanol solvate (dry), Compound I p-toluene sulfonic acid, and neat amorphouos Compound I, (b) at least one compound chosen from Compound II, Compound III, Compound III-d, Compound IV, Compound V, Compound VI, Compound VII, Compound VIII, Compound IX, Compound X, and pharmaceutically acceptable salts and deuterated derivatives thereof, and (c) at least one pharmaceutically acceptable carrier.

The pharmaceutical compositions described herein are useful for treating cystic fibrosis and other CFTR-mediated diseases.

As described above, pharmaceutical compositions disclosed herein may optionally further comprise at least one pharmaceutically acceptable carrier. The at least one pharmaceutically acceptable carrier may be selected from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D.B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclose various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Non-limiting Exemplary Embodiments

### A. Set 1

1. Compound I

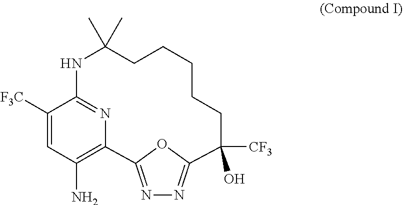 [SMILES: CC1(C)CCCCC[C@](O)(C(F)(F)F)C2=NN=C(O2)c2nc(c(C(F)(F)F)cc2N)N1]

as substantially amorphous Compound I neat amorphous form (i.e., wherein less than 15% of Compound I is in crystalline form, wherein less than 10% of Compound I is in crystalline form, wherein less than 5% of Compound I is in crystalline form).

2. The substantially amorphous Compound I neat amorphous form according to Embodiment 1, wherein Compound I is 100% amorphous.

3. The substantially amorphous Compound I neat amorphous form according to Embodiment 1 or Embodiment 2, characterized by an X-ray powder diffractogram substantially similar to FIG. 13.

4. The substantially amorphous Compound I neat amorphous form according to any one of Embodiments 1-3, characterized by a TGA thermogram showing negligible weight loss from ambient temperature up until thermal degradation.

5. The substantially amorphous Compound I neat amorphous form according to any one of Embodiments 1-4, characterized by a TGA thermogram substantially similar to FIG. 14.

6. The substantially amorphous Compound I neat amorphous form according to any one of Embodiments 1-5, characterized by a glass transition temperature of 64.8° C.

7. The substantially amorphous Compound I neat amorphous form according to any one of Embodiments 1-6, characterized by a DSC thermogram substantially similar to FIG. 15.

8. The substantially amorphous Compound I neat amorphous form according to any one of Embodiments 1-7, prepared by (i) heating crystalline Compound I Form A to 200° C., and (ii) cooling the resulting material to 10° C. to yield neat amorphous Compound I.

9. Substantially crystalline Compound I methanol solvate (wet) (i.e., wherein less than 15% of Compound I is in amorphous form, wherein less than 10% of Compound I is in amorphous form, wherein less than 5% of Compound I is in amorphous form).

10. The substantially crystalline Compound I methanol solvate (wet) according to Embodiment 9, wherein Compound I methanol solvate (wet) is 100% crystalline.

11. The substantially crystalline Compound I methanol solvate (wet) according to Embodiment 9 or Embodiment 10, characterized by an X-ray powder diffractogram having a signal at one or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

12. The substantially crystalline Compound I methanol solvate (wet) according to any one of Embodiments 9-11, characterized by an X-ray powder diffractogram having having a signal at two or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0 0.2 degrees two-theta, 14.6±0.2 degrees two-theta, and 8.4±0.2 degrees two-theta.

13. The substantially crystalline Compound I methanol solvate (wet) according to any one of Embodiments 9-12, characterized by an X-ray powder diffractogram having having a signal at three or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

14. The substantially crystalline Compound I methanol solvate (wet) according to any one of Embodiments 9-13, characterized by an X-ray powder diffractogram having having a signal at four or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

15. The substantially crystalline Compound I methanol solvate (wet) according to any one of Embodiments 9-14, characterized by an X-ray powder diffractogram having having a signal at five or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

16. The substantially crystalline Compound I methanol solvate (wet) according to any one of Embodiments 9-15, characterized by an X-ray powder diffractogram having having a signal at six or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

17. The substantially crystalline Compound I methanol solvate (wet) according to any one of Embodiments 9-16, characterized by an X-ray powder diffractogram having having signals at 25.5±0.2 degrees two-theta and 8.4±0.2 degrees two-theta.

18. The substantially crystalline Compound I methanol solvate (wet) according to any one of Embodiments 9-16, characterized by an X-ray powder diffractogram having having signals at 20.5±0.2 degrees two-theta and 8.4±0.2 degrees two-theta.

19. The substantially crystalline Compound I methanol solvate (wet) according to any one of Embodiments 9-16, characterized by an X-ray powder diffractogram having having signals at 20.5±0.2 degrees two-theta and 16.9±0.2 degrees two-theta.

20. The substantially crystalline Compound I methanol solvate (wet) according to any one of Embodiments 9-16, characterized by an X-ray powder diffractogram having having signals at 25.5±0.2 degrees two-theta, 20.5±0.2 degrees two-theta, and 8.4±0.2 degrees two-theta.

21. The substantially crystalline Compound I methanol solvate (wet) according to any one of Embodiments 9-16, characterized by an X-ray powder diffractogram having having signals at 20.5±0.2 degrees two-theta, 16.9±0.2 degrees two-theta, and 8.4±0.2 degrees two-theta.

22. The substantially crystalline Compound I methanol solvate (wet) according to any one of Embodiments 9-16, characterized by an X-ray powder diffractogram having having signals at 25.5±0.2 degrees two-theta, 20.5±0.2 degrees two-theta, 16.9±0.2 degrees two-theta, and 8.4±0.2 degrees two-theta.

23. The substantially crystalline Compound I methanol solvate (wet) according to any one of Embodiments 9-16, characterized by an X-ray powder diffractogram having having signals at 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

24. The substantially crystalline Compound I methanol solvate (wet) according to any one of Embodiments 9-23, characterized by an X-ray powder diffractogram substantially similar to FIG. 3.

25. The substantially crystalline Compound I methanol solvate (wet) according to any one of Embodiments 9-24, characterized by a monoclinic crystal system, P2₁ space group, and the following unit cell dimensions measured at 100 K on a Rigaku diffractometer equipped with Cu K_{α} radiation (λ=1.54178 Å):

| | | | |
|---|---|---|---|
| a | 6.7 ± 0.1 Å | α | 90° |
| b | 41.5 ± 0.1 Å | β | 92.1 ± 0.1° |
| c | 14.2 ± 0.1 Å | γ | 90°. |

26. The substantially crystalline Compound I methanol solvate (wet) according to any one of Embodiments 9-25, characterized by a ¹³C SSNMR spectrum having one or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

27. The substantially crystalline Compound I methanol solvate (wet) according to any one of Embodiments 9-26, characterized by a ¹³C SSNMR spectrum having two or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

28. The substantially crystalline Compound I methanol solvate (wet) according to any one of Embodiments 9-27, characterized by a ¹³C SSNMR spectrum having three or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

29. The substantially crystalline Compound I methanol solvate (wet) according to any one of Embodiments 9-28, characterized by a ¹³C SSNMR spectrum having four or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

30. The substantially crystalline Compound I methanol solvate (wet) according to any one of Embodiments 9-29, characterized by a ¹³C SSNMR spectrum having five or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

31. The substantially crystalline Compound I methanol solvate (wet) according to any one of Embodiments 9-30, characterized by a ¹³C SSNMR spectrum having six or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

32. The substantially crystalline Compound I methanol solvate (wet) according to any one of Embodiments 9-31, characterized by a ¹³C SSNMR spectrum having signals at 145.6±0.2 ppm and 132.5±0.2 ppm.

33. The substantially crystalline Compound I methanol solvate (wet) according to any one of Embodiments 9-31, characterized by a ¹³C SSNMR spectrum having signals at 145.6±0.2 ppm, 132.5±0.2 ppm, and 113.1±0.2 ppm.

34. The substantially crystalline Compound I methanol solvate (wet) according to any one of Embodiments 9-31, characterized by a ¹³C SSNMR spectrum having signals at 145.6±0.2 ppm, 132.5±0.2 ppm, 113.1±0.2 ppm, and 73.5±0.2 ppm.

35. The substantially crystalline Compound I methanol solvate (wet) according to any one of Embodiments 9-31, characterized by a ¹³C SSNMR spectrum having signals at 145.6±0.2 ppm, 132.5±0.2 ppm, 113.1±0.2 ppm, 73.5±0.2 ppm, and 55.9±0.2 ppm.

36. The substantially crystalline Compound I methanol solvate (wet) according to any one of Embodiments 9-31, characterized by a ¹³C SSNMR spectrum having signals at 145.6±0.2 ppm, 132.5±0.2 ppm, 113.1±0.2 ppm, 73.5±0.2 ppm, 55.9±0.2 ppm, and 35.2±0.2 ppm.

37. The substantially crystalline Compound I methanol solvate (wet) according to any one of Embodiments 9-31, characterized by a ¹³C SSNMR spectrum having signals at 73.5±0.2 ppm and 55.9±0.2 ppm.

38. The substantially crystalline Compound I methanol solvate (wet) according to any one of Embodiments 9-31, characterized by a ¹³C SSNMR spectrum having signals at 73.5±0.2 ppm, 55.9±0.2 ppm and 24.8±0.2 ppm.

39. The substantially crystalline Compound I methanol solvate (wet) according to any one of Embodiments 9-31, characterized by a ¹³C SSNMR spectrum having signals at 132.5±0.2 ppm, 73.5±0.2 ppm, 55.9±0.2 ppm, and 24.8±0.2 ppm.

40. The substantially crystalline Compound I methanol solvate (wet) according to any one of Embodiments 9-31, characterized by a ¹³C SSNMR spectrum having signals at 132.5±0.2 ppm, 113.1±0.2 ppm, 73.5±0.2 ppm, 55.9±0.2 ppm, and 24.8±0.2 ppm.

41. The substantially crystalline Compound I methanol solvate (wet) according to any one of Embodiments 9-40, characterized by a ¹³C SSNMR spectrum having signals at 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

42. The substantially crystalline Compound I methanol solvate (wet) according to any one of Embodiments 9-41, characterized by a ¹³C SSNMR spectrum substantially similar to FIG. 4.

43. The substantially crystalline Compound I methanol solvate (wet) according to any one of Embodiments 9-42, characterized by a ¹⁹F MAS having one or more signals selected from −63.9±0.2 ppm, −76.6±0.2 ppm, and −79.7±0.2 ppm.

44. The substantially crystalline Compound I methanol solvate (wet) according to any one of Embodiments 9-43, characterized by a ¹⁹F MAS having two or more signals selected from −63.9±0.2 ppm, −76.6±0.2 ppm, and −79.7±0.2 ppm.

45. The substantially crystalline Compound I methanol solvate (wet) according to any one of Embodiments 9-44, characterized by a ¹⁹F MAS having signals at −63.9±0.2 ppm, −76.6±0.2 ppm, and −79.7±0.2 ppm.

46. The substantially crystalline Compound I methanol solvate (wet) according to any one of Embodiments 9-45, characterized by a ¹⁹F MAS substantially similar to FIG. 5.

47. The substantially crystalline Compound I methanol solvate (wet) according to any one of Embodiments 9-46, prepared by (i) combining Compound I neat Form A, water and methanol in a sealed vial, (ii) heating to 65° C. and stirring until a homogeneous slurry is formed, and (iii) cooling the slurry without stirring and allowing to sit at room temperature over 3 days, to yield crystalline Compound I methanol solvate (wet).

48. Substantially crystalline Compound I methanol solvate (dry) (i.e., wherein less than 15% of Compound I is in amorphous form, wherein less than 10% of Compound I is in amorphous form, wherein less than 5% of Compound I is in amorphous form).

49. The substantially crystalline Compound I methanol solvate (dry) according to Embodiment 48, wherein Compound I methanol solvate (dry) is 100% crystalline.

50. The substantially crystalline Compound I methanol solvate (dry) according to Embodiment 48 or Embodiment 49, characterized by an X-ray powder diffractogram having signals at one or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

51. The substantially crystalline Compound I methanol solvate (dry) according to any one of Embodiments 48-50, characterized by an X-ray powder diffractogram having signals at two or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

52. The substantially crystalline Compound I methanol solvate (dry) according to any one of Embodiments 48-51, characterized by an X-ray powder diffractogram having signals at three or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

53. The substantially crystalline Compound I methanol solvate (dry) according to any one of Embodiments 48-52, characterized by an X-ray powder diffractogram having signals at four or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

54. The substantially crystalline Compound I methanol solvate (dry) according to any one of Embodiments 48-53, characterized by an X-ray powder diffractogram having signals at five or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

55. The substantially crystalline Compound I methanol solvate (dry) according to any one of Embodiments 48-54, characterized by an X-ray powder diffractogram having signals at six or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

56. The substantially crystalline Compound I methanol solvate (dry) according to any one of Embodiments 48-55, characterized by an X-ray powder diffractogram having signals at 25.9±0.2 degrees two-theta and 19.3±0.2 degrees two-theta.

57. The substantially crystalline Compound I methanol solvate (dry) according to any one of Embodiments 48-56, characterized by an X-ray powder diffractogram having signals at 19.3±0.2 degrees two-theta and 15.4±0.2 degrees two-theta.

58. The substantially crystalline Compound I methanol solvate (dry) according to any one of Embodiments 48-57, characterized by an X-ray powder diffractogram having signals at 25.9±0.2 degrees two-theta, 19.3±0.2 degrees two-theta, and 15.4±0.2 degrees two-theta.

59. The substantially crystalline Compound I methanol solvate (dry) according to any one of Embodiments 48-57, characterized by an X-ray powder diffractogram having signals at 19.3±0.2 degrees two-theta, 18.1±0.2 degrees two-theta, and 15.4±0.2 degrees two-theta.

60. The substantially crystalline Compound I methanol solvate (dry) according to any one of Embodiments 48-57, characterized by an X-ray powder diffractogram having signals at 25.9±0.2 degrees two-theta, 19.3±0.2 degrees two-theta, 18.1+0.2 degrees two-theta, and 15.4±0.2 degrees two-theta.

61. The substantially crystalline Compound I methanol solvate (dry) according to any one of Embodiments 48-57, characterized by an X-ray powder diffractogram having signals at 27.2±0.2 degrees two-theta, 26.4±0.2 degrees two-theta, 25.9+0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

62. The substantially crystalline Compound I methanol solvate (dry) according to any one of Embodiments 48-61, characterized by an X-ray powder diffractogram substantially similar to FIG. 6.

63. The substantially crystalline Compound I methanol solvate (dry) according to any one of Embodiments 48-62, prepared by (i) combining Compound I neat Form A, water and methanol in a sealed vial, (ii) heating to 65° C. and stirring until a homogeneous slurry is formed, (iii) cooling the slurry without stirring and allowing it to sit at room temperature over 3 days, and (iv) drying in a vacuum oven at 60° C. overnight to yield crystalline Compound I methanol solvate (dry).

64. Substantially crystalline Compound I p-toluene sulfonic acid (i.e., wherein less than 15% of Compound I is in amorphous form, wherein less than 10% of Compound I is in amorphous form, wherein less than 5% of Compound I is in amorphous form).

65. The substantially crystalline Compound I p-toluene sulfonic acid according to Embodiment 64, wherein Compound I p-toluene sulfonic acid is 100% crystalline.

66. The substantially crystalline Compound I p-toluene sulfonic acid according to Embodiment 64 or Embodiment 65, characterized by an X-ray powder diffractogram having signals at one or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9+0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

67. The substantially crystalline Compound I p-toluene sulfonic acid according to any one of embodiments 64-66, characterized by an X-ray powder diffractogram having signals at two or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

68. The substantially crystalline Compound I p-toluene sulfonic acid according to any one of embodiments 64-67, characterized by an X-ray powder diffractogram having signals at three or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

69. The substantially crystalline Compound I p-toluene sulfonic acid according to any one of embodiments 64-68, characterized by an X-ray powder diffractogram having signals at four or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

70. The substantially crystalline Compound I p-toluene sulfonic acid according to any one of embodiments 64-69, characterized by an X-ray powder diffractogram having signals at five or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

71. The substantially crystalline Compound I p-toluene sulfonic acid according to any one of embodiments 64-70, characterized by an X-ray powder diffractogram having signals at six or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

72. The substantially crystalline Compound I p-toluene sulfonic acid according to any one of embodiments 64-71, characterized by an X-ray powder diffractogram having signals at 5.7±0.2 degrees two-theta and 5.8±0.2 degrees two-theta.

73. The substantially crystalline Compound I p-toluene sulfonic acid according to any one of embodiments 64-71, characterized by an X-ray powder diffractogram having signals at 5.7±0.2 degrees two-theta, 5.8±0.2 degrees two-theta, and 7.4±0.2 degrees two-theta.

74. The substantially crystalline Compound I p-toluene sulfonic acid according to any one of embodiments 64-71, characterized by an X-ray powder diffractogram having signals at 0.7±0.2 degrees two-theta, 5.8±0.2 degrees two-theta, 7.4±0.2 degrees two-theta, and 10.1±0.2 degrees two-theta.

75. The substantially crystalline Compound I p-toluene sulfonic acid according to any one of embodiments 64-71, characterized by an X-ray powder diffractogram having signals at 5.7±0.2 degrees two-theta, 5.8±0.2 degrees two-theta, 7.4±0.2 degrees two-theta, 10.1±0.2 degrees two-theta, and 11.5±0.2 degrees two-theta.

76. The substantially crystalline Compound I p-toluene sulfonic acid according to any one of embodiments 64-71, characterized by an X-ray powder diffractogram having signals at 5.7±0.2 degrees two-theta, 5.8±0.2 degrees two-theta, 7.4±0.2 degrees two-theta, 10.1±0.2 degrees two-theta, 11.5±0.2 degrees two-theta, and 11.9±0.2 degrees two-theta.

77. The substantially crystalline Compound I p-toluene sulfonic acid according to any one of Embodiments 64-76, characterized by an X-ray powder diffractogram substantially similar to FIG. 9.

78. The substantially crystalline Compound I p-toluene sulfonic acid according to any one of Embodiments 64-77, characterized by a ¹³C SSNMR spectrum having one or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

79. The substantially crystalline Compound I p-toluene sulfonic acid according to any one of Embodiments 64-78, characterized by a ¹³C SSNMR spectrum having two or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

80. The substantially crystalline Compound I p-toluene sulfonic acid according to any one of Embodiments 64-79, characterized by a ¹³C SSNMR spectrum having three or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

81. The substantially crystalline Compound I p-toluene sulfonic acid according to any one of Embodiments 64-80, characterized by a ¹³C SSNMR spectrum having four or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

82. The substantially crystalline Compound I p-toluene sulfonic acid according to any one of Embodiments 64-81, characterized by a ¹³C SSNMR spectrum having five or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

83. The substantially crystalline Compound I p-toluene sulfonic acid according to any one of Embodiments 64-82, characterized by a ¹³C SSNMR spectrum having six or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

84. The substantially crystalline Compound I p-toluene sulfonic acid according to any one of Embodiments 64-83, characterized by a ¹³C SSNMR spectrum having signals at 141.0±0.2 ppm and 19.5±0.2 ppm.

85. The substantially crystalline Compound I p-toluene sulfonic acid according to any one of Embodiments 64-82, characterized by a ¹³C SSNMR spectrum having 141.0±0.2 ppm, 23.0±0.2 ppm, and 19.5±0.2 ppm.

86. The substantially crystalline Compound I p-toluene sulfonic acid according to any one of Embodiments 64-82, characterized by a ¹³C SSNMR spectrum having 141.0±0.2 ppm, 57.0±0.2 ppm, 23.0±0.2 ppm, and 19.5±0.2 ppm.

87. The substantially crystalline Compound I p-toluene sulfonic acid according to any one of Embodiments 64-82, characterized by a ¹³C SSNMR spectrum having 141.0±0.2 ppm, 57.0±0.2 ppm, 25.0±0.2 ppm, 23.0±0.2 ppm, and 19.5±0.2 ppm.

88. The substantially crystalline Compound I p-toluene sulfonic acid according to any one of Embodiments 64-82, characterized by a ¹³C SSNMR spectrum having 141.0±0.2 ppm, 57.0±0.2 ppm, 31.0±0.2 ppm, 25.0±0.2 ppm, 23.0±0.2 ppm, and 19.5±0.2 ppm.

89. The substantially crystalline Compound I p-toluene sulfonic acid according to any one of Embodiments 64-82, characterized by a ¹³C SSNMR spectrum having 141.0±0.2 ppm, 126.7±0.2 ppm, 57.0±0.2 ppm, 31.0±0.2 ppm, 25.0±0.2 ppm, 23.0 0.2 ppm, and 19.5±0.2 ppm.

90. The substantially crystalline Compound I p-toluene sulfonic acid according to any one of Embodiments 64-89, characterized by a ¹³C SSNMR spectrum substantially similar to FIG. 11.

91. The substantially crystalline Compound I p-toluene sulfonic acid according to any one of Embodiments 64-90, characterized as having a ¹⁹F MAS with one or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

92. The substantially crystalline Compound I p-toluene sulfonic acid according to any one of Embodiments 64-90, characterized as having a ¹⁹F MAS with two or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

93. The substantially crystalline Compound I p-toluene sulfonic acid according to any one of Embodiments 64-90, characterized as having a ¹⁹F MAS with three or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

94. The substantially crystalline Compound I p-toluene sulfonic acid according to any one of Embodiments 64-90, characterized as having a ¹⁹F MAS with four or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

95. The substantially crystalline Compound I p-toluene sulfonic acid according to any one of Embodiments 64-90, characterized as having a ¹⁹F MAS with five or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

96. The substantially crystalline Compound I p-toluene sulfonic acid according to any one of Embodiments 64-90, characterized as having a ¹⁹F MAS with six or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

97. The substantially crystalline Compound I p-toluene sulfonic acid according to any one of Embodiments 64-90, characterized as having a ¹⁹F MAS with signals at −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm

98. The substantially crystalline Compound I p-toluene sulfonic acid according to any one of Embodiments 64-97, characterized by a ¹⁹F MAS substantially similar to FIG. 12.

99. The substantially crystalline Compound I methanol solvate (wet) according to any one of Embodiments 64-98, prepared by (i) adding p-toluene sulfonic acid to Compound I neat Form A in a milling ball tube, (ii) adding methanol and water (60:40 v/v), (iii) ball milling at 7500 rpm for 3 cycles of 60 seconds with 10 second pauses, and (iv) drying the resulting material in a vacuum dry oven at 40° C., to yield crystalline Compound I p-toluene sulfonic acid.

100. A pharmaceutical composition comprising Compound I according to any one of Embodiments 1-99 and a pharmaceutically acceptable carrier

101. The pharmaceutical composition according to Embodiment 100 further comprising one or more additional thereapeutic agents.

102. The pharmaceutical composition according to Embodiment 101, wherein the pharmaceutical composition comprises one or more additional CFTR modulating compounds.

103. The pharmaceutical composition according to Embodiment 101 or Embodiment 102, wherein the pharmaceutical composition comprises one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof.

104. The Compound I according to any one of Embodiments 1-99, or the pharmaceutical composition according to any one of Embodiments 100-103, for use in the treatment of cystic fibrosis.

105. Use of the Compound I according to any one of Embodiments 1-99, or the pharmaceutical composition according to any one of Embodiments 100-103, in the manufacture of a medicament for the treatment of cystic fibrosis.

106. A method of treating cystic fibrosis comprising administering the Compound I according to any one of Embodiments 1-99, or the pharmaceutical composition according to any one of Embodiments 101-103, to a subject in need thereof.

### B. Set 2

1. A method for preparing Compound I:

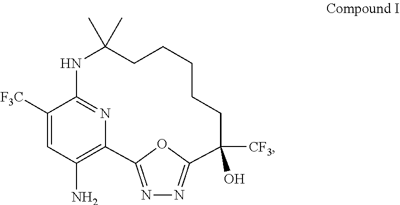 [SMILES: CC1(C)CCCCC[C@](O)(C(F)(F)F)C2=NN=C(O2)c2nc(c(C(F)(F)F)cc2N)N1]

or a stereoisomer of Compound I, or a deuterated derivative of Compound I or a stereoisomer thereof, or pharmaceutically acceptable salts of any of the foregoing, wherein the method comprises converting a compound of Formula I:

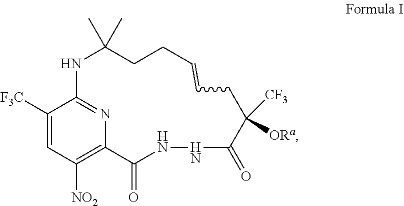

or a stereoisomer of the compound of Formula I, or a deuterated derivative of the compound of Formula I or a stereoisomer thereof, or salts of any of the foregoing, into Compound I, or a stereoisomer thereof, or a deuterated derivative of Compound I or a stereoisomer thereof, or salts of any of the foregoing, wherein R^{a} is selected from alcohol protecting groups.

2. The method according to Embodiment 1, wherein R^{a} is benzyl (Bn).

3. A method for preparing Compound I:

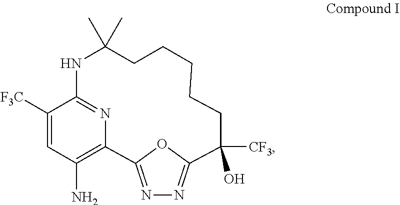 [SMILES: CC1(C)CCCCC[C@](O)(C(F)(F)F)C2=NN=C(O2)c2nc(c(C(F)(F)F)cc2N)N1]

or a stereoisomer of Compound I, or a deuterated derivative of Compound I or a stereoisomer thereof, or pharmaceutically acceptable salts of any of the foregoing, wherein the method comprises converting a compound of Formula II.

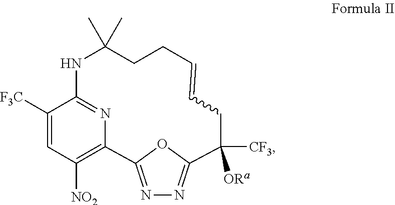

or a stereoisomer of the compound of Formula II, or a deuterated derivative of the compound of Formula II or a stereoisomer thereof, or salts of any of the foregoing, into Compound I, or a stereoisomer thereof, or a deuterated derivative of Compound I or a stereoisomer thereof, or salts of any of the foregoing, wherein R^{a} is selected from alcohol protecting groups, and with the proviso that R^{a} is not benzyl (Bn).

4. The method according to Embodiment 3, wherein R^{a} is selected from naphthylmethyl, biphenylmethyl, acetyl (Ac), trifluoroacetyl, and benzoyl (Bz).

5. The method according to Embodiment 3 or 4, wherein converting the compound of Formula II, or a stereoisomer of the compound of Formula II, or a deuterated derivative of the compound of Formula II or a stereoisomer thereof, or salts of any of the foregoing, into Compound I, or a stereoisomer thereof, or a deuterated derivative of Compound I or a stereoisomer thereof, or salts of any of the foregoing, is performed in the presence of reducing reaction conditions.

6. The method according to Embodiment 5, wherein the reducing conditions are selected from hydrogen gas (H₂), palladium on carbon (Pd/C), and methanolic ammonia (NH₃/MeOH); hydrogen gas (H₂), palladium on carbon (Pd/C), and ethanolic ammonia (NH₃/EtOH); hydrogen gas (H₂), palladium on alumina (Pd/Al), and methanolic ammonia (NH₃/MeOH); hydrogen gas (H₂), palladium on alumina (Pd/Al), and methanolic ammonia (NH₃/EtOH); hydrogen gas (H₂), platinum on carbon (Pt/C), and methanolic ammonia (NH₃/MeOH); hydrogen gas (H₂), platinum on carbon (Pt/C), and ethanolic ammonia (NH₃/EtOH); hydrogen gas (H₂), platinum on alumina (Pt/Al), and methanolic ammonia (NH₃/MeOH); hydrogen gas (H₂), platinum on alumina (Pt/Al), and methanolic ammonia (NH₃/EtOH); hydrogen gas (H₂), ruthenium on carbon (Ru/C), and methanolic ammonia (NH₃/MeOH); hydrogen gas (H₂), ruthenium on carbon (Ru/C), and ethanolic ammonia (NH₃/EtOH); hydrogen gas (H₂), ruthenium on alumina (Ru/Al), and methanolic ammonia (NH₃/MeOH); and hydrogen gas (H₂), ruthenium on alumina (Ru/Al), and methanolic ammonia (NH₃/EtOH).

7. The method according to Embodiment 5 or 6, wherein the reducing conditions are hydrogen gas (H₂), palladium on carbon (Pd/C), and methanolic ammonia (NH₃/MeOH).

8. The method according to any one of Embodiments 1 to 7, wherein the method comprises converting the compound of Formula I.

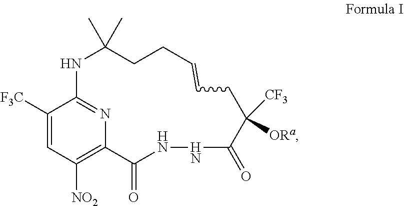

or a stereoisomer of the compound of Formula I, or a deuterated derivative of the compound of Formula I or a stereoisomer thereof, or salts of any of the foregoing, into the compound of Formula II, or a stereoisomer thereof, or a deuterated derivative of the compound of Formula II or a stereoisomer thereof, or salts of any of the foregoing, wherein R^{a} is selected from alcohol protecting groups.

9. The method according to Embodiment 8, wherein R^{a} is benzyl (Bn).

10. The method according to Embodiment 8 or 9, wherein converting the compound of Formula I, or a stereoisomer of the compound of Formula I, or a deuterated derivative of the compound of Formula I or a stereoisomer thereof, or salts of any of the foregoing, into the compound of Formula II, or a stereoisomer thereof, or a deuterated derivative of the compound of Formula II or a stereoisomer thereof, or salts of any of the foregoing, is performed in the presence of a dehydrating reagent and a base.

11. The method according to Embodiment 10, wherein the dehydrating reagent is selected from 2-chloro-1,3-dimethylimidazolinium chloride (DMC), p-toluenesulfonyl chloride (p-TsCl), 2-chloro-4,6-dimethoxy-1,3,5-triazine (CDMT), propylphosphonic anhydride (T3P), 1,1′-carbonyldiimidazole (CDI), and 1-[bis(dimethylamino)methylene]-1H-1,2,3-triazolo[4,5-b]pyridinium 3-oxid hexafluorophosphate (HATU).

12. The method according to Embodiment 10 or 11, wherein the dehydrating reagent is 2-chloro-1,3-dimethylimidazolinium chloride (DMC).

13. The method according to any one of Embodiments 10 to 12, wherein the base is selected from 1,4-diazabicyclo[2.2.2]octane (DABCO), triethylamine (Et₃N), N-methylmorpholine (NMM), pyridine, and DIPEA (N,N-diisopropylethylamine).

14. The method according to any one of Embodiments 10 to 13, wherein the base is 1,4-diazabicyclo[2.2.2]octane (DABCO).

15. The method according to any one of Embodiments 10 to 14, wherein the dehydrating reagent is 2-chloro-1,3-dimethylimidazolinium chloride (DMC) and the base is 1,4-diazabicyclo[2.2.2]octane (DABCO).

16. The method according to any one of Embodiments 8 to 15, wherein the method comprises converting a compound of Formula III:

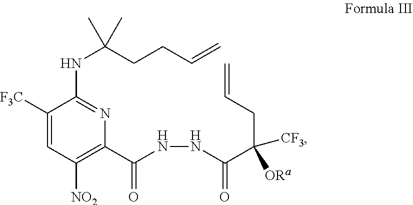

or a stereoisomer of the compound of Formula III, or a deuterated derivative of the compound of Formula III or a stereoisomer thereof, or salts of any of the foregoing, into the compound of Formula I, or a stereoisomer thereof, or a deuterated derivative of the compound of Formula I or a stereoisomer thereof, or salts of any of the foregoing, wherein R^{a} is selected from alcohol protecting groups.

17. The method according to Embodiment 16, wherein R^{a} is benzyl (Bn).

18. The method according to Embodiment 16 or 17, wherein converting the compound of Formula III, or a stereoisomer of the compound of Formula III, or a deuterated derivative of the compound of Formula III or a stereoisomer thereof, or salts of any of the foregoing, into the compound of Formula I, or a stereoisomer thereof, or a deuterated derivative of the compound of Formula I or a stereoisomer thereof, or salts of any of the foregoing, is performed in the presence of a ruthenium catalyst.

19. The method according to Embodiment 18, wherein the ruthenium catalyst is selected from dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zahn 1B catalyst); dichloro[1,3-bis(2,4,6-trimethylphenyl)-2-imidazolidinylidene](2-isopropoxyphenylmethylene)ruthenium(II); dichloro[1,3-bis(2,6-isopropylphenyl)-2-imidazolidinylidene](2-isopropoxyphenylmethylene)ruthenium(II); (1,3-dimesitylimidazolidin-2-ylidene)dichloro(2-isopropoxy-5-nitrobenzylidene)ruthenium(II); dichloro(3-phenyl-1H-inden-1-ylidene)bis(tricyclohexylphosphine)ruthenium(II); dichloro[1,3-bis(2,4,6-trimethylphenyl)-2-imidazolidinylidene](3-phenyl-1H-inden-1-ylidene)(tricyclohexylphosphine)ruthenium(II); and dichloro(2-isopropoxyphenylmethylene) (tricyclohexylphosphine)ruthenium(II).

20. The method according to Embodiment 18 or 19, wherein the ruthenium catalyst is dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zahn 1B catalyst).

21. The method according to any one of Embodiments 16 to 20, wherein the method comprises reacting Compound 2:

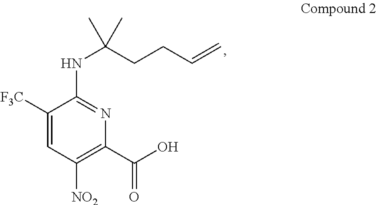 [SMILES: C=CCCC(C)(C)Nc1nc(C(=O)O)c([N+](=O)[O-])cc1C(F)(F)F]

or a deuterated derivative of Compound 2, or a salt of any of the foregoing, with a compound of Formula IV:

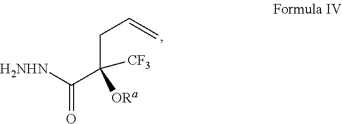

or a stereoisomer of the compound of Formula IV, or a deuterated derivative of the compound of Formula IV or a stereoisomer thereof, or salts of any of the foregoing, to produce the compound of Formula III, or a stereoisomer thereof, or a deuterated derivative of the compound of Formula III or a stereoisomer thereof, or salts of any of the foregoing, wherein R^{a} is selected from alcohol protecting groups.

22. The method according to Embodiment 21, wherein R^{a} is benzyl (Bn).

23. The method according to Embodiment 21 or 22, wherein reacting Compound 2, or a deuterated derivative of Compound 2, or a salt of any of the foregoing, with a compound of Formula IV, or a stereoisomer of the compound of Formula IV, or a deuterated derivative of the compound of Formula IV or a stereoisomer thereof, or salts of any of the foregoing, is performed in the presence of a peptide coupling agent and a base.

24. The method according to Embodiment 23, wherein the peptide coupling agent is selected from 2-chloro-4,6-dimethoxy-1,3,5-triazine (CDMT), 1,1-carbonyl diimidazole (CDI), 2-chloro-1-methylpyridinium iodide (CMPI), 1-[(1-(cyano-2-ethoxy-2-oxoethylideneaminooxy)dimethylamino-morpholinomethylene)]methanaminium hexafluorophosphate (COMU), isobutyl chloroformate (IBCF), and propanephosphonic acid anhydride (T3P).

25. The method according to Embodiment 23 or 24, wherein the peptide coupling agent is 2-chloro-4,6-dimethoxy-1,3,5-triazine (CDMT).

26. The method according to any one of Embodiments 23 to 25, wherein the base is selected from N-methylmorpholine (NMM), 1-methylimidazole, triethylamine (Et₃N), N,N,-diisopropylethylamine (DIPEA), and pyridine.

27. The method according to any one of Embodiments 23 to 26, wherein the base is N-methylmorpholine (NMM).

28. The method according to any one of Embodiments 23 to 27, wherein the dehydrating reagent is 2-chloro-4,6-dimethoxy-1,3,5-triazine (CDMT) and the base is N-methylmorpholine (NMM).

29. The method according to any one of Embodiments 21 to 28, wherein the method comprises converting a compound of Formula V:

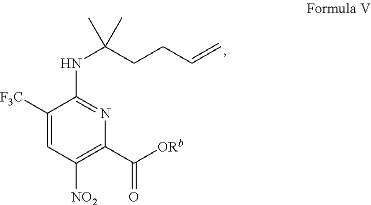

or a deuterated derivative of the compound of Formula V, or a salt of any of the foregoing, into Compound 2, or a deuterated derivative of Compound 2, or a salt of any of the foregoing, wherein R^{b} is selected from methyl (Me), ethyl (Et), n-propyl (n-Pr), isopropyl (i-Pr), and tert-butyl (t-Bu).

30. The method according to Embodiment 29, wherein R^{b} is methyl (Me).

31. The method according to Embodiment 29 or 30, wherein converting the compound of Formula V, or a deuterated derivative of the compound of Formula V, or a salt of any of the foregoing, into Compound 2, or a deuterated derivative of Compound 2, or a salt of any of the foregoing, is performed in the presence of an aqueous hydroxide base.

32. The method according to Embodiment 31, wherein the hydroxide base is selected from lithium hydroxide (LiOH), sodium hydroxide (NaOH), potassium hydroxide (KOH), and cesium hydroxide (CsOH).

33. The method according to Embodiment 31 or 32, wherein the base is sodium hydroxide (NaOH).

34. The method according to any one of Embodiments 29 to 33, wherein the method comprises reacting a compound of Formula VI:

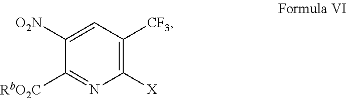

or a deuterated derivative of a compound of Formula VI, or a salt of any of the foregoing, with Compound 3:

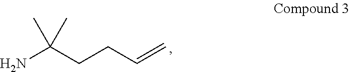

or a deuterated derivative of a compound of Formula VI, or a salt of any of the foregoing, to produce the compound of Formula V, or a deuterated derivative of the compound of Formula V, or a salt of any of the foregoing, wherein:
R^{b} is selected from methyl (Me), ethyl (Et), n-propyl (n-Pr), isopropyl (i-Pr), and tert-butyl (t-Bu); and
X is selected from Cl and Br.

35. The method according to Embodiment 34, wherein R^{b} is methyl (Me) and X is Cl.

36. The method according to Embodiment 34 or 35, wherein reacting the compound of Formula VI, or a deuterated derivative of the compound of Formula IV, or a salt of any of the foregoing, with compound 3, or a deuterated derivative of Compound 3, or a salt of any of the foregoing, is performed in the presence of a base.

37. The method according to Embodiment 36, wherein the base is selected from sodium carbonate (NaHCO₃), N,N,-diisopropylethylamine (DIPEA), N-methylmorpholine (NMM), sodium bicarbonate (NaHCO₃), potassium bicarbonate (KHCO₃), potassium carbonate (K₂C₀₃), and potassium phosphate dibasic (K₂HPO₄).

38. The method according to any one of Embodiments 36 or 37, wherein the base is N,N,-diisopropylethylamine (DIPEA).

39. The method according to any one of Embodiments 34 to 38, wherein the method comprises converting a compound of Formula VII:

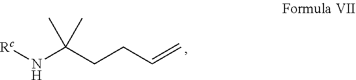

or a deuterated derivative of the compound of Formula VII, or a salt of any of the foregoing, into Compound 3, or a deuterated derivative of Compound 3, or a salt of any of the foregoing, wherein R^{c} is selected from amine protecting groups.

40. The method according to Embodiment 39, wherein R^{c} is tert-butyloxycarbonyl (Boc).

41. The method according to Embodiment 39 or 40, wherein converting the compound of Formula VII, or a deuterated derivative of the compound of Formula VII, or a salt of any of the foregoing, into Compound 3, or a deuterated derivative of Compound 3, or a salt of any of the foregoing, is performed in the presence of a protic acid.

42. The method according to Embodiment 41, wherein the protic acid is selected from hydrochloric acid (HCl), trifluoroacetic acid (TFA), p-toluenesulfonic acid (pTsOH), and methanesulfonic acid (MsOH).

43. The method according to Embodiment 41 or 42, wherein the protic acid is hydrochloric acid (HCl).

44. The method according to any one of Embodiments 39 to 43, wherein the method comprises converting a comppund of Formula VII:

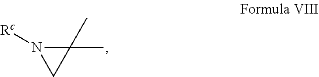

or a deuterated derivative of the compound of Formula VIII, into the compound of Formula VII, or a deuterated derivative of the compound of Formula VII, or a salt of any of the foregoing, wherein R^{c} is selected from amine protecting groups.

45. The method according to Embodiment 44, wherein R^{c} is tert-butyloxycarbonyl (Boc).

46. The method according to Embodiment 44 or 45, wherein converting the compound of Formula VIII, or a deuterated derivative of the compound of Formula VIII, into the compound of Formula VII, or a deuterated derivative of the compound of Formula VII, or a salt of any of the foregoing, is performed in the presence of an allylmagnesium halide and a copper(I) halide.

47. The method according to Embodiment 46, wherein the allylmagnesium halide is selected from allylmagnesium chloride and allylmagnesium bromide.

48. The method according to Embodiment 46 or 47, wherein the allylmagnesium halide is allyl magnesium chloride.

49. The method according to any one of Embodiments 46 to 48, wherein the copper(I) halide is selected from copper(I) chloride, copper(I) bromide, copper(I) bromide dimethyl sulfide complex, and copper(I) iodide.

50. The method according to any one of Embodiments 46 to 49, wherein the copper(I) halide is copper(I) bromide dimethyl sulfide complex.

51. The method according to any one of Embodiments 46 to 50, wherein the allylmagnesium halide is allyl magnesium chloride and the copper(I) halide is copper(I) bromide dimethyl sulfide complex.

52. The method according to any one of Embodiments 44 to 51, wherein the method comprises converting a compound of Formula IX:

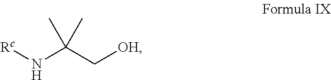

or a deuterated derivative of the compound of Formula IX or a salt of any of the foregoing, into the compound of Formula VIII, or a deuterated derivative of the compound of Formula VIII, or a salt of any of the foregoing, wherein R is selected from amine protecting groups.

53. The method according to Embodiment 52, wherein R is tert-butyloxycarbonyl (Boc).

54. The method according to Embodiment 52 or 53, wherein converting the compound of Formula IX, or a deuterated derivative of the compound of Formula IX, or a salt of any of the foregoing, into the compound of Formula VIII, or a deuterated derivative of the compound of Formula VIII, or a salt of any of the foregoing, is performed in the presence of a sulfonyl halide and a base.

55. The method according to Embodiment 54, wherein the sulfonyl halide is selected from
p-toluenesulfonyl chloride (p-TsCl), benzenesulfonylchloride, and methanesulfonyl chloride (MsCl).

56. The method according to Embodiment 54 or 55, wherein the sulfonyl halide isp-toluenesulfonyl chloride (p-TsCl).

57. The method according to any one of Embodiments 54 to 56, wherein the base is selected from sodium hydroxide (NaOH), potassium hydroxide (KOH), and lithium hydroxide (LiOH).

58. The method according to any one of Embodiments 54 to 57, wherein the base is potassium hydroxide (KOH).

59. The method according to any one of Embodiments 54 to 58, wherein the sulfonyl halide is
p-toluenesulfonyl chloride (p-TsCl) and the base is potassium hydroxide (KOH).

60. The method according to any one of Embodiments 52 to 59, wherein the method comprises converting a compound of Formula X:

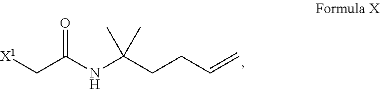

or a deuterated derivative of the compound of Formula X, or a salt of any of the foregoing, into Compound 3, or a deuterated derivative of Compound 3, or a salt of any of the foregoing, wherein X¹ is selected from F, Cl, and Br.

61. The method according to Embodiment 60, wherein X¹ is Cl.

62. The method according to Embodiment 60 or 61, wherein converting the compound of Formula X, or a deuterated derivative of the compound of Formula X, or a salt of any of the foregoing, into Compound 3, or a deuterated derivative of Compound 3, or a salt of any of the foregoing, is performed in the presence of thiourea and a protic acid.

63. The method according to Embodiment 62, wherein the protic acid is selected from hydrochloric acid (HCl), acetic acid (AcOH), and sulfuric acid (H₂SO₄).

64. The method according to Embodiment 62 or 63, wherein the protic acid is hydrochloric acid (HCl).

65. The method according to any one of Embodiments 60 to 64, wherein the method comprises converting hex-5-en-2-one:

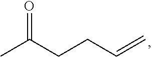

or a deuterated derivative of hex-5-en-2-one, or a salt of any of the foregoing, into the compound of Formula X, or a deuterated derivative of the compound of Formula X, or a salt of any of the foregoing, wherein X¹ is selected from F, Cl, and Br.

66. The method according to Embodiment 65, wherein the method for converting hex-5-en-2-one, or a deuterated derivative of hex-5-en-2-one, or a salt of any of the foregoing, into the compound of Formula X, or a deuterated derivative of the compound of Formula X, or a salt of any of the foregoing, comprises:
(i) reacting hex-5-en-2-one, or a deuterated derivative of hex-5-en-2-one, or a salt of any of the foregoing, with a methylmagnesium halide, or a deuterated derivative of the methylmagnesium halide, or a salt of any of the foregoing; and
(ii) reacting the product of step (i) with a 2-haloacetonitrile or a deuterated derivative of the 2-haloacetonitrile
to produce a compound of Formula X, or a salt of any of the foregoing, wherein X¹ is selected from F, Cl, and Br.

67. The method according to Embodiment 66, wherein the methylmagnesium halide is selected from methylmagnesium chloride (MeMgCl), methylmagnesium bromide (MeMgBr), and methylmagnesium iodide (MeMgI).

68. The method according to Embodiment 66 or 67, wherein the methylmagnesium halide is methylmagnesium chloride (MeMgCl).

69. The method according to any one of Embodiments 66 to 68, wherein the 2-haloacetonitrile is selected from 2-fluoroacetonitrile, 2-chloroacetonitrile, and 2-bromoacetonitrile.

70. The method according to any one of Embodiments 66 to 69, wherein X¹ is Cl and the 2-haloacetonitrile is 2-chloroacetonitrile.

71. The method according to any one of Embodiments 66 to 70, wherein X¹ is Cl, the methylmagnesium halide is methylmagnesium chloride (MeMgCl) and the 2-haloacetonitrile is 2-chloroacetonitrile.

72. A method for preparing Compound I:

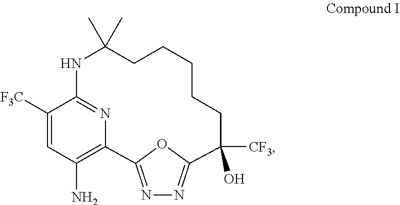 [SMILES: CC1(C)CCCCC[C@](O)(C(F)(F)F)C2=NN=C(O2)c2nc(c(C(F)(F)F)cc2N)N1]

or a stereoisomer of Compound I, or a deuterated derivative of Compound I or a stereoisomer thereof, or pharmaceutically acceptable salts of any of the foregoing, wherein the method comprises converting a compound of Formula I, Formula II, or Formula III:

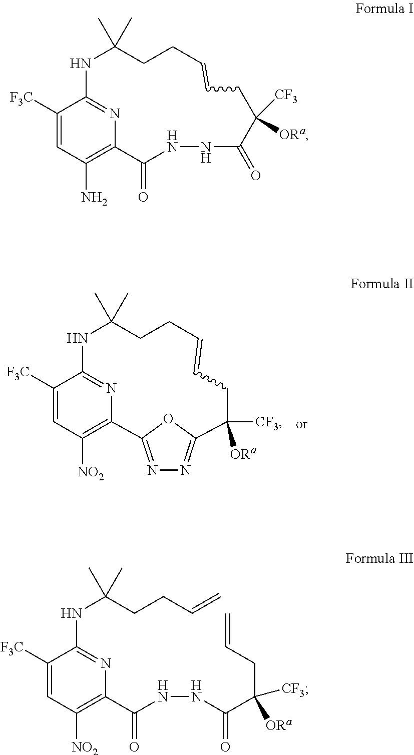

or a stereoisomer of the compound, or a deuterated derivative of the compound or a stereoisomer thereof, or a salt of any of the foregoing, into Compound I, or a stereoisomer of Compound I, or a deuterated derivative of Compound I or a stereoisomer thereof, or a pharmaceutically acceptable salt of any of the foregoing, wherein:
   R^{a} is selected from alcohol protecting groups; and
   the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (E/Z mixture); and pharmaceutically acceptable salts thereof.

73. The method according to Embodiment 72, wherein R^{a} is benzyl (Bn).

74. A compound selected from:

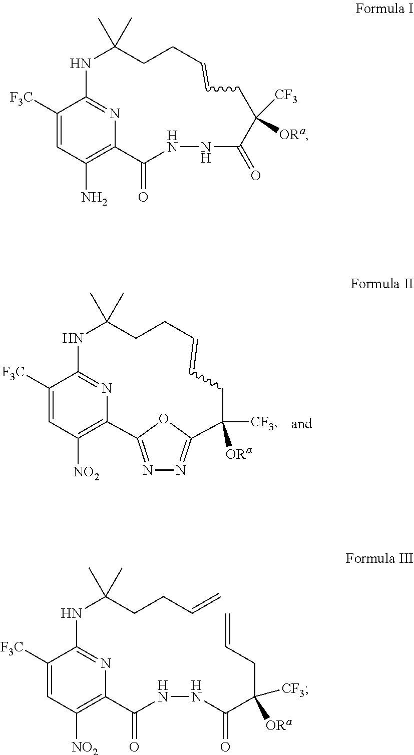

or a stereoisomer of the compound, or a deuterated derivative of the compound or a stereoisomer thereof, or a salt of any of the foregoing,
wherein:
   R^{a} is selected from alcohol protecting groups; and
   the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (E/Z mixture);
and pharmaceutically acceptable salts thereof.

75. A compound selected from:

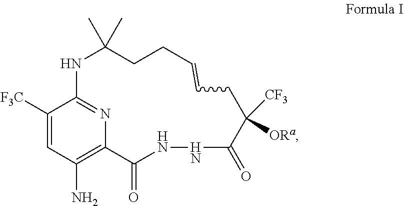

or a stereoisomer of the compound, or a deuterated derivative of the compound or a stereoisomer thereof, or a salt of any of the foregoing, wherein R^{a} is selected from alcohol protecting groups.

76. The compound according to Embodiment 74 or 75, wherein R^{a} is benzyl (Bn).

77. A compound having the following formula:

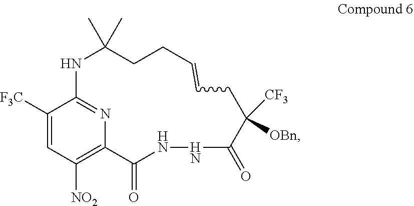 [SMILES: CC1(C)CCC=CC[C@](OCc2ccccc2)(C(F)(F)F)C(=O)NNC(=O)c2nc(c(C(F)(F)F)cc2[N+](=O)[O-])N1]

or a stereoisomer of the compound, or a deuterated derivative of the compound or a stereoisomer thereof, or a salt of any of the foregoing.

78. A method for preparing Compound I:

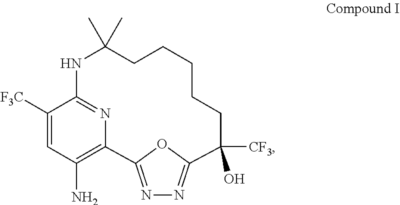 [SMILES: CC1(C)CCCCC[C@](O)(C(F)(F)F)C2=NN=C(O2)c2nc(c(C(F)(F)F)cc2N)N1]

or a stereoisomer of Compound I, or a deuterated derivative of Compound I or a stereoisomer thereof, or pharmaceutically acceptable salts of any of the foregoing, wherein the method comprises converting a compound of Formula II:

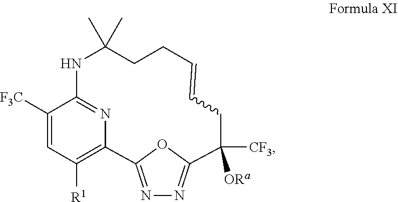

or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing,
wherein:
R^{a} is selected from alcohol protecting groups, and
R¹ is selected from —N(Boc)₂, —NHBoc, —N(Phth), —NH(Cbz), and —NO₂.

79. The method according to Embodiment 78, wherein R^{a} is benzyl (Bn).

80. The method according to Embodiment 78 or 79, wherein converting the compound of Formula XI, or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing, comprises a reaction that is performed in the presence of reducing reaction conditions.

81. The method according to Embodiment 80, wherein the reducing conditions are selected from hydrogen gas (H₂), palladium on carbon (Pd/C), and methanolic ammonia (NH₃/MeOH); hydrogen gas (H₂), palladium on carbon (Pd/C), and ethanolic ammonia (NH₃/EtOH); hydrogen gas (H₂), palladium on alumina (Pd/Al), and methanolic ammonia (NH₃/MeOH); hydrogen gas (H₂), palladium on alumina (Pd/Al), and methanolic ammonia (NH₃/EtOH); hydrogen gas (H₂), platinum on carbon (Pt/C), and methanolic ammonia (NH₃/MeOH); hydrogen gas (H₂), platinum on carbon (Pt/C), and ethanolic ammonia (NH₃/EtOH); hydrogen gas (H₂), platinum on alumina (Pt/Al), and methanolic ammonia (NH₃/MeOH); hydrogen gas (H₂), platinum on alumina (Pt/Al), and methanolic ammonia (NH₃/EtOH); hydrogen gas (H₂), ruthenium on carbon (Ru/C), and methanolic ammonia (NH₃/MeOH); hydrogen gas (H₂), ruthenium on carbon (Ru/C), and ethanolic ammonia (NH₃/EtOH); hydrogen gas (H₂), ruthenium on alumina (Ru/Al), and methanolic ammonia (NH₃/MeOH); and hydrogen gas (H₂), ruthenium on alumina (Ru/Al), and methanolic ammonia (NH₃/EtOH).

82. The method according to Embodiment 80 or 81, wherein the reducing conditions are hydrogen gas (H₂), palladium on carbon (Pd/C), and methanolic ammonia (NH₃/MeOH).

83. The method according to any one of Embodiments 78 to 82, wherein converting the compound of Formula XI, or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing, further comprises a reaction that is performed in the presence of an acid.

84. The method according to Embodiment 83, wherein the acid is selected from trifluoroacetic acid (TFA), hydrochloric acid (HCl), methanesulfonic acid (MsOH), phosphoric acid (H₃PO₄), and sulfuric acid (H₂SO₄).

85. The method according to Embodiment 83 or 84, wherein the acid is trifluoroacetic acid (TFA).

86. The method according to any one of Embodiments 78 to 85, wherein the method comprises converting the compound of Formula XII.

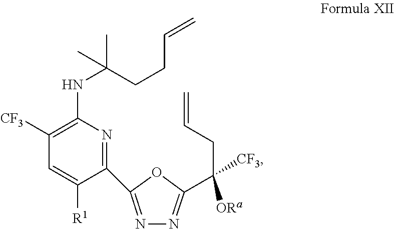

or a stereoisomer of the compound of Formula XII, or a deuterated derivative of the compound of Formula XII or a stereoisomer thereof, or salts of any of the foregoing, into the compound of Formula XI, or a stereoisomer thereof, or a deuterated derivative of the compound of Formula XI or a stereoisomer thereof, or salts of any of the foregoing, wherein:
R^{a} is selected from alcohol protecting groups, and
R¹ is selected from —N(Boc)₂, —NHBoc, —N(Phth), —NH(Cbz), and —NO₂.

87. The method according to Embodiment 86, wherein R^{a} is benzyl (Bn).

88. The method according to Embodiment 86 or 87, wherein converting the compound of Formula XII, or a stereoisomer of the compound of Formula XII, or a deuterated derivative of the compound of Formula XII or a stereoisomer thereof, or salts of any of the foregoing, into the compound of Formula XI, or a stereoisomer thereof, or a deuterated derivative of the compound of Formula XI or a stereoisomer thereof, or salts of any of the foregoing, comprises a reaction that is performed in the presence of a ruthenium catalyst.

89. The method according to Embodiment 88, wherein the ruthenium catalyst is selected from dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zahn 1B catalyst); dichloro[1,3-bis(2,4,6-trimethylphenyl)-2-imidazolidinylidene](2-isopropoxyphenylmethylene)ruthenium(II); dichloro[1,3-bis(2,6-isopropylphenyl)-2-imidazolidinylidene](2-isopropoxyphenylmethylene)ruthenium(II); (1,3-dimesitylimidazolidin-2-ylidene)dichloro(2-isopropoxy-5-nitrobenzylidene)ruthenium(II); dichloro(3-phenyl-TH-inden-1-ylidene)bis(tricyclohexylphosphine)ruthenium(II); dichloro[1,3-bis(2,4,6-trimethylphenyl)-2-imidazolidinylidene](3-phenyl-TH-inden-1-ylidene)(tricyclohexylphosphine)ruthenium(II); and dichloro(2-isopropoxyphenylmethylene) (tricyclohexylphosphine)ruthenium(II).

90. The method according to Embodiment 88 or 89, wherein the ruthenium catalyst is dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zahn 1B catalyst).

91. The method according to any one of Embodiments 78 to 90, wherein the method comprises reacting Formula XIII:

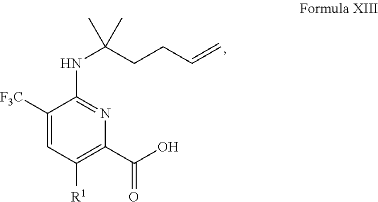

or a deuterated derivative of Compound 2, or a salt of any of the foregoing, with a compound of Formula IV:

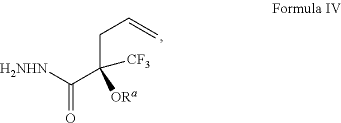

or a stereoisomer of the compound of Formula IV, or a deuterated derivative of the compound of Formula IV or a stereoisomer thereof, or salts of any of the foregoing, to produce the compound of Formula III, or a stereoisomer thereof, or a deuterated derivative of the compound of Formula III or a stereoisomer thereof, or salts of any of the foregoing, wherein:
R^{a} is selected from alcohol protecting groups, and
R¹ is selected from —N(Boc)₂, —NHBoc, —N(Phth), —NH(Cbz), and —NO₂.

92. The method according to Embodiment 91, wherein R^{a} is benzyl (Bn).

93. The method according to Embodiment 91 or 92, wherein reacting Formula XIII, or a deuterated derivative of Formula XIII, or a salt of any of the foregoing, with a compound of Formula IV, or a stereoisomer of the compound of Formula IV, or a deuterated derivative of the compound of Formula IV or a stereoisomer thereof, or salts of any of the foregoing, is performed in the presence of a peptide coupling agent and a base.

94. The method according to Embodiment 93, wherein the peptide coupling agent is selected from 2-chloro-4,6-dimethoxy-1,3,5-triazine (CDMT), 1,1-carbonyl diimidazole (CDI), 2-chloro-1-methylpyridinium iodide (CMPI), 1-[(1-(cyano-2-ethoxy-2-oxoethylideneaminooxy)dimethylamino-morpholinomethylene)]methanaminium hexafluorophosphate (COMU), isobutyl chloroformate (IBCF), and propanephosphonic acid anhydride (T3P).

95. The method according to Embodiment 93 or 94, wherein the peptide coupling agent is propanephosphonic acid anhydride (T3P).

### EXAMPLES

Table of Abbreviations

| Abbreviation | Chemical Name |
|---|---|
| AcOH | acetic acid |
| anh. | anhydrous |
| aq. | aqueous |
| AgSbF₆ | silver hexafluoroantimonate(V) |
| BnBr | benzyl bromide |
| BnOH | benzyl alcohol |
| BINAP | 2,2′-bis(diphenylphosphino)-1,1′-binapthalene |
| Boc | tert-butyloxycarbonyl |
| Boc-NHNH₂ | tert-butyl carbazate |
| Boc₂O | di-tert-butyl dicarbonate; Boc anhydride |
| BOP | benzotriazol-1-yloxytris(dimethylamino)phosphonium |
| | hexafluorophosphate |
| n-BuLi | n-butyl lithium |
| n-Bu₄NF•H₂O | tetra-n-butylammonium fluoride monohydrate |
| t-BuOH | tert-BuOH; tert-butanol |
| t-BuOK (KOtBu) | potassium tert-butoxide |
| t-BuONa | sodium tert-butoxide |
| Bz-Cl | benzoyl chloride |
| CDCl₃ | chloroform-d |
| CDI | 1,1-carbonyl diimidazole |
| CD₃OD | methyl-d₄ alcohol-d |
| CDMT | 2-chloro-4,6-dimethoxy-1,3,5-triazine |
| CF₃CO₂H | trifluoroacetic acid; perfluoroacetic acid; |
| | trifluoroethanoic acid |
| CH₃CN | acetonitrile |
| CO₂ | carbon dioxide |
| CsOAc | cesium acetate |
| Cs₂CO₃ | cesium carbonate |
| CuI | copper(I) iodide |
| DABCO | 1,4-diazabicyclo[2.2.2]octane |
| DCHA | dicyclohexylamine |
| DCE | 1,2-dichloroethane |
| DCM (CH₂Cl₂) | dichloromethane; methylene chloride |
| DI | deionized |
| DIAD | diisopropyl azodicarboxylate |
| DIEA (DIPEA) | N,N-diisopropylethylamine |
| DMAP | 4-(dimethylamino)pyridine |
| DMF | N,N-dimethylformamide |
| DMP | Dess-Martin periodinane |
| DMSO | dimethyl sulfoxide |
| DMSO-d6 | dimethyl sulfoxide-d6 |
| EDCI | 1-ethyl-3-(3-dimethylaminopropyl)carbodiimide |
| ELSD | evaporative light scattering detector |
| ESI-MS | electrospray ionization mass spectrometry |
| Et₂O | diethyl ether |
| Et₃N (TEA) | triethylamine |
| EtOAc | ethyl acetate |
| EtOH | ethanol |
| FIH | first in human |
| Fe | iron |
| ¹⁹F NMR | fluorine nuclear magnetic resonance |
| Grubbs catalyst | [1,3-bis(2,4,6-trimethylphenyl)imidazolidin-2-ylidene]- |
| 2^{nd} generation | dichloro-[(2-isopropoxyphenyl)methylene]ruthenium |
| H₂ | hydrogen gas |
| H₂O | water |
| HATU | 1-[bis(dimethylamino)methylene]-1H-1,2,3- |
| | triazolo[4,5-b]pyridinium 3-oxide hexafluorophosphate |
| HCl | hydrochloric acid |
| HCN | hydrogen cyanide |
| HPLC | high pressure liquid chromatography |
| HMPA | hexamethylphosphoramide |
| ¹H NMR | proton nuclear magnetic resonance |
| HNL | hydroxynitrile lyase enzyme |
| HNO₃ | nitric acid |
| HOAc (AcOH) | acetic acid |
| HOBT | hydroxybenzotriazole |
| HPLC | high performance liquid chromatography |
| H₂O | water |
| H₂O₂ | hydrogen peroxide |
| H₂SO₄ | sulfuric acid |
| I₂ | iodine |
| IPAc (iPrOAc) | isopropyl acetate |
| K₂CO₃ | potassium carbonate |
| KHCO₃ | potassium bicarbonate |
| KHSO₄ | potassium bisulfate |
| KMnO₄ | potassium permanganate |
| KOH | potassium hydroxide |
| K₃PO₄ | potassium phosphate tribasic |
| KRED | ketoreductase |
| LC | liquid chromatography |
| LCMS | liquid chromatography mass spectrometry |
| Li | lithium metal |
| LiAlH₄ | lithium aluminum hydride |
| LiOH | lithium hydroxide |
| MeOH | methanol |
| MeCN (CH₃CN; ACN) | acetonitrile |
| MeMgCl | methyl magnesium chloride |
| 2-MeTHF | 2-methyltetrahydrofuran |
| MgSO₄ | magnesium sulfate |
| MnO₂ | manganese dioxide |
| MS | mass spectrometry |
| MTBE | methyl tert-butyl ether |
| NH₄HCO₂ | ammonium formate |
| Na | sodium |
| NaCl | sodium chloride |
| NaH: | sodium hydride |
| NaHCO₃ | sodium bicarbonate |
| NaIO₄ | sodium periodate |
| NaSMe | sodium thiomethoxide (sodium methanethiolate) |
| NaOAc | sodium acetate |
| NaOH | sodium hydroxide |
| Na₂S₂O₄ | sodium dithionite |
| Na₂S₂O₃ | sodium thiosulfate |
| Na₂SO₄ | sodium sulfate |
| NBS | N-bromosuccinimide |
| NH₃ | ammonia |
| NH₄Cl | ammonium chloride |
| NH₄HCO₃ | ammonium bicarbonate |
| NLT | no less then |
| NMM | N-methylmorpholine, 4-methylmorpholine |
| NMP | N-methyl-2-pyrrolidone |
| NMR | nuclear magnetic resonance |
| N₂ | dinitrogen |
| MTBE | methyl tert-butyl ether |
| OsO₄ | osmium tetroxide |
| Pd/C | palladium on carbon |
| PdCl₂ | palladium(II) chloride |
| PdCl₂(S-BINAP) | dichloro[2,2′-bis(diphenylphosphino)-1,1′- |
| | binaphthyl]palladium(II) |
| Pd₂(dba)₃ | tris(dibenzylideneacetone)dipalladium(0) |
| Pd(dppf)Cl₂ | 1,1′-bis(diphenylphosphino)ferrocene palladium(II) |
| | chloride |
| Pd(OAc)₂ | palladium (II) acetate |
| PE:EA | petroleum ether:ethyl acetate |
| PhI(OAc)₂ | (diacetoxyiodo)benzene |
| PhMe | toluene |
| PPh₃ | triphenylphosphine |
| POCl₃ | phosphoryl chloride |
| PtO₂ | platinum oxide |
| QNMR | quantitative nuclear magnetic resonance |
| RBF | round bottom flask |
| RCM | ring closing metathesis |
| RT | room temperature |
| RuCl₃ | ruthenium(III) chloride |
| RuCl₂(mesitylene) | dichloro(mesitylene)ruthenium(II) |
| SFC | supercritical fluid chromatography |
| Silica Cat Pd | palladium on silica |
| SiO₂ | silicon dioxide; silica; silica gel |
| SM | starting material |
| TBAF | tetrabutylammonium fluoride |
| TBAI | tetrabutylammonium iodide |
| TBD | 1,5,7-Triazabicyclo[4.4.0]dec-5-ene |
| TBDPS | tert-butyldiphenylsilyl |
| TBDPSiCl (TBDPS-Cl | tert-butyl(chloro)diphenylsilane |
| or TBDPSCl) | |
| TEA (Et₃N) | triethylamine |
| TFA | trifluoroacetic acid |
| TFAA | trifluoroacetic anhydride |
| THF | tetrahydrofuran |
| Ti(OEt)₄ | titanium (IV)ethoxide |
| Tⱼ | temperature of jacket |
| TLC | thin layer chromatography |
| TMEDA | tetramethylethylenediamine |
| TMSCF₃ | trifluoromethyltrimethylsilane |
| Tᵣ | temperature of reactor |
| T3P (PPAA) | propanephosphonic acid anhydride |
| TPPO (Ph₃PO) | triphenylphospine oxide |
| p-TsCl; TsCl | p-toluenesulfonyl chloride; tosyl chloride |
| (S,S)-TsDPEN | (1S,2S)-N-(p-toluenesulfonyl)-1,2- |
| | diphenylethanediamine |
| UHP | urea hydrogen peroxide |
| Umicore M101 | Grubbs Catalyst ® M101 (Sigma Aldrich Cat. No. |
| Ru-Catalyst | 774901): dichloro(3-phenyl-1H-inden-1- |
| | ylidene)bis(tricyclohexylphosphine)ruthenium(II) |
| UPLC | ultra performance liquid chromatography |
| w/v | weight by volume |
| w/w | weight by weight |
| wt | weight |
| XantPhos | 4,5-bis(diphenylphosphino)-9,9-dimethylxanthene |
| XantPhos Pd G4 | (6-diphenylphosphanyl-10H-phenoxazin-4-yl)- |
| | diphenylphosphane; methanesulfonic acid; N-methyl-2- |
| | phenylaniline; palladium (Sigma Aldrich Catalog No. |
| | 900329) |
| Zhan catalyst-1B | dichloro[1,3-bis(2,4,6-trimethylphenyl)-2- |
| | imidazolidinylidene][[5-[(dimethylamino)sulfonyl]-2- |
| | (1-methylethoxy-O)phenyl]methylene-C]ruthenium(II) |

### General Solid State NMR (SSNMR) Method

A Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### General Thermogravimetric Analysis (TGA) Method

TGA was used to investigate the presence of residual solvents in the lots characterized and to identify the temperature at which decomposition of the sample occurs. Unless provided otherwise in the following Examples, TGA data were collected on a TA instrument Discovery series with TRIOS system. TGA data for neat amorphous Compound I were collected on a Mettler Toledo TGA/DSC 3+STARe System.

### General Differential Scanning Calorimetry (DSC) Method

Unless provided otherwise in the following Examples, the melting point or glass transition point of the material was measured using a Mettler Toledo TGA/DSC 3+STARe System. DSC data for Compound I methanol solvate (dry) and Compound I p-toluene sulfonic acid were collected on a TA instrument Discovery series with TRIOS system.

### Example 1: Preparation of (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I)

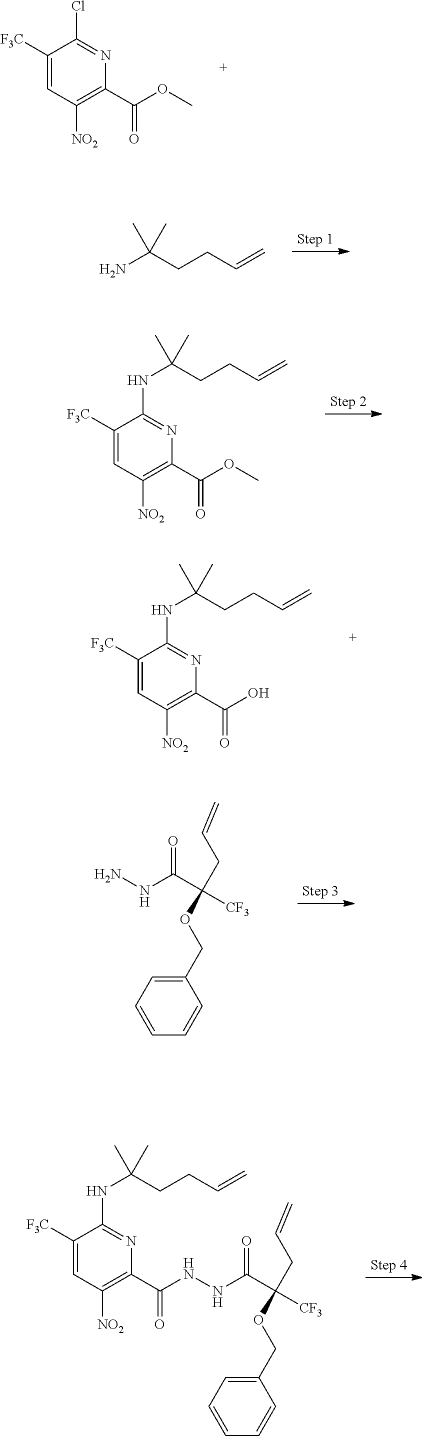

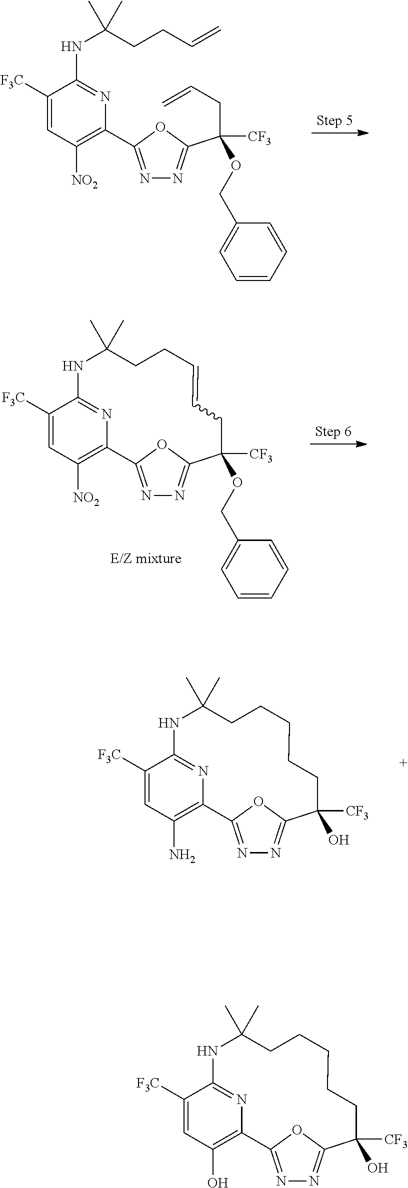 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2N)N1 ; CC1(C)CCCCC[C@](O)(C(F)(F)F)C2=NN=C(O2)c2nc(c(C(F)(F)F)cc2O)N1]

### Step 1: Methyl 6-(1,1-dimethylpent-4-enylamino)-3-nitro-5-(trifluoromethyl)pyridine-2-carboxylate

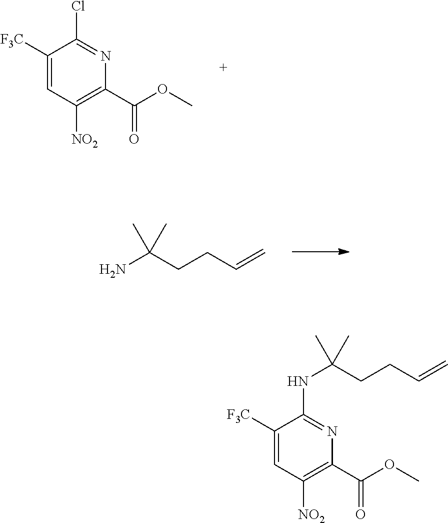

2-Methylhex-5-en-2-amine (hydrochloride salt) (69.4 g, 463.7 mmol) was suspended in acetonitrile (960 mL) and treated with DIEA (220 mL, 1.263 mol). To the formed brown solution was added methyl 6-chloro-3-nitro-5-(trifluoromethyl)pyridine-2-carboxylate (120 g, 421.7 mmol) in one portion. The orange solution was slowly heated to 65° C. over 2.5 h (Note: reaction shows exotherm on heating). The deep orange solution was evaporated at 40° C. and to the residue was added with MTBE (1 L) and water (1 L) and the layers were separated. The deep orange organic phase was washed with a 1:1 solution of saturated aqueous NH₄Cl/water mixture (2×600 mL), once with brine (400 mL) and the organic phase was dried, filtered and evaporated to give methyl 6-(1,1-dimethylpent-4-enylamino)-3-nitro-5-(trifluoromethyl) pyridine-2-carboxylate (152.7 g, 100%). ESI-MS m z calc. 361.12494, found 362.0 (M+1)⁺; Retention time: 3.02 minutes (LC Method D).

### Step 2: 6-(1,1-Dimethylpent-4-enylamino)-3-nitro-5-(trifluoromethyl)pyridine-2-carboxylic acid

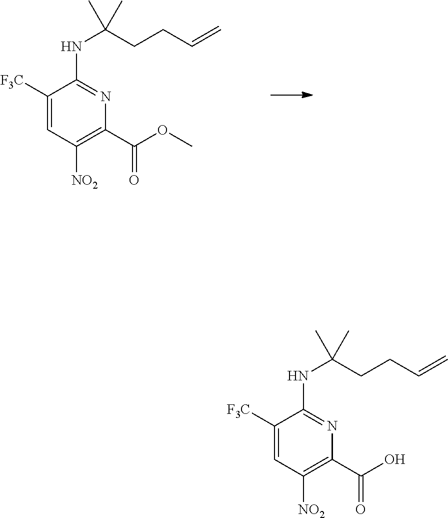 [SMILES: C=CCCC(C)(C)Nc1nc(C(=O)OC)c([N+](=O)[O-])cc1C(F)(F)F ; C=CCCC(C)(C)Nc1nc(C(=O)O)c([N+](=O)[O-])cc1C(F)(F)F]

Methyl 6-(1,1-dimethylpent-4-enylamino)-3-nitro-5-(trifluoromethyl)pyridine-2-carboxylate (152.4 g, 421.7 mmol) was dissolved in methanol (750 mL) and treated with NaOH (750 mL of 2 M, 1.500 mol) under stirring (added all at once giving a slight exotherm from 30° C. to 40° C.). The solution was stirred at room temperate for 18 h. The deep red solution was concentrated under reduced pressure at 42° C. and the resulting orange red solution was treated with toluene (1 L). The emulsion was stirred in an ice bath and acidified to pH=1 by addition of HCl (260 mL of 6 M, 1.560 mol) keeping the internal temperature below 15° C. The phases were separated and the organic phase was washed twice with water (2×500 mL) and once with brine (400 mL). The organic phase was dried over MgSO₄, filtered, evaporated and dried under vacuum to give 137 g of a deep orange mass of solid. This material was evaporated from acetonitrile (˜1 L, to remove residual toluene) and dissolved in acetonitrile (600 mL) and warmed to −60° C. To the deep red hot solution was added N-cyclohexylcyclohexanamine (79 mL, 396.5 mmol) under stirring (exotherm noted from 60° C. to 70° C.) and the hot solution was seeded. The material became a solid mass at an internal temperature of ˜60° C., which could be stirred magnetically after breaking up. The thick suspension was stirred in the cooling hot water bath overnight and then in an ice bath for 3 h. The solid was collected by filtration, washed with cold acetonitrile until the filtrate was colorless and dried over the weekend to give 6-(1,1-dimethylpent-4-enylamino)-3-nitro-5-(trifluoromethyl)pyridine-2-carboxylic acid (dicyclohexylamine salt) (172 g, 77%) as a yellow solid. This salt was suspended in MTBE (1 L) and treated with citric acid (1.2 L of 1 M, 1.200 mol). The mixture was stirred and the phases were separated. The organic phase was washed twice more with 1 M citric acid (2×400 mL) and 4 times with 0.5M KHSO₄ (4×400 mL). The organic phase was then washed once with brine (200 mL), dried, filtered and evaporated to give 6-(1,1-dimethylpent-4-enylamino)-3-nitro-5-(trifluoromethyl)pyridine-2-carboxylic acid (113.4 g, 77%) as a yellow orange oil, which crystallized upon standing. ¹H NMR (400 MHz, DMSO-d6) δ 14.21 (s, 1H), 8.46 (s, 1H), 6.20-6.00 (m, 1H), 5.82-5.57 (m, 1H), 5.13-4.74 (m, 2H), 1.97 (d, J 2.9 Hz, 4H), 1.45 (s, 6H) ppm. ESI-MS m/z calc. 347.10928, found 348.0 (M+1)⁺; Retention time: 2.49 minutes (LC Method D).

### Step 3: N′-[(2R)-2-Benzyloxy-2-(trifluoromethyl)pent-4-enoyl]-6-(1,1-dimethylpent-4-enylamino)-3-nitro-5-(trifluoromethyl)pyridine-2-carbohydrazide

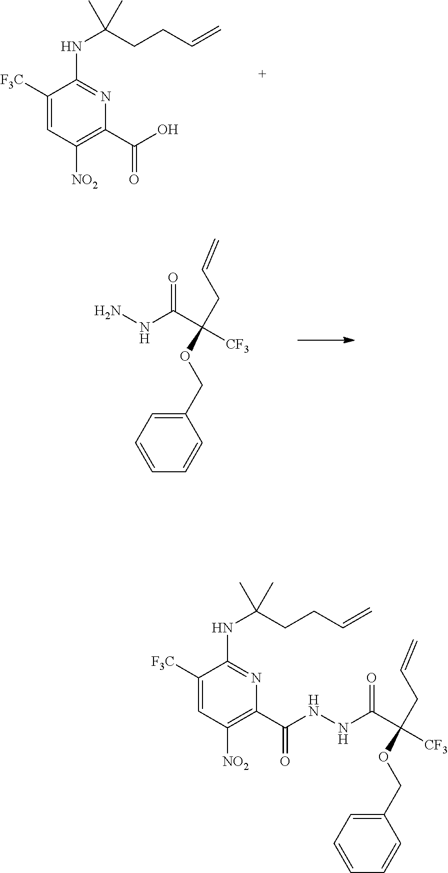 [SMILES: C=CCCC(C)(C)Nc1nc(C(=O)O)c([N+](=O)[O-])cc1C(F)(F)F ; C=CC[C@@](OCc1ccccc1)(C(=O)NN)C(F)(F)F ; C=CCCC(C)(C)Nc1nc(C(=O)NNC(=O)[C@@](CC=C)(OCc2ccccc2)C(F)(F)F)c([N+](=O)[O-])cc1C(F)(F)F]

6-(1,1-Dimethylpent-4-enylamino)-3-nitro-5-(trifluoromethyl)pyridine-2-carboxylic acid (100 g, 285.1 mmol) and (2R)-2-benzyloxy-2-(trifluoromethyl)pent-4-enehydrazide (86.3 g, 299.4 mmol) were dissolved in DMF (600 mL) and cooled in an ice bath. At an internal temperature of 3.1° C., HATU (114 g, 299.8 mmol) was added in one portion (no exotherm observed). Then, DIEA (100 mL, 574.1 mmol) was slowly added over 0.5 h (exothermic) keeping the internal temperate between 3 and 10° C. After the addition, the ice bath was removed and the reaction was stirred for another 0.5 h allowing it to warm to room temperature. The orange solution was added to a stirred solution of ice and water (3 L) and MTBE (1 L). The mixture was stirred for 10 minutes and the phases were separated. The organic phase was washed twice with water (2×1 L), 0.2 M KHSO₄ (3×1 L) and once with brine (250 mL). The organic phase was dried, filtered and evaporated to give as an orange mass, N′-[(2R)-2-benzyloxy-2-(trifluoromethyl)pent-4-enoyl]-6-(1,1-dimethylpent-4-enylamino)-3-nitro-5-(trifluoromethyl) pyridine-2-carbohydrazide (181 g, quantitative yield). ESI-MS m/z calc. 617.2073, found 618.0 (M+1)⁺; Retention time: 3.25 minutes (LC Method D). This material was used directly in the next step.

### Step 4: 6-[5-[(1R)-1-Benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine

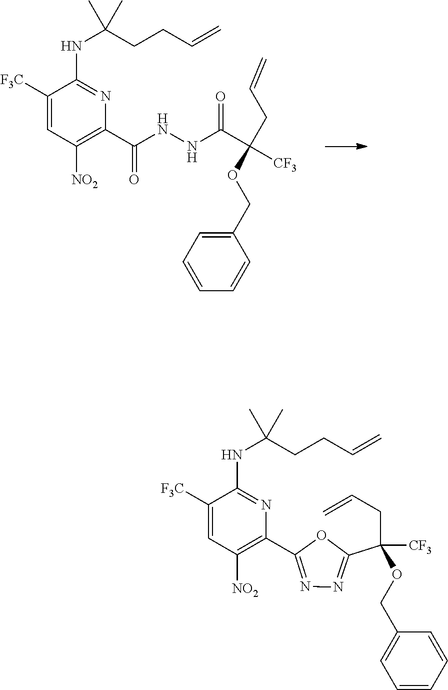 [SMILES: C=CCCC(C)(C)Nc1nc(C(=O)NNC(=O)[C@@](CC=C)(OCc2ccccc2)C(F)(F)F)c([N+](=O)[O-])cc1C(F)(F)F ; C=CCCC(C)(C)Nc1nc(-c2nnc([C@@](CC=C)(OCc3ccccc3)C(F)(F)F)o2)c([N+](=O)[O-])cc1C(F)(F)F]

N′-[(2R)-2-Benzyloxy-2-(trifluoromethyl)pent-4-enoyl]-6-(1,1-dimethylpent-4-enylamino)-3-nitro-5-(trifluoromethyl)pyridine-2-carbohydrazide (176.1 g, 285.2 mmol) was dissolved in acetonitrile (1.4 L) and heated to 55° C. The yellow orange solution was treated with DIEA (124 mL, 711.9 mmol) followed by portion-wise addition of tosyl chloride (54.4 g, 285.3 mmol) over 15 min (exothermic, internal temperature kept between 55° C. and 60° C. by removing the heating mantel and slow addition) and the reaction was stirred at 55-60° C. for 45 min. The reaction solution was concentrated under reduced pressure at 40° C. and the residue was extracted with MTBE/heptane 1:1 (1.4 L) and water (1.4 L). The organic phase was washed once more with water (1.5 L), twice with 0.2M KHSO₄ (2×1 L) and once with brine (0.5 L). The organic phase was dried, filtered and evaporated to give 172 g of an orange oil which was dissolved in 100 mL of toluene and 300 mL of heptane. The solution was loaded onto a 3 kg silica column (column volume=4800 mL, flow rate=900 mL/min). Eluted with 100% hexanes for 1 min, then programmed an initial gradient of 0% to 10% ethyl acetate in hexanes over 106 min (2 column volumes). The product started eluting at −4% ethyl acetate, so 4.3% ethyl acetate was held isocratically until the product finished eluting to give 6-[5-[(1R)-1-benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine (139.1 g, 80%). ¹H NMR (400 MHz, Chloroform-d) δ 8.51 (s, 1H), 7.40-7.27 (m, 5H), 6.03-5.87 (m, 1H), 5.80-5.66 (m, 1H), 5.58 (s, 1H), 5.31-5.16 (m, 2H), 5.03-4.95 (m, 1H), 4.95-4.89 (m, 1H), 4.81 (d, J 10.5 Hz, 1H), 4.64 (d, J 10.5 Hz, 1H), 3.28-3.13 (m, 2H), 2.08-1.99 (m, 2H), 1.99-1.89 (m, 2H), 1.47 (s, 6H) ppm. ESI-MS m/z calc. 599.1967, found 600.0 (M+1)⁺; Retention time: 3.71 minutes (LC Method D).

### Step 5: (6R)-6-Benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture)

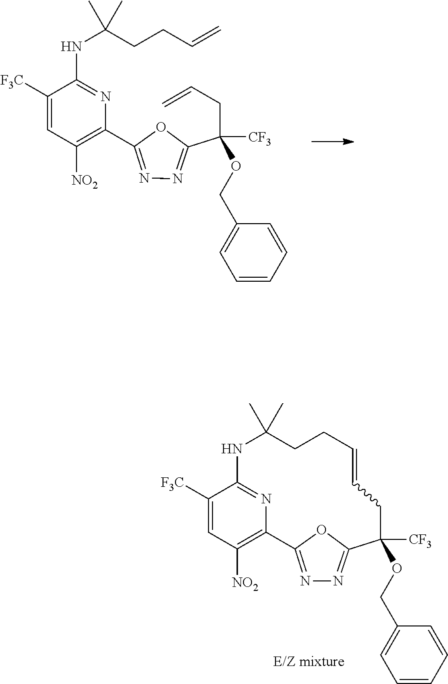 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1]

This reaction was run in three, 46.3 g batches in parallel, each in a 12 L, 3-neck round bottom flask. The experimental below describes one of these batches.

Attached a sparging tube, reflux condenser with gas bubbler & overhead stirrer to a 12 L vessel placed in heat blanket. Dissolved 6-[5-[(1R)-1-benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine (46.3 g, 76.22 mmol) in DCE (8.23 L). Sparged the system with a heavy stream of nitrogen gas. Set the heat blanket for 50° C. When the vessel reached 53° C., added dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zhan catalyst-1B, 11.2 g, 15.26 mmol) all at once. Rinsed the catalyst container with DCE and added the rinse to the reaction. On completion of catalyst addition, increased the blanket temperature to 73° C. Once internal temperature reached 72° C., continued stirring for 2 h 28 min then decreased the heat blanket temperature to 45° C. After 2 h 27 min, internal temperature reached 50° C. After 15 min, added solid 2-sulfanylpyridine-3-carboxylic acid (12 g, 77.33 mmol) and triethylamine (11 mL, 78.92 mmol). Stirred for 12 h then allowed the mixture to cool to room temperature. Added 100 g of SiO₂ and 10 g of activated carbon (20-40 mesh, granular) to the reaction. Stirred for 1 h then filtered over Celite and evaporated the filtrate giving the crude product mixture. Combined the material from all three parallel reactions to give 71.2 g of crude product mixture. This material was purified on two separate 3 kg silica gel columns using a gradient from 100% hexanes to 10% ethyl acetate in hexanes over 110 min followed by a gradient from 10% ethyl acetate in hexanes to 100% ethyl acetate over 10 minutes. After combining the two separate purified batches, obtained (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture) (51.88 g, 40%). ¹H NMR (400 MHz, DMSO-d6) δ 8.55 (d, J 0.8 Hz, 1H), 7.49-7.21 (m, 5H), 6.58 (s, 1H), 5.79 (dt, J 13.7, 6.5 Hz, 1H), 5.58 (ddd, J 15.0, 8.8, 5.6 Hz, 1H), 4.83 (d, J 11.1 Hz, 1H), 4.55 (d, J 11.1 Hz, 1H), 3.13 (dd, J 14.2, 5.4 Hz, 1H), 2.77 (dd, J 14.3, 8.8 Hz, 1H), 2.38-2.24 (m, 1H), 2.14-1.93 (m, 3H), 1.58-1.32 (m, 6H) ppm. ESI-MS m/z calc. 571.1654, found 572.1 (M+1)⁺; Retention times: 3.46 minutes and 3.49 minutes (LC Method D). Product formed as a 3:1 mixture of double bond isomers.

### Step 6: (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I) and its enantiomer

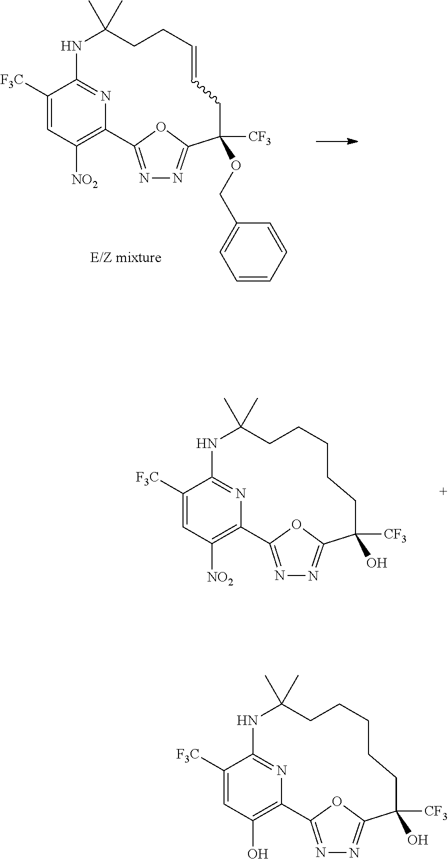 [SMILES: CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2O)N1]

(6R)-6-Benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture) (50.8 g, 88.89 mmol) was dissolved in 250 mL of ethanol and partially concentrated by rotary evaporation with 28° C. water bath to remove any residual solvents then dissolved in further ethanol (720 mL) in a 5 L flask. Degassed the solution using 5 cycles of house vacuum with nitrogen gas backfill. Added dihydroxypalladium (15.2 g of 10% w/w, 10.824 mmol) to the substrate solution under nitrogen. Repeated house vacuum with hydrogen backfill for 6 cycles to replace nitrogen atmosphere with hydrogen. Finally kept the vessel under 1 atmosphere of hydrogen using a balloon. Stirred this mixture vigorously with a magnetic stirrer overnight then removed the hydrogen balloon. Filtered the mixture through 70 g of Celite on a medium-fritted funnel. Concentrated the green filtrate solution by rotary evaporation with a 28° C. water bath. Obtained 42.65 g of crude product as a yellow solid of which 41.5 g was purified by reverse-phase chromatography (dissolved in 125 mL of methanol and 2.55 mL DMF (2% DMF/methanol solution) and loaded onto a 3.8 kg C₁₈ column (column volume=3.3 L, flow rate=375 mL/min). Programmed an initial gradient of 40% to 70% acetonitrile in water over 176 minutes (20 column volumes), then brought the eluent to 100% acetonitrile over the following ˜20 min). Mixed and pure fractions were isolated from the column. Pure fractions were concentrated to give as a yellow solid, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (28.17 g, 70%). This material was combined with several smaller batches (80 mg, 340 mg, 360 mg, 1.46 g and 1.63 g) made by similar methods as a solution in acetonitrile which was then concentrated to give a yellow solid. This solid was dissolved in dichloromethane and heptane was added then the solution was concentrated under vacuum in the dark at 40° C. overnight, which gave 31.95 g of (6R)-17-amino-12,12-dimethyl-6,15-bis(trifluoromethyl)-19-oxa-3,4,13,18-tetrazatricyclo [12.3.1.12,5]nonadeca-1(18),2,4,14,16-pentaen-6-ol. ¹H NMR (400 MHz, DMSO-d6) δ 7.61 (s, 1H), 7.59 (s, 1H), 5.96 (s, 2H), 4.64 (s, 1H), 2.90-2.71 (m, 1H), 2.30-2.15 (m, 1H), 2.15-1.98 (m, 1H), 1.91-1.74 (m, 1H), 1.73-1.57 (m, 1H), 1.56-1.38 (m, 5H), 1.36 (s, 3H), 1.31 (s, 3H). ESI-MS m/z calc. 453.15994, found 454.2 (M+1)⁺; Retention time: 3.03 minutes (LC Method D).

One mixed fraction from the reverse-phase purification described above contained an impurity showing a mass one unit greater than the intended product described above. This fraction was concentrated and the residue was dissolved in 3.6 mL of methanol, then purified by reverse-phase prep HPLC through a C₁₈ column using a gradient from 1-99% acetonitrile in water (+HCl modifier) giving as a yellow solid, (6R)-12,12-dimethyl-6,15-bis(trifluoromethyl)-19-oxa-3,4,13,18-tetrazatricyclo[12.3.1.12,5]nonadeca-1(18),2,4,14,16-pentaene-6,17-diol (105 mg, 0.003%). ¹H NMR (400 MHz, DMSO-d6) δ 10.43 (s, 1H), 7.64 (s, 1H), 7.57 (s, 1H), 4.85 (s, 1H), 2.91-2.74 (m, 1H), 2.30-2.15 (m, 1H), 2.10-1.96 (m, 1H), 1.85-1.68 (m, 1H), 1.68-1.54 (m, 1H), 1.53-1.37 (m, 5H), 1.36 (s, 3H), 1.31 (s, 3H) ppm. ESI-MS m/z calc. 454.14395, found 455.2 (M+1); Retention time: 2.87 minutes (LC Method D).

### Step 7: Solid form characterization of crystalline Compound I Form A (neat)

### A. X-Ray Powder Diffraction

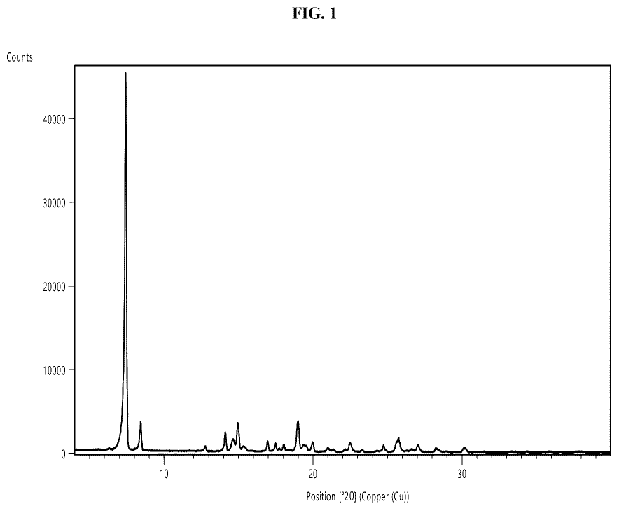

The XRPD diffractogram for crystalline Compound I Form A (neat) produced by Step 6 and recrystallized from EtOH was acquired using the General X-Ray Powder Diffraction (XRPD) Method. The XRPD diffractogram for crystalline Compound I Form A (neat) is provided in FIG. 1, and the XRPD data are summarized below.

XRPD signals for crystalline Compound I Form A (neat)

| XRPD | Angle (degrees | Intensity |
|---|---|---|
| Signal No. | 2-Theta ± 0.2) | % |
| 1 | 7.4271 | 100 |
| 2 | 8.4377 | 7.56 |
| 3 | 14.1039 | 4.84 |
| 4 | 14.5744 | 2.53 |
| 5 | 14.962 | 7.2 |
| 6 | 16.9424 | 2.49 |
| 7 | 19.0503 | 5.71 |
| 8 | 19.9711 | 2.4 |
| 9 | 22.4778 | 2.31 |
| 10 | 25.5622 | 2.19 |
| 11 | 25.7502 | 3.64 |

### B. Differential Scanning Calorimetry Analysis

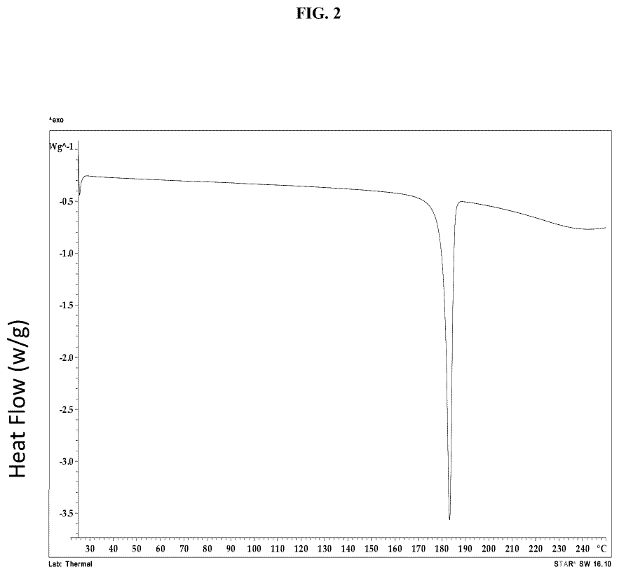

The DSC data were collected with a ramp of 10.00° C./min to 250.00° C. The DSC thermogram for crystalline Compound I Form A (neat) produced by Step 6 is provided in FIG. 2. The thermogram shows a Tm onset of 180.8° C., with a Tm peak at 183.18° C., 62.32 J/g.

### Example 2: Bioactivity of Compound I

Ussing Chamber Assay of CFTR-mediated short-circuit currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101 and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### Identification of Potentiator Compounds

The activity of the CFTR potentiator compounds on the CFTR-mediated I_{SC} was determined in Ussing chamber studies as described above. The F508del/MF-HBE cell cultures were incubated with the potentiator compounds at a range of concentrations in combination with 10 μM (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione for 18-24 hours at 37° C. and in the presence of 20% human serum. The concentration of potentiator compounds and (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo [17.3.1.111,14.05,10]tetracosa-1(22),5,7,9,19(23),20-hexaene-2,2,4-trione used during the 18-24 hours incubations was kept constant throughout the Ussing chamber measurement of the CFTR-mediated I_{SC} to ensure compounds were present throughout the entire experiment. The efficacy and potency of the putative F508del potentiators was compared to that of the known Vertex potentiator, ivacaftor (N-[2,4-bis(1,1-dimethylethyl)-5-hydroxyphenyl]-1,4-dihydro-4-oxoquinoline-3-carboxamide).

The CFTR-modulating activity for Compound I using the assay described in this example had an EC₅₀ of <500 nM.

### Example 3: Crystalline forms of Compound I

1. Compound I Methanol Solvate (wet)

### A. Synthetic Procedure

A 30 mL vial with a magnetic stir bar was charged with Compound I neat Form A (0.15 g), water (1.1 mL) and Methanol (3.3 mL). The sealed vial was heated to 65° C. When the slurry solution turned homogeneous the heating block was turned off and the stirring was stopped. The solution was allowed to cool naturally without stirring. The solution self-nucleated within an hour and the unstirred solution continued to cool and was allowed to sit at room temperature over 3 days.

### B. X-Ray Powder Diffraction

The powder, X-ray powder diffraction (XRPD), diffractogram of Compound I methanol solvate (wet) was acquired at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40°2θ with a step size of 0.0131303° and 49 s per step. The results are shown in FIG. 3 and summarized in the table below:

XRPD Results for Compound I Methanol Solvate (wet)

| XRPD | Angle (Degrees | Intensity |
|---|---|---|
| Signals | 2-Theta ± 0.2) | % |
| 1 | 25.5 | 100.0 |
| 2 | 8.4 | 66.6 |
| 3 | 20.5 | 61.4 |
| 4 | 16.9 | 51.8 |
| 5 | 14.6 | 24.9 |
| 6 | 18.6 | 21.1 |
| 7 | 19.0 | 15.7 |
| 8 | 21.0 | 12.7 |
| 9 | 15.0 | 12.5 |
| 10 | 18.9 | 11.7 |

### C. Single Crystal Analysis

Single crystals having the Compound I methanol solvate (wet) structure were obtained. X-ray diffraction data were acquired at 100K on a Rigaku diffractometer equipped with Cu K_{α} radiation (λ=1.54178 Å) and a CMOS detector. The structure was solved and refined using SHELX programs (Sheldrick, G.M., Acta Cryst., (2008) A64, 112-122) and results are summarized below.

### Single Crystal Elucidation of Compound I Methanol Solvate (Wet)

| | |
|---|---|
| Crystal System | Monoclinic |
| Space Group | P2₁ |
| a (Å) | 6.67183(3) |
| b (Å) | 41.5377(3) |
| c (Å) | 14.18151(7) |
| α (°) | 90 |
| β (°) | 92.0858(4) |
| γ (°) | 90 |
| V (Å³) | 3980.53(4) |
| Z/Z′ | 2/4 |
| Temperature | 100 K |

### D. Solid State NMR

The results of ¹³C CPMAS SSNMR are shown in FIG. 4 and ¹⁹F MAS SSNMR are shown in FIG. 5 and summarized below.

¹³C CPMAS SSNMR Results for Compound I Methanol Solvate (wet)

| | Chem Shift | Intensity |
|---|---|---|
| Signal | [ppm] | [rel] |
| 1 | 164.2 | 58.6 |
| 2 | 162.8 | 59.5 |
| 3 | 145.6 | 71.6 |
| 4 | 132.5 | 79.3 |
| 5 | 124.5 | 48.4 |
| 6 | 122.1 | 28.1 |
| 7 | 120.6 | 56.6 |
| 8 | 113.1 | 78.9 |
| 9 | 73.5 | 91.8 |
| 10 | 55.9 | 100.0 |
| 11 | 49.7 | 19.2 |
| 12 | 35.2 | 66.1 |
| 13 | 31.1 | 63.6 |
| 14 | 30.5 | 68.7 |
| 15 | 29.3 | 57.5 |
| 16 | 27.4 | 56.4 |
| 17 | 24.8 | 85.6 |
| 18 | 21.0 | 50.4 |
| 19 | 19.0 | 62.1 |

¹⁹F MAS SSNMR Results for Compound I Methanol Solvate (wet)

| | Chem Shift | Intensity |
|---|---|---|
| Signal | [ppm] | [rel] |
| 1 | −63.9 | 12.5 |
| 2 | −76.6 | 1.6 |
| 3 | −79.7 | 2.9 |

2. Compound I Methanol Solvate (dry)

### A. Synthetic Procedure

A 30 mL vial with a magnetic stir bar was charged with Compound I neat Form A (0.15 g), water (1.1 mL) and Methanol (3.3 mL). The sealed vial was heated to 65° C. When the slurry solution turned homogeneous the heating block was turned off and the stirring was stopped. The solution was allowed to cool naturally without stirring. The solution self-nucleated within an hour and the unstirred solution continued to cool and was allowed to sit at room temperature over 3 days. Then the sample was placed in a vac dry oven at 60° C. overnight.

### B. X-Ray Powder Diffraction

The powder, X-ray powder diffraction (XRPD), diffractogram of Compound I methanol solvate (dry) was acquired at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49 s per step. The results are shown in FIG. 6 and summarized in the table below.

XRPD Results for Compound I Methanol Solvate (dry)

| XRPD | Angle (Degrees | Intensity |
|---|---|---|
| Signals | 2-Theta ± 0.2) | % |
| 1 | 19.3 | 100.0 |
| 2 | 25.9 | 79.4 |
| 3 | 15.4 | 64.9 |
| 4 | 18.1 | 48.7 |
| 5 | 7.4 | 32.7 |
| 6 | 21.4 | 24.1 |
| 7 | 14.2 | 19.7 |
| 8 | 27.2 | 18.9 |
| 9 | 26.4 | 13.9 |

### C. Thermogravimetric Analysis

Thermal gravimetric analysis of Compound I methanol solvate (dry) was measured using TA5500 Discovery TGA. The TGA thermogram (FIG. 7) shows ˜1.2% weight loss from ambient temperature up until −182° C.

### D. Differential Scanning Calorimetry Analysis

The melting point of Compound I methanol solvate (dry) was measured using the TA Instruments Q2000 DSC. The thermogram (FIG. 8) shows endotherms at −175° C. and at −186° C.

3. Compound I p-Toluene Sulfonic Acid

### A. Synthetic Procedure

Compound I neat Form A (˜45.3 mg) and −17.2 mg of p-toluene sulfonic acid were weighed into a precellys ball milling (2 mL) tube. A methanol:water mixture (60:40 v/v) (10 μl) was added into the tube. The mixture was ball milled at 7500 rpm over 3 cycles (60 s with 10 s pause). Then the material was vacuum dried at 40° C. in a vacuum dry oven.

### B. X-Ray Powder Diffraction

The powder, X-ray powder diffraction (XRPD), diffractogram of Compound I p-toluene sulfonic acid was acquired at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.01313030 and 49 s per step. The results are shown in FIG. 9 and summarized in the table below.

XRPD Results for Compound I p-Toluene Sulfonic Acid

| XRPD | Angle (Degrees | Intensity |
|---|---|---|
| Signals | 2-Theta ± 0.2) | % |
| 1 | 5.7 | 100.0 |
| 2 | 5.8 | 61.0 |
| 3 | 7.4 | 30.7 |
| 4 | 10.1 | 29.8 |
| 5 | 11.5 | 19.5 |
| 6 | 11.9 | 17.3 |
| 7 | 15.9 | 17.1 |
| 8 | 23.2 | 14.0 |
| 9 | 14.9 | 13.5 |
| 10 | 20.4 | 13.3 |
| 11 | 16.2 | 12.6 |
| 12 | 21.6 | 12.1 |
| 13 | 22.8 | 11.5 |
| 14 | 21.0 | 11.2 |
| 15 | 18.3 | 10.4 |

### C. Differential Scanning Calorimetry Analysis

The melting point of Compound I p-toluene sulfonic acid was measured using the TA Instruments Q2000 DSC. The thermogram (FIG. 10) shows endotherms at −175° C. and at −186° C.

### D. Solid State NMR

The results of ¹³C CPMAS SSNMR are shown in FIG. 11 and ¹⁹F MAS SSNMR are shown in FIG. 12 for Compound I p-toluene sulfonic acid and summarized below.

¹³C CPMAS Results for Compound I p-Toulene Sulfonic Acid

| | Chem Shift | Intensity |
|---|---|---|
| Signal | [ppm] | [rel] |
| 1 | 163.8 | 30.2 |
| 2 | 161.8 | 6.4 |
| 3 | 160.9 | 5.9 |
| 4 | 152.9 | 30.3 |
| 5 | 146.1 | 4.8 |
| 6 | 141.0 | 100.0 |
| 7 | 139.3 | 27.8 |
| 8 | 138.0 | 13.8 |
| 9 | 136.7 | 26.0 |
| 10 | 132.5 | 20.0 |
| 11 | 130.6 | 21.0 |
| 12 | 127.8 | 45.9 |
| 13 | 126.7 | 51.7 |
| 14 | 124.7 | 40.6 |
| 15 | 122.1 | 18.8 |
| 16 | 114.5 | 26.3 |
| 17 | 113.1 | 21.9 |
| 18 | 110.3 | 25.9 |
| 19 | 75.5 | 49.7 |
| 20 | 57.0 | 55.3 |
| 21 | 48.6 | 9.1 |
| 22 | 35.0 | 20.9 |
| 23 | 32.5 | 34.5 |
| 24 | 31.0 | 52.0 |
| 25 | 29.8 | 43.1 |
| 26 | 28.3 | 38.5 |
| 27 | 26.2 | 37.0 |
| 28 | 25.0 | 55.2 |
| 29 | 23.0 | 60.5 |
| 30 | 19.5 | 71.2 |

¹⁹F MAS Results for Compound I p-Toluene Sulfonic Acid

| | Chem Shift | Intensity |
|---|---|---|
| Signal | [ppm] | [rel] |
| 1 | −62.5 | 4.5 |
| 2 | −64.2 | 9.1 |
| 3 | −64.6 | 12.5 |
| 4 | −65.4 | 6.4 |
| 5 | −66.1 | 5.9 |
| 6 | −77.4 | 3.5 |
| 7 | −78.1 | 5.2 |
| 8 | −79.7 | 9.5 |
| 9 | −80.1 | 8.4 |

### 4. Neat Amorphous Compound I

### A. Synthetic Procedure

Neat amorphous material for Compound I was made using using a Mettler Toledo TGA/DSC 3+STARe System. About 8 mg of Compound I neat Form A was weighed on the DSC pan in triplicate. The temperature was increased up to 200° C. with a rate of 10° C. per minute and then cooled down to 10° C. The amorphous material was recovered from the DSC pan.

### B. X-Ray Powder Diffraction

The XRPD pattern was recorded at room temperature in continuous mode using a PANalytical Empyrean X-ray Diffract meter (Almelo, The Netherlands). The X-Ray was generated using Cu tube operated at 45 kV and 40 mA. Pixel l d detector was used with anti-scatter slit P8. The Divergence optics is Bragg Brentano High Definition (BBHD) with a 10 mm mask, ⅛ divergence slit, and ½ anti-scatter slit. The continuous scan mode utilized a 0.0131 degree step size and count time of 13.77 seconds per step, integrated over the range from 4 to 40 degrees two-theta. The powder sample was placed on an indented area within a zero background holder and flattened with a glass slide. The results are shown in FIG. 13.

### C. Thermogravimetric Analysis

TGA data for neat amorphous Compound I was collected on a Mettler Toledo TGA/DSC 3+STARe System. The thermogram (FIG. 14) showed negligible weight loss from ambient temperature up until thermal degradation.

### D. Differential Scanning Calorimetry Analysis

The glass transition point of neat amorphous Compound I was measured using DSC heat/cool/reheat method. Neat amorphous material for Compound I was made using a Mettler Toledo TGA/DSC 3+STARe System. About 3.5 mg of Compound I Form A was weighed on the DSC pan. The temperature was increased up to 200° C. with a rate of 10° C. per minute and then cooled down to −20° C. to generate a neat amorphous material. Then it was reheated up to 200° C. to detect the glass transition point. The glass transition of neat amorphous Compound I was observed at 64.8° C. and then recrystallization occurred at 110.2° C. resulted in melting at 181.1° C. The DSC thermogram is shown at FIG. 15.

### Example 4: Synthesis of Bis-amide Precursors

### Intermediate 1: Preparation of 2-Methylhex-5-en-2-amine Hydrochloride (Compound 3-HCl)

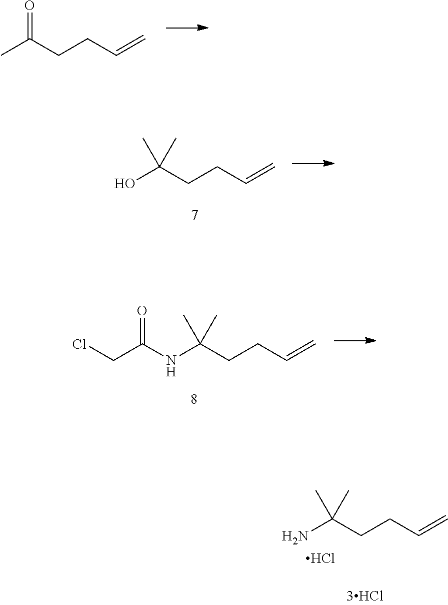

### Step 1: 2-Methylhex-5-en-2-ol (Compound 7)

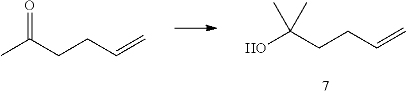

A reactor was charged with 3.0 M MeMgBr in Et₂O (1.822 kg, 5.094 L, 15.28 mol, 1.0 equiv.) and Et₂O (9 L). The mixture was cooled to 0° C. with an ice/salt bath. To the reaction mixture was added dropwise hex-5-en-2-one (CAS #109-49-9, 1,500 g, 1.77 L, 15.28 mol). The addition time was about 4.5 h. After the addition was complete the ice/salt bath was removed, and the mixture was stirred for 15 minutes at −0° C. The reaction mixture was stirred overnight, allowing it to warm up to room temperature. The reaction mixture was quenched with sat. aq. NH₄Cl (5 L) while cooling with an ice/water bath. The first 2-2.5 L added was exothermic. When 2 L of sat. aq. NH₄Cl was added the mixture, it coagulated into an unstirrable mass that became stirrable again upon further addition. To the mixture, MTBE (3 L) was added. The organic phase was separated, and the aqueous phase (still turbid with some solids) was diluted with water (3 L) and sat. aq. NH₄Cl and mixed with MTBE (5 L) to give an emulsion that was filtered over diatomaceous earth. The filter cake was rinsed with MTBE (5 L). The now clear phases were separated, and the aqueous phase was extracted once more with MTBE (3 L). The combined organic phase was washed with brine (5 L), dried over Na₂SO₄, and filtered. Removal of the solvent under reduced pressure (water bath at 55° C., down to 20 mbar) yielded a yellow oil, 1,631 g (14.28 mol, 93.5%).

### Step 2: 2-Chloro-N-(2-methylhex-5-en-2-yl)acetamide (Compound 8)

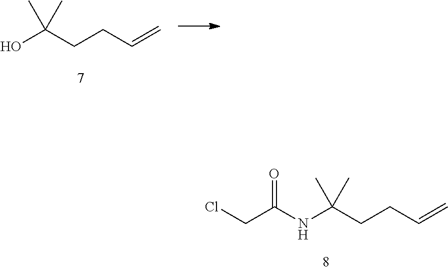

A reactor was charged with 2-methylhex-5-en-2-ol (4,822.8 g, 42.235 mol) and 2-chloroacetonitrile (17,995 g, 238.3 mol, 5.64 equiv.). The mixture was cooled to 2-0° C. (thermostat was at 0° C.) and sulfuric acid (4,305 g, 42.23 mol, 1 equiv.) was added in a small stream. Then the mixture started to warm, and the internal temperature rose to 58.4° C. (the thermostat was set at −5° C.). When the internal temperature dropped to 38° C., the addition of sulfuric acid was continued (the thermostat was set at 20° C.). Total addition took 1 h 45 m. The now dark-brown mixture was stirred for an additional hour at 24-28° C. The mixture was cooled to 15-17° C. and 34.5 L of water, followed by 30% aq. NaOH (˜8.5 L), were added until pH 10.2, keeping the internal temperature below 28° C. The mixture became beige. The mixture was added to MTBE (40 L) while stirring. Slow phase separation (˜2 h). The phases were separated, and the aqueous phase was extracted with MTBE (20 L). The combined organics were washed with water (5 L), but phase separation was very slow, so 5 L of brine was added, resulting in fast separation. The aqueous phase had pH 7-8. The organic phase was washed with 5 L of brine and dried over Na₂SO₄. After filtration (the combined Na₂SO₄ cakes were washed with 3×3 L of MTBE), the solvents were removed under reduced pressure in the reactor (the thermostat was set at 60° C., pressure to 180 mbar). About 65 L of solvent was removed. Then the heating was set at 70° C. and the less volatile liquid was distilled (down to 15 mbar). When distillation ceased, the dark-red residue was co-evaporated with 2×10.8 L of toluene. The first half of the second batch of toluene collected by distillation was sampled for ¹H-NMR, which showed about 1.2% w/w of 2-chloroacetonitrile present. The second half of the second batch of toluene collected by distillation was sampled for ¹H-NMR, which showed about 0.3% w/w of 2-chloroacetonitrile was present. Distillation was continued for 1 h more at 70° C. and 15 mbar, and some crystals appeared on the interior of the glass surface. The reactor was cooled to RT under nitrogen. The product (7,834 g) was collected by filtration.

### Step 3: 2-Methylhex-5-en-2-amine Hydrochloride (Compound 3-HCl)

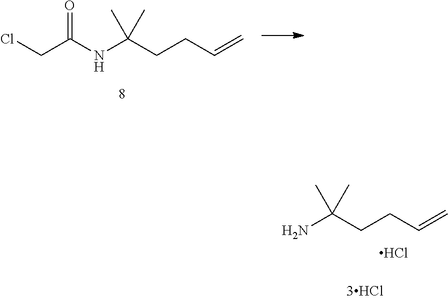

A reactor was charged with 2-chloro-N-(2-methylhex-5-en-2-yl)acetamide (3,910 g, taken as 96.8% pure, 3,785 g, 19.95 mol) and ethanol (43 L). To the stirred red-brown solution, thiourea (1,825.5 g, 23.98 mol, 1.202 equiv.) was added. The red-brown solution was heated to reflux (thermostat was set at 100° C.). After 2 h of reflux, IPC-1 showed the starting material was almost completely consumed. After 2 h 45 m of reflux, IPC-2 showed complete conversion. The mixture was cooled to 50° C. (by setting the thermostat at 50° C. and forced reflux under reduced pressure, which took 15 min.). To the red-brown solution, acetic acid (3,324 g, 55.35 mol, 2.77 equiv.) was added over the course of 10-15 min, and no temperature change was observed. The mixture was refluxed overnight (thermostat was set at 100° C.). Reflux was reached after 30-40 min, reflux temp=82.1° C. After another 30 min, a white solid precipitated. The mixture was refluxed overnight. An orange suspension was obtained. The mixture was cooled to 15-20° C. (by setting the thermostat at 15° C. and forced reflux under reduced pressure, which took 30 min to reach 28° C.). The suspension was filtered, and the filter cake was rinsed with EtOH (5×1 L). The combined filtrate (˜57 L) was concentrated under reduced pressure. A red-brown solid mass was obtained, yield 4,005 g as crude acetic acid salt.

The solid mass was dissolved in hot water (9 L) and loaded into a reactor; the flask was rinsed with another 1 L of water. DCM (22 L) was added and portion-wise Na₂CO₃ (3,355 g) was added. Up to 1.7-1.8 Kg added, the mixture stayed clear, further addition resulted in a beige emulsion. The emulsion was diluted with DCM (22 L), stirred for 30 min and allowed to settle overnight. The mixture was filtered over a pad of diatomaceous earth (˜5 cm thickness) topped with a layer of sand (˜5 cm thickness). The liquid was siphoned from the bottom of the reactor, it appeared that the organic phase was already quite well separated but a thick layer of emulsion was on the top which contained still some organic phase. The wet filter cake was rinsed with 3 L of DCM, and the filtrate from this was used to extract the aqueous phase. The combined organic phase (˜49-50 L) was dried over Na₂SO₄. The mixture was filtered, and the removed solids washed with 5 L DCM. The ˜50 L product solution was loaded into a reactor (set at −5° C. jacket temperature). To the product solution, 6 M HCl in isopropanol (5 L, 30 mol, 1.5 equiv.) was added dropwise (internal temperature at the start of addition: 2° C.). After 70 minutes the addition was complete, the internal temperature was kept <6° C. After stirring overnight at 10° C., the solvent was evaporated using a rotavap (water bath at 50° C.). The salt started to crash out of solution at 240 mbar. At 50 mbar evaporation was stopped and the contents of the flask were mixed with MTBE (22 L) and transferred to the reactor. Stirred for 18 h. The product was collected by filtration and washed with MTBE (2×2 L) to give a cream-colored solid. The solid was dried for 18 h under reduced pressure (17 mbar) at room temperature. Yield: 1,833 g (12.25 mol, 61.4%). ¹H NMR (500 MHz, DMSO-d6) δ 8.08 (s, 3H), 5.92-5.64 (m, 1H), 5.15-4.87 (m, 2H), 2.21-1.96 (m, 2H), 1.72-1.49 (m, 2H), 1.23 (s, 6H) ppm. ESI-MS m/z calc. 113.1204, found 114.5 (M+1)+.

### Intermediate 1: Alternative Preparation of 2-Methylhex-5-en-2-amine Hydrochloride (Compound 3-HCl)

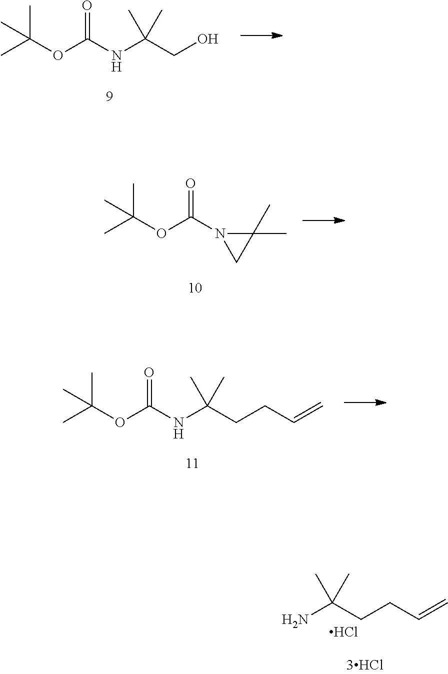

### Step 1: tert-Butyl 2,2-dimethylaziridine-1-carboxylate (Compound 10)

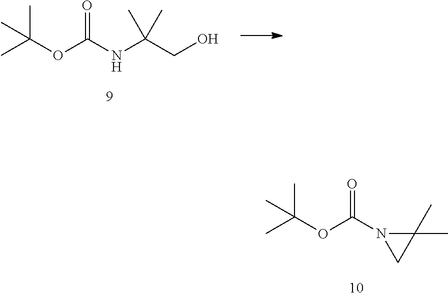

To a solution of tert-butyl N-(2-hydroxy-1,1-dimethyl-ethyl)carbamate (30 g, 155.35 mmol) in diethyl ether (750 mL) was added p-TsCl (35.6 g, 186.73 mmol) and powdered KOH (103 g, 1.5605 mol) at 0° C. The reaction temperature was raised to reflux temperature and stirred for 16 h. Another portion of KOH (17 g, 303 mmol) was added and the reaction was refluxed for another 2 h. The reaction was cooled to room temperature and diluted with diethyl ether (500 mL). The formed solid was removed by filtration through a glass fritted funnel and washed with more diethyl ether (100 mL). The combined ethereal filtrate was washed with water (100 mL) and brine (100 mL), dried over anhydrous magnesium sulfate, filtered and concentrated under reduced pressure to furnish as a clear oil, tert-butyl 2,2-dimethylaziridine-1-carboxylate (24.602 g, 88%). ¹H NMR (500 MHz, Chloroform-d) δ 2.04 (s, 2H), 1.46 (s, 9H), 1.28 (s, 6H) ppm.

### Step 2: tert-Butyl N-(1,1-dimethylpent-4-enyl)carbamate (Compound 11)

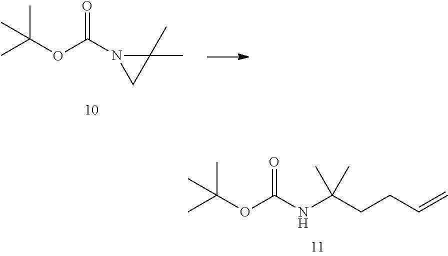

A reaction flask was charged with allyl(chloro)magnesium in THF (205 mL, 2 M, 410 mmol) and anhydrous THF (200 mL). The solution was cooled to −30° C. and copper(I) bromide (dimethyl sulfide complex) (28 g, 136.2 mmol) was added. The reaction mixture was stirred at the same temperature for 30 min, then cooled to −78° C. A solution of tert-butyl 2,2-dimethylaziridine-1-carboxylate (24.602 g, 136.49 mmol) in anhydrous THF (200 mL) was added to the reaction mixture dropwise. The reaction was stirred at the same temperature for 30 min, and then moved to a −20° C. freezer and stored for 3 h. The reaction was quenched with a saturated aqueous ammonium chloride solution (200 mL) at 0° C. The reaction was stirred at room temperature for 10 minutes, then diluted with diethyl ether (200 mL). The solution was filtered through a pad of Celite and washed with ether (100 mL). The two layers were separated, and the aqueous layer was extracted with diethyl ether (2×200 mL). The combined organic layers were washed with brine (200 mL), dried over anhydrous magnesium sulfate and concentrated under vacuum. The residue was purified by silica gel chromatography using a gradient from 0% to 10% diethyl ether in hexanes to furnish, as a light yellow liquid, tert-butyl N-(1,1-dimethylpent-4-enyl)carbamate (18.6 g, 61%). ¹H NMR (500 MHz, Chloroform-d) δ 5.82 (ddt, J 16.8, 10.2, 6.6, 6.6 Hz, 1H), 5.09-4.87 (m, 2H), 4.38 (s, 1H), 2.11-1.98 (m, 2H), 1.79-1.64 (m, 2H), 1.43 (s, 9H), 1.26 (s, 6H) ppm.

### Step 3: 2-Methylhex-5-en-2-amine Hydrochloride Salt (Compound 3-HCl)

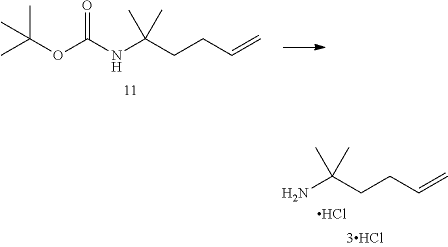

A solution of tert-butyl N-(1,1-dimethylpent-4-enyl)carbamate (26.6 g, 124.7 mmol) and HCl in diethyl ether (350 mL, 2 M, 700 mmol) was stirred at room temperature for 2 days. The solvent was removed and the residue was triturated with hexanes to furnish, as a white solid, 2-methylhex-5-en-2-amine (hydrochloride salt) (15.198 g, 77%). ¹H NMR (500 MHz, DMSO-d6) δ 8.08 (s, 3H), 5.92-5.64 (m, 1H), 5.15-4.87 (m, 2H), 2.21-1.96 (m, 2H), 1.72-1.49 (m, 2H), 1.23 (s, 6H) ppm.

### Intermediate 2: Methyl 6-Chloro-3-nitro-5-(trifluoromethyl)pyridine-2-carboxylate (Compound 16)

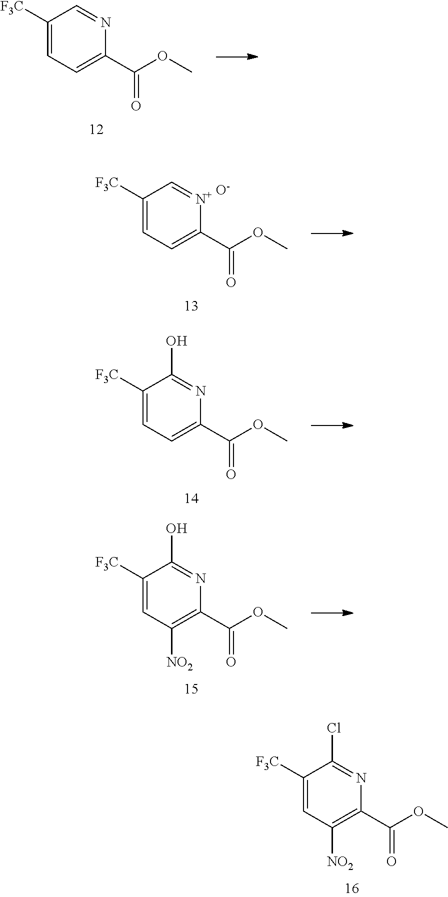

### Step 1: Methyl 1-Oxido-5-(trifluoromethyl)pyridin-1-ium-2-carboxylate (Compound 13)

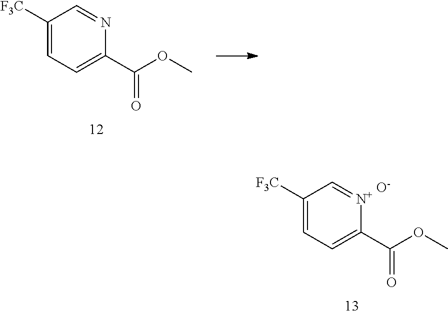

Urea hydrogen peroxide (62.7 g, 646.53 mmol) was added portion-wise to a stirred solution of methyl 5-(trifluoromethyl)pyridine-2-carboxylate (40 g, 191.09 mmol) in 1,2-dichloroethane (300 mL) at 0° C. Trifluoroacetic anhydride (107.70 g, 72 mL, 507.65 mmol) was then added over 30 minutes at a temperature of −10° C., with cooling bath (C₀₂/acetone bath). The reaction mixture was then stirred for a further 30 minutes at a temperature of 0° C. and then for 1 h at ambient temperature. The reaction mixture was then poured into cooled ice-water (600 mL). The mixture was diluted with dichloromethane (300 mL) and then layers were separated. The aqueous phase was extracted with dichloromethane (2×200 mL). The combined organic phase was washed with water (2×300 mL) and brine (1×200 mL), dried over anhydrous sodium sulfate, filtered and concentrated under reduced pressure to give as a light yellow solid, methyl 1-oxido-5-(trifluoromethyl)pyridin-1-ium-2-carboxylate (47.6 g, 90%). ¹H NMR (300 MHz, DMSO-d6) δ 8.89 (s, 1H), 8.02-7.90 (m, 1H), 7.86-7.72 (m, 1H), 3.89 (s, 3H) ppm. ¹⁹F NMR (282 MHz, DMSO-d6) δ −62.00 (s, 3F) ppm. ESI-MS m/z calc. 221.02998, found 222.1 (M+1)⁺; Retention time: 1.24 minutes (LC Method A).

### Step 2: Methyl 6-Hydroxy-5-(trifluoromethyl)pyridine-2-carboxylate (Compound 14)

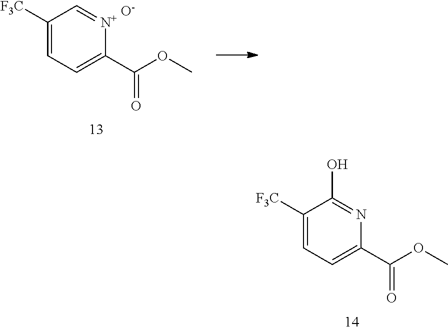

Trifluoroacetic anhydride (291.62 g, 193 mL, 1.3885 mol) was added dropwise to a mixture of methyl 1-oxido-5-(trifluoromethyl)pyridin-1-ium-2-carboxylate (51.058 g, 230.66 mmol) in DMF (305 mL) at 0° C. The mixture was then stirred at room temperature overnight. The mixture was concentrated under reduced pressure to remove excess of trifluoroacetic acid. The residual DMF solution was poured dropwise to a 0° C. cooled and stirring water volume (1000 mL). The precipitated solid was collected by filtration and then washed with water (300 mL). The solid was dried under vacuum to afford methyl 6-hydroxy-5-(trifluoromethyl)pyridine-2-carboxylate (45.24 g, 86%) as a white solid. ¹H NMR (300 MHz, CDCl₃) δ 7.90 (d, J 7.2 Hz, 1H), 7.03 (d, J 7.2 Hz, 1H), 4.02 (s, 3H) ppm. One exchangeable proton not observed in ¹H NMR. ¹⁹F NMR (282 MHz, CDCl₃) δ −66.39 (s, 3F) ppm. ESI-MS m/z calc. 221.03, found 222.1 (M+1)⁺; retention time: 1.43 minutes (LC Method A.

### Step 3: Methyl 6-Hydroxy-3-nitro-5-(trifluoromethyl)pyridine-2-carboxylate (Compound 15)

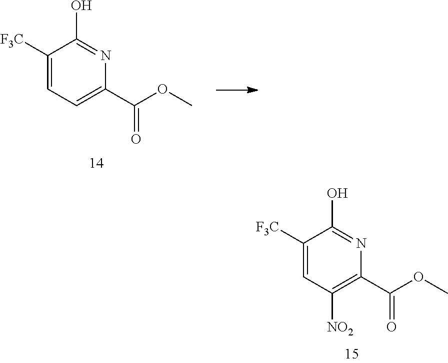 [SMILES: COC(=O)c1ccc(C(F)(F)F)c(O)n1 ; COC(=O)c1nc(O)c(C(F)(F)F)cc1[N+](=O)[O-]]

To an ice-cooled solution of methyl 6-hydroxy-5-(trifluoromethyl)pyridine-2-carboxylate (33.04 g, 149.41 mmol) in sulfuric acid (200 mL of 18.4 M, 3.6800 mol) was added nitric acid (13 mL of 15.8 M, 205.40 mmol) dropwise. After 5 min, the ice bath was removed, and the reaction mixture was stirred at 38° C. overnight. The reaction was not completed, so nitric acid (3 mL of 15.8 M, 47.400 mmol) was added dropwise at room temperature and the reaction was heated at 38° C. for 4.5 h. The reaction was poured slowly on ice-cold water (900 mL) and the mixture was cooled at 0° C. for 15 minutes. Then the resultant solid was isolated by filtration and washed with water (600 mL). The solid was dried overnight under vacuum to give, as a white solid, methyl 6-hydroxy-3-nitro-5-(trifluoromethyl)pyridine-2-carboxylate (39.49 g, 99%). ¹H NMR (300 MHz, DMSO-d6) δ 8.54 (s, 1H), 3.95 (s, 3H) ppm. One exchangeable proton not observed in ¹H NMR. ¹⁹F NMR (282 MHz, DMSO-d6) δ −64.56 (s, 3F) ppm. ESI-MS m/z calc. 266.0151, found 267.1 (M+1)⁺; retention time: 1.64 minutes (LC Method A).

### Step 4: Methyl 6-Chloro-3-nitro-5-(trifluoromethyl)pyridine-2-carboxylate (Compound 16)

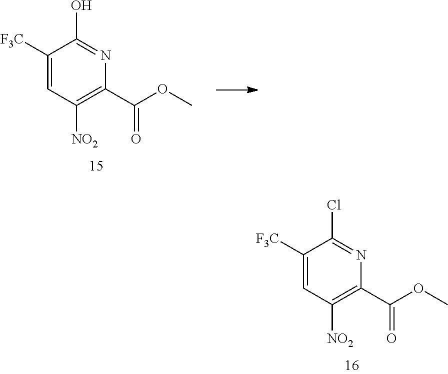 [SMILES: COC(=O)c1nc(O)c(C(F)(F)F)cc1[N+](=O)[O-] ; COC(=O)c1nc(Cl)c(C(F)(F)F)cc1[N+](=O)[O-]]

A mixture of methyl 6-hydroxy-3-nitro-5-(trifluoromethyl)pyridine-2-carboxylate (10 g, 37.575 mmol) and phenyl dichlorophosphate (48.008 g, 34 mL, 227.55 mmol) was heated at 170° C. for 90 minutes. After cooling to room temperature, the mixture was diluted with ethyl acetate (400 mL) and washed with brine (2×200 mL). The organic phase was dried on anhydrous sodium sulfate, filtered, and concentrated under reduced pressure. Purification by silica gel chromatography (0% to 15% of ethyl acetate in heptanes) provided methyl 6-chloro-3-nitro-5-(trifluoromethyl)pyridine-2-carboxylate (5.45 g, 50%) as a yellow solid. ¹H NMR (300 MHz, CDCl₃) δ 8.75 (s, 1H), 4.07 (s, 3H) ppm. ¹⁹F NMR (282 MHz, CDCl₃) δ −64.12 (s, 3F) ppm. ESI-MS m/z calc. 283.9812, found 285.0 (M+1)⁺; retention time: 1.95 minutes (LC Method A).

### Intermediate 3: Preparation of 2-Benzyloxy-2-(trifluoromethyl)pent-4-enehydrazide (hydrochloride salt) (Compound 22-HCl)

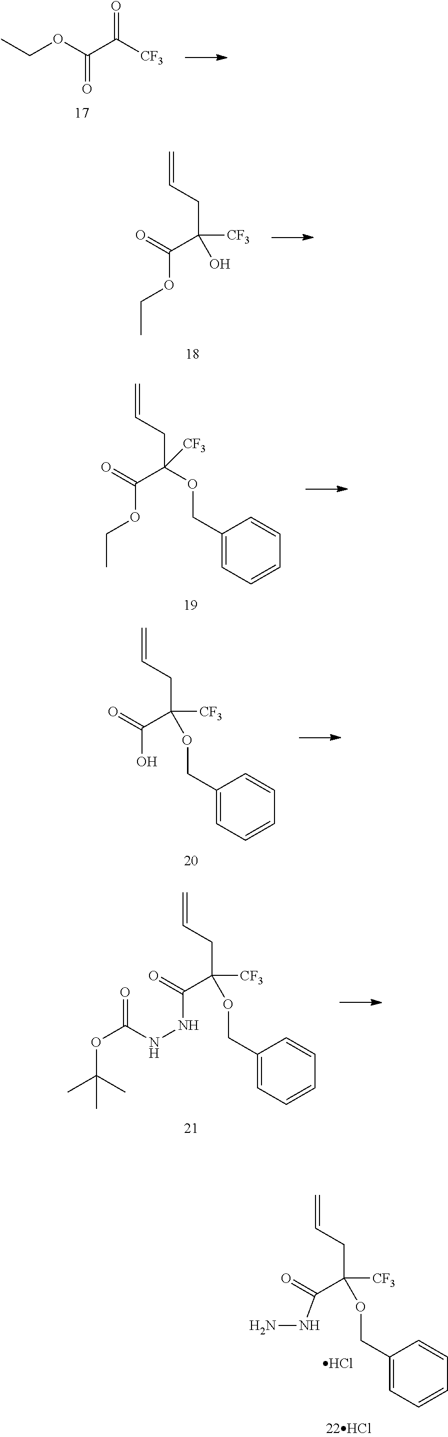

### Step 1: Ethyl 2-Hydroxy-2-(trifluoromethyl)pent-4-enoate (Compound 18)

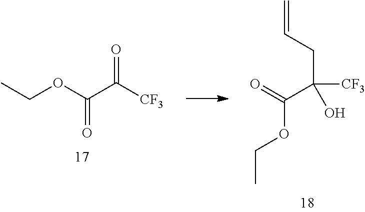

To a solution of ethyl 3,3,3-trifluoro-2-oxo-propanoate (30 g, 176.38 mmol) in diethyl ether (300 mL) at −78° C. was added allyl(bromo)magnesium (185 mL of 1 M, 185.00 mmol) dropwise over a period of 3 h (internal temperature: −74° C.-−76° C.). The mixture was stirred at −78° C. for 45 min. The dry ice-acetone bath was removed. The mixture was allowed to warm to about 10° C. over a period of 1 h and added to a mixture of 1 M aqueous HCl (210 mL) and crushed ice (400 g) (pH 4). The mixture was extracted with EtOAc, washed with 5% aqueous NaHCO₃, brine and dried over anhydrous Na₂SO₄. The mixture was filtered, concentrated and co-evaporated with hexane to give as a light yellow oil, ethyl 2-hydroxy-2-(trifluoromethyl)pent-4-enoate (42.2 g, 90%). ¹H NMR (300 MHz, CDCl₃) δ 1.33 (t, J 7.1 Hz, 3H), 2.60-2.79 (m, 2H), 3.84 (br. s., 1H), 4.24-4.48 (m, 2H), 5.09-5.33 (m, 2H), 5.59-5.82 (m, 1H) ppm. ¹⁹F NMR (282 MHz, CDCl₃) δ −78.5 (s, 3F) ppm.

### Step 2: Ethyl 2-Benzyloxy-2-(trifluoromethyl)pent-4-enoate (Compound 19)

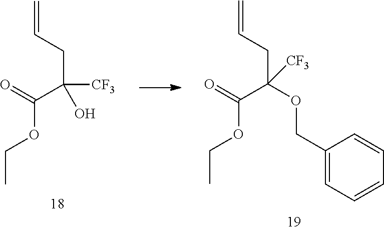 [SMILES: C=CCC(O)(C(=O)OCC)C(F)(F)F ; C=CCC(OCc1ccccc1)(C(=O)OCC)C(F)(F)F]

To a solution of ethyl 2-hydroxy-2-(trifluoromethyl)pent-4-enoate (18.56 g, 83.105 mmol) in DMF (100 mL) was added NaH (5.3 g, 60% w/w, 132.51 mmol) at 0° C. The reaction was stirred for 15 minutes and benzyl bromide (21.14 g, 15 mL, 121.12 mol) and tetrabutyl ammonium iodide (8.5 g, 23.012 mmol) were added. The mixture was stirred at room temperature overnight. The reaction was quenched with water (300 mL) and extracted with ethyl acetate (3×300 mL). The combined organic layers were washed with brine (500 mL) and dried over sodium sulfate. Purification by silica gel chromatography (20% to 60% DCM in hexanes) provided ethyl 2-benzyloxy-2-(trifluoromethyl)pent-4-enoate (22.01 g, 70%) as colorless oil. ¹H NMR (250 MHz, CDCl₃) δ 7.55-7.25 (m, 5H), 6.00-5.80 (m, 1H), 5.30-5.10 (m, 2H), 4.86 (d, J 10.5 Hz, 1H), 4.68 (d, J 10.5 Hz, 1H), 4.33 (q, J 7.0 Hz, 2H), 2.81 (d, J 7.0 Hz, 2H), 1.34 (t, J 7.1 Hz, 3H) ppm. ESI-MS m/z calc. 302.113, found 303.5 (M+1)⁺; retention time: 4.14 minutes (LC Method B).

### Step 3: 2-Benzyloxy-2-(trifluoromethyl)pent-4-enoic acid (Compound 20)

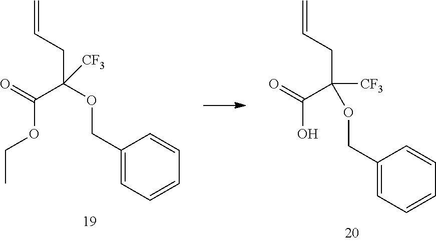 [SMILES: C=CCC(OCc1ccccc1)(C(=O)OCC)C(F)(F)F ; C=CCC(OCc1ccccc1)(C(=O)O)C(F)(F)F]

Into a solution of ethyl 2-benzyloxy-2-(trifluoromethyl)pent-4-enoate (28.99 g, 95.902 mmol) in methanol (150 mL) was added a solution of NaOH (7.6714 g, 191.80 mmol) in water (50 mL). The reaction mixture was stirred at 40° C. for 3 h. The reaction mixture was concentrated under vacuum, the residue was diluted with water (200 mL) and washed with diethyl ether (200 mL). The aqueous layer was acidified with concentrated HCl to pH 1 and extracted with diethyl ether (3×200 mL). The combined organic layers were washed with brine, dried over anhydrous sodium sulfate, and concentrated under vacuum to furnish, as a light yellow liquid, 2-benzyloxy-2-(trifluoromethyl)pent-4-enoic acid (28.04 g, 99%). ¹H NMR (250 MHz, CDCl₃) δ 7.55-7.28 (m, 5H), 5.97-5.69 (m, 1H), 5.33-5.17 (m, 2H), 4.95-4.66 (m, 2H), 2.91 (d, J 7.1 Hz, 2H) ppm. One exchangeable proton was not observed in ¹H NMR.

### Step 4: tert-Butyl N-[[2-benzyloxy-2-(trifluoromethyl)pent-4-enoyl]amino]carbamate (Compound 21)

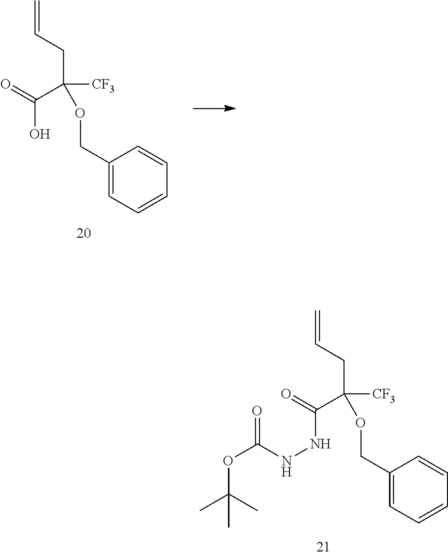 [SMILES: C=CCC(OCc1ccccc1)(C(=O)O)C(F)(F)F ; C=CCC(OCc1ccccc1)(C(=O)NNC(=O)OC(C)(C)C)C(F)(F)F]

To a solution of 2-benzyloxy-2-(trifluoromethyl)pent-4-enoic acid (300 g, 1.094 mol) in DMF (2 L) was added HATU (530 g, 1.394 mol) and DIEA (400 mL, 2.296 mol) and the mixture was stirred at ambient temperature for 10 min. To the mixture was added tert-butyl N-aminocarbamate (152 g, 1.150 mol) and the mixture stirred at ambient temperature for 36 h. The reaction was quenched with cold water (4 L) and the mixture extracted with EtOAc (2 ×2 L). The organic phase was washed brine, dried over MgSO₄, filtered and concentrated in vacuo. Purification by silica gel chromatography (0% to 40% EtOAc/hexanes) provided tert-butyl N-[[2-benzyloxy-2-(trifluoromethyl)pent-4-enoyl]amino]carbamate (386.49 g, 91%) as an oil that slowly crystallized to an off-white solid. ¹H NMR (400 MHz, DMSO) δ 10.00 (d, J 37.9 Hz, 1H), 8.93 (s, 1H), 7.46-7.39 (m, 2H), 7.38-7.29 (m, 3H), 6.01-5.64 (m, 1H), 5.32 (d, J 17.1 Hz, 1H), 5.17 (d, J 10.1 Hz, 1H), 4.77 (s, 2H), 2.96 (qd, J 15.4, 6.8 Hz, 2H), 1.39 (d, J 17.3 Hz, 9H) ppm. ESI-MS m/z calc. 388.16098, found 389.0 (M+1)⁺; Retention time: 2.51 minutes (LC Method C).

### Step 5: 2-Benzyloxy-2-(trifluoromethyl)pent-4-enehydrazide (Hydrochloride Salt) (Compound 22-HCl)

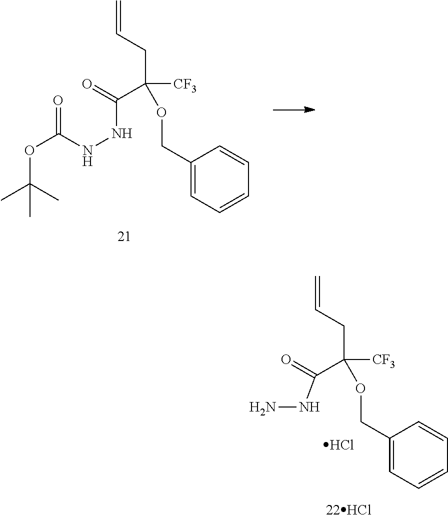 [SMILES: C=CCC(OCc1ccccc1)(C(=O)NNC(=O)OC(C)(C)C)C(F)(F)F ; C=CCC(OCc1ccccc1)(C(=O)NN)C(F)(F)F ; Cl]

To a solution of tert-butyl N-[[2-benzyloxy-2-(trifluoromethyl)pent-4-enoyl]amino]carbamate (98.5 g, 240.94 mmol) in DCM (400 mL) was added HCl in dioxane (200 mL of 4 M, 800.00 mmol). The mixture was stirred at room temperature for 2 h, concentrated and co-evaporated with DCM and hexanes to give 2-benzyloxy-2-(trifluoromethyl)pent-4-enehydrazide (hydrochloride salt) (81.15 g, 97%) as an off-white solid. ¹H NMR (500 MHz, DMSO-d6) δ 11.07 (s, 1H), 7.70-7.16 (m, 5H), 5.87-5.61 (m, 1H), 5.45-5.09 (m, 2H), 4.79 (s, 2H), 3.6-3.4 (m, 2H), 3.23-3.07 (m, 1H), 3.04-2.87 (m, 1H) ppm. ESI-MS m/z calc. 288.10855, found 289.2 (M+1); retention time: 2.0 minutes (LC Method D).

### Intermediate 4: Preparation of (2R)-2-Benzyloxy-2-(trifluoromethyl)pent-4-enehydrazide (Compound 23a)

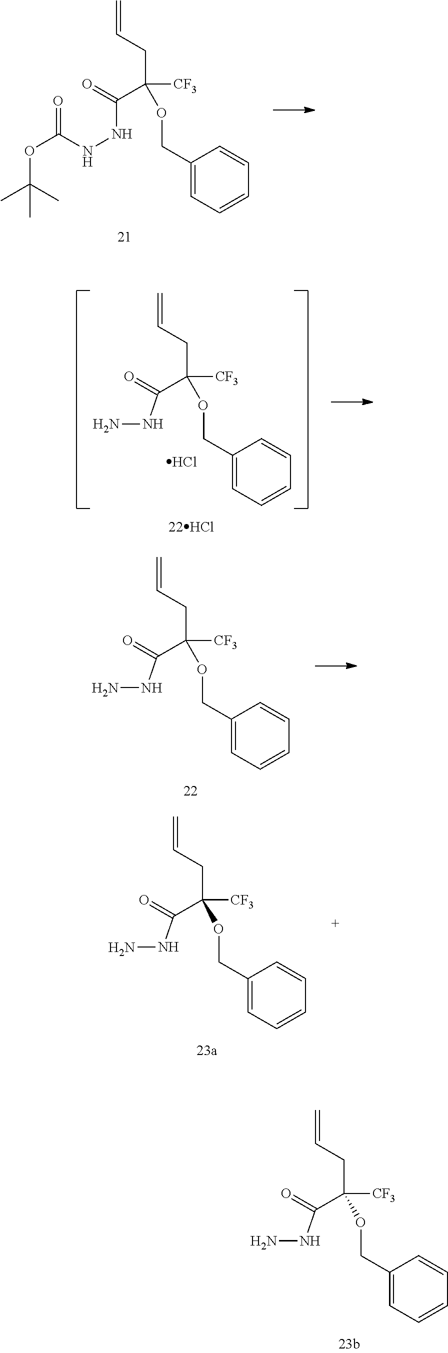

### Step 1: 2-Benzyloxy-2-(trifluoromethyl)pent-4-enehydrazide (Compound 22)

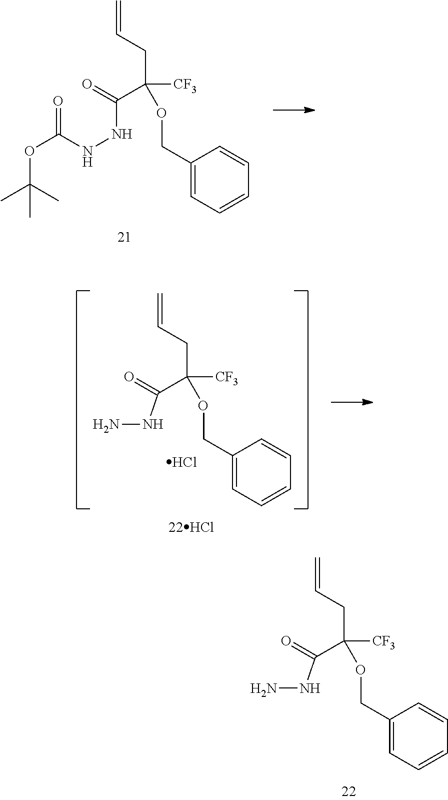

tert-Butyl N-[[2-benzyloxy-2-(trifluoromethyl)pent-4-enoyl]amino]carbamate (386.49 g, 995.1 mmol) was dissolved in DCM (1.25 L) and toluene (250 mL) and treated with HCl (750 mL of 4 M, 3.000 mol) at room temperature and the yellow solution was stirred at room temperature for 18 h. The mixture was concentrated in vacuo and diluted with EtOAc (2 L). The mixture was treated with NaOH (600 mL of 2 M, 1.200 mol) and stirred at ambient temperature for 10 min. The organic phase was separated, washed with 1 L of brine, dried over MgSO₄, filtered and concentrated in vacuo and used directly in the ensuing step (trace toluene present),

2-benzyloxy-2-(trifluoromethyl)pent-4-enehydrazide (286 g, 100%). ¹H NMR (400 MHz, DMSO) δ 9.34 (s, 1H), 7.40-7.22 (m, 5H), 5.69 (ddt, J 17.1, 10.3, 6.9 Hz, 1H), 5.33-5.23 (m, 1H), 5.15 (dd, J 10.3, 1.8 Hz, 1H), 4.73 (s, 2H), 4.51 (s, 2H), 3.05-2.87 (m, 2H) ppm. ESI-MS m/z calc. 288.10855, found 289.0 (M+1); retention time: 1.32 minutes (LC Method E).

### Step 2: (2R)-2-Benzyloxy-2-(trifluoromethyl)pent-4-enehydrazide (Compound 23a)

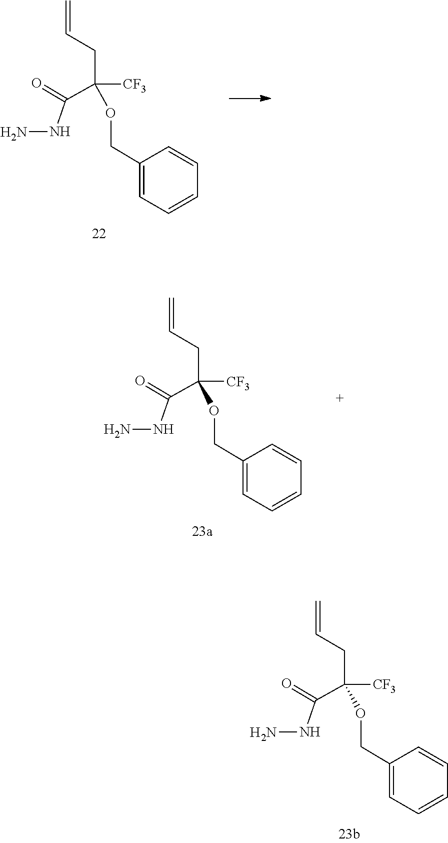 [SMILES: C=CCC(OCc1ccccc1)(C(=O)NN)C(F)(F)F ; C=CC[C@@](OCc1ccccc1)(C(=O)NN)C(F)(F)F ; C=CC[C@](OCc1ccccc1)(C(=O)NN)C(F)(F)F]

Racemic 2-benzyloxy-2-(trifluoromethyl)pent-4-enehydrazide (5.0 g, 17.35 mmol) was separated by chiral SFC using a ChiralPak IG column (250×21.2 mm; 5 m) at 40° C. using a mobile phase of 7% MeOH (plus 20 mM NH₃)/93% C₀₂ at a 70 mL/min flow and concentration of the sample was 111 mg/mL in methanol (no modifier), injection volume=160 L with an outlet pressure of 136 bar, detection wavelength of 210 nm providing two single enantiomer products:

The first enantiomer to elute was isolated as (2S)-2-benzyloxy-2-(trifluoromethyl)pent-4-enehydrazide (Compound 23a, 1.79 g, 72%). ¹H NMR (400 MHz, DMSO-d6) δ 9.31 (s, 1H), 7.45-7.39 (m, 2H), 7.38-7.26 (m, 3H), 5.77-5.62 (m, 1H), 5.28 (dq, J 17.1, 1.6 Hz, 1H), 5.15 (dq, J 10.2, 1.3 Hz, 1H), 4.72 (s, 2H), 4.44 (d, J 4.2 Hz, 2H), 2.99 (dd, J 7.4, 1.3 Hz, 1H), 2.91 (dd, J 15.4, 6.4 Hz, 1H) ppm. ESI-MS m/z calc. 288.10855, found 289.2 (M+1)⁺; retention time: 1.28 minutes (LC Method F).

The second enantiomer to elute was isolated as (2R)-2-benzyloxy-2-(trifluoromethyl)pent-4-enehydrazide (Compound 23b, 1.7 g, 68%) as a white solid. ¹H NMR (400 MHz, DMSO-d6) δ 9.31 (s, 1H), 7.48-7.39 (m, 2H), 7.39-7.25 (m, 3H), 5.77-5.62 (m, 1H), 5.28 (dq, J 17.1, 1.6 Hz, 1H), 5.15 (dq, J 10.2, 1.5 Hz, 1H), 4.73 (s, 2H), 4.51 (s, 2H), 3.00 (dd, J 15.3, 7.5 Hz, 1H), 2.91 (dd, J 15.3, 6.4 Hz, 1H) ppm. ESI-MS m/z calc. 288.10855, found 289.2 (M+1)⁺; retention time: 1.28 minutes (LC Method F).

### Example 5: Synthesis of (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I)

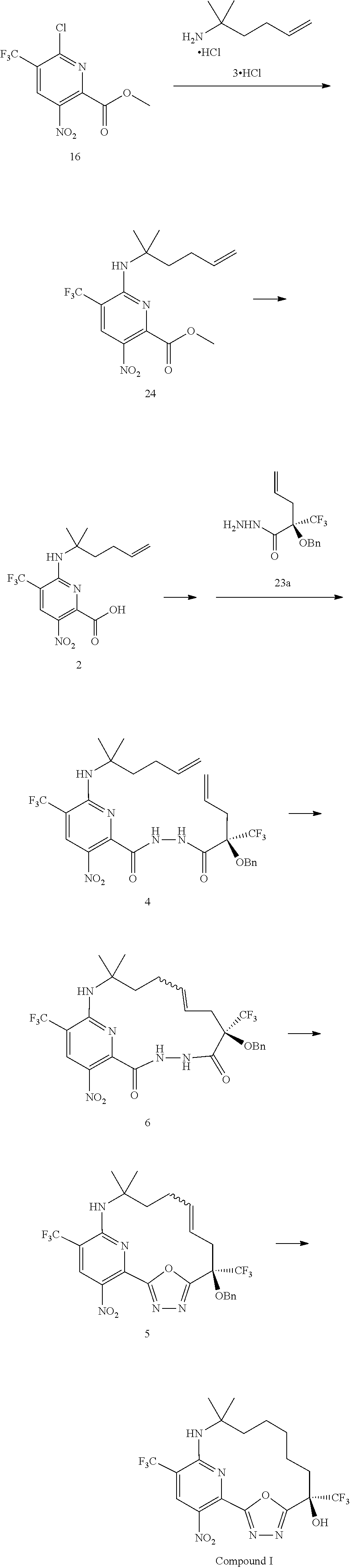

### Step 1: Methyl 6-((2-Methylhex-5-en-2-yl)amino)-3-nitro-5-(trifluoromethyl)picolinate (Compound 24)

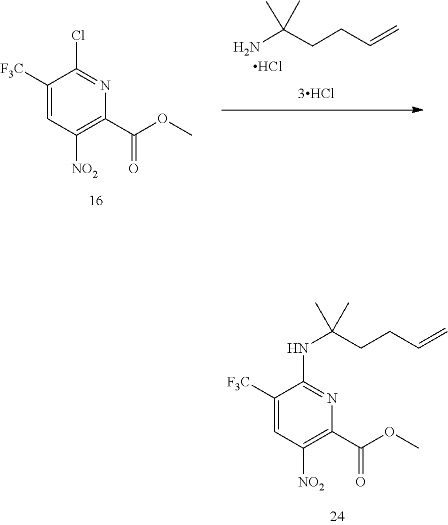

NaHCO₃ (88.6 g, 1.05 mol, 3 eq) was added to a mixture of Compound 16 (100 g, 0.35 mol, 1 eq) and Compound 3 HCl salt (57.9 g, 0.39 mol, 1.1 eq) in 2-MeTHF (800 mL, 8 vol). The mixture was heated to 75° C. (reaction mixture temp). After complete reaction by LC analysis, the mixture was allowed to cool to ambient temperature. At ambient temperature, 700 mL of water added. After stirring, the phases were separated. The organic layer was washed with 400 mL water, dried with Na₂SO₄, filtered, and concentrated. The crude product was dried under vacuum and used as-is for the next step. ESI-MS m/z calc. 361.12, found 362 (M+1)⁺. ¹H NMR (400 MHz, Chloroform-d) δ 8.45 (d, J=0.8 Hz, 1H), 5.77 (ddt, J=16.8, 10.2, 6.3 Hz, 1H), 5.51 (s, 1H), 5.01 (dq, J=17.1, 1.6 Hz, 1H), 4.98-4.92 (m, 1H), 4.01 (s, 3H), 2.11-2.00 (m, 2H), 2.01-1.92 (m, 2H), 1.88 (s, 1H), 1.49 (s, 6H). ¹⁹F NMR (376 MHz, CDCl₃) δ −64.47.

### Step 2: 6-((2-Methylhex-5-en-2-yl)amino)-3-nitro-5-(trifluoromethyl)picolinic acid (Compound 2)

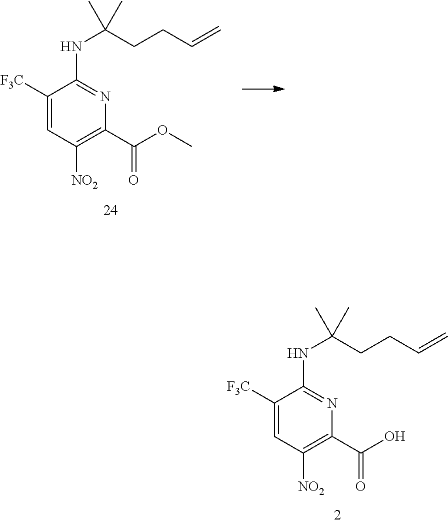 [SMILES: C=CCCC(C)(C)Nc1nc(C(=O)OC)c([N+](=O)[O-])cc1C(F)(F)F ; C=CCCC(C)(C)Nc1nc(C(=O)O)c([N+](=O)[O-])cc1C(F)(F)F]

To a solution of Compound 24 (54 g, 149.454 mmol, 1 equiv.) in EtOH (216 mL, 0.692 M, 4 Vols) was added 6 M NaOH (32.382 mL, 194.29 mmol, 1.3 equiv.) dropwise over 10 minutes via addition funnel, maintaining a temperature of <30° C. The mixture was stirred at ambient temperature until complete reaction by LC analysis. (If reaction does not convert further, add an additional 6 M NaOH (2.491 mL, 14.945 mmol, 0.1 equiv.) until reaction is completed.) The mixture was concentrated to remove EtOH. The mixture was concentrated and IPAc (420 mL, 8 vol) was added. The mixture was acidified by adding 6 M HCl (38.609 mL, 231.653 mmol, 1.55 equiv.) dropwise via addition funnel, maintaining the temperature at <30° C. Stirred for 10 min and allowed phases to separate. Added 26 g silica gel and 26 g activated carbon to the organic layer and stirred at ambient temperature for a few hours. The mixture was filtered over a pad of diatomaceous earth and washed twice with 25 mL IPAc. The filtrate was concentrated to an oil. Added 140 mL heptane and concentrated, and then repeated. Added 220 mL heptane to afford a slurry that was allowed to stir for NLT 2 h. The solid was collected by filtration and washed three times with 25 mL heptane. The solid was dried under vacuum to afford Compound 2 (42.3 g, 81.5%) as a light yellow solid. ESI-MS m/z calc. 347.11, found 348 (M+1)⁺. ¹H NMR (400 MHz, Chloroform-d) δ 8.47 (d, J=0.8 Hz, 1H), 7.89 (s, 2H), 5.78 (ddt, J=16.6, 10.2, 6.2 Hz, 1H), 5.56 (s, 1H), 5.12-4.89 (m, 2H), 2.15-1.93 (m, 4H), 1.53 (s, 6H). ¹⁹F NMR (376 MHz, CDCl₃) δ −64.45.

### Step 3: (R)-N′-(2-(Benzyloxy)-2-(trifluoromethyl)pent-4-enoyl)-6-((2-methylhex-5-en-2-yl)amino)-3-nitro-5-(trifluoromethyl)picolinohydrazide (Compound 4)

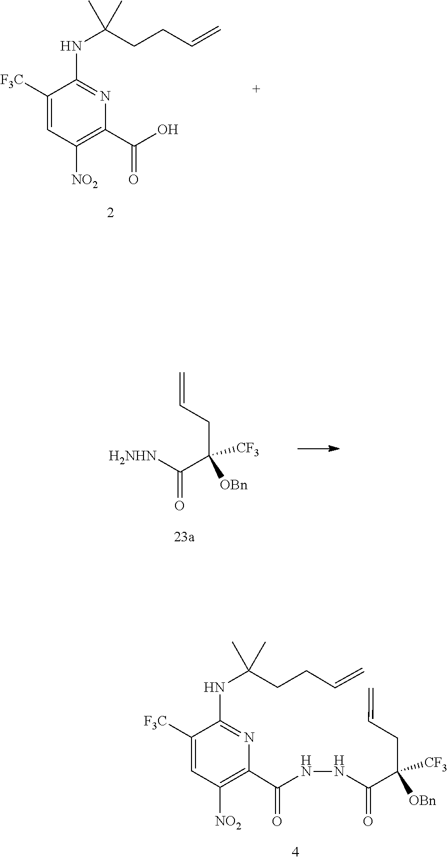

Compound 2 (900 g, 2591.496 mmol, 1 equiv.) was added into a reactor followed by N,N-dimethylformamide (2700 mL, 0.96 M, 3 Vols). The mixture was stirred at ambient temperature and 4-methylmorpholine (NMM, 288.34 g, 313.413 mL, 0.92 g/mL, 2850.646 mmol, 1.1 equiv.) was added dropwise. The mixture was stirred at ambient temperature for 15 minutes after complete addition. CDMT (500.488 g, 2850.646 mmol, 1.1 equiv.) was added as a slurry in N,N-dimethylformamide (900 mL, 2.879 M, 1 Vols). After addition the mixture was stirred for 1 h, Compound 23a (784.391 g, 2721.071 mmol, 1.05 equiv.) in N,N-dimethylformamide (900 mL, 2.879 M, 1 Vols) was then added to the mixture over 1.5 h. The mixture was stirred at ambient temperature until complete reaction was confirmed by LC analysis. Slowly added 2 L of water to quench the reaction and then added MTBE (12 L, 0.288 M, 13 Vols). Additional H₂O (14400 mL, 0.18 M, 16 Vols) was added. After stirring, the phases were separated. The organic layer was washed with 0.5 M NaOH (5400 mL, 6 Vols), 0.5 M HCl (5400 mL, 6 Vols), and 5% w/v aq NaCl (5400 mL, 6 Vols). The organic layer was dried over Na₂SO₄, filtered, and concentrated by rotary evaporation (40° C., 20 mbar). Added 2 L ethyl acetate to chase the MTBE and was concentrated on a rotary evaporator at 50° C. to afford crude Compound 4 (1551 g, 91.9%) that was used directly in the next step. ESI-MS m/z calc. 617.21, found 618 (M+1)⁺. ¹H NMR (400 MHz, Chloroform-d) δ 9.26 (s, 1H), 8.84 (d, J=4.6 Hz, 1H), 8.33-8.26 (m, 1H), 7.47-7.29 (m, 6H), 5.81 (dddd, J=27.2, 12.3, 10.2, 6.7 Hz, 2H), 5.45 (s, 1H), 5.38-5.27 (m, 2H), 5.01 (dq, J=17.2, 1.6 Hz, 1H), 4.95 (dt, J=10.2, 1.4 Hz, 1H), 4.85 (s, 2H), 3.19-2.95 (m, 2H), 2.12-1.95 (m, 4H), 1.61 (s, 5H), 1.51 (s, 6H). ¹⁹F NMR (376 MHz, CDCl₃) δ −64.53, −73.76.

### Step 4: (R)-9-(Benzyloxy)-3,3-dimethyl-1⁵-nitro-1³,9-bis(trifluoromethyl)-2,11,12-triaza-1(2,6)-pyridinacyclotridecaphan-6-ene-10,13-dione (Compound 6)

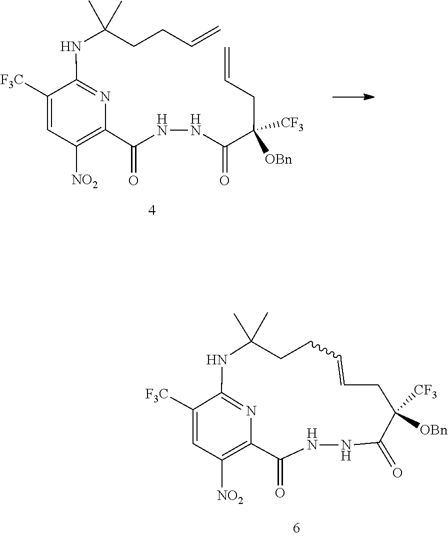 [SMILES: C=CCCC(C)(C)Nc1nc(C(=O)NNC(=O)[C@@](CC=C)(OCc2ccccc2)C(F)(F)F)c([N+](=O)[O-])cc1C(F)(F)F ; CC1(C)CCC=CC[C@](OCc2ccccc2)(C(F)(F)F)C(=O)NNC(=O)c2nc(c(C(F)(F)F)cc2[N+](=O)[O-])N1]

A solution of Compound 4 (517 g, 837.181 mmol, 1 equiv.) in EtOAc (77.55 L, 0.011 M, 150 Vols) was stirred at ambient temperature and sub-surface N2 sparging was started. After 30 min of sparging, dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zhan Catalyst-1B, 61.428 g, 83.718 mmol, 0.1 equiv.) was added and the mixture was heated to 40° C. while continuing sub-surface N2 sparging. After complete reaction by LC analysis, the mixture was cooled to ambient temperature. 2-Mercaptonicotinic acid (64.954 g, 418.59 mmol, 0.5 equiv.) followed by TEA (42.357 g, 58.829 mL, 0.72 g/mL, 418.59 mmol, 0.5 equiv.) were then charged to the reactor and the mixture stirred overnight. Added silica gel (1265.817 g, 5023.083 mmol, 6 equiv.) to mixture and stirred at least one hour. The mixture was filtered through a glass filter with a layer of silica gel (2 kg) and a thin layer of diatomaceous earth, washing the solids with EtOAc (20.68 L, 0.04 M, 40 Vols). Distilled off solvent from the filtrate via a rotary evaporator. Final weight for crude material was 397 g with a purity of 79.71% area percent by LC analysis.

A slurry of combined crude material (Compound 4, 1551 g, 2383.955 mmol, 1 equiv.) in MTBE (6.204 L, 0.384 M, 4 Vols) was heated to 55° C. and stirred for 1 h. MTBE (1.241 L, 1.921 M, 0.8 Vols) and heptane (4963.2 mL, 0.48 M, 3.2 Vols) were added over 15 minutes. The mixture was stirred for an additional 30 minutes and then cooled to 10° C. over 2 h, followed by stirring overnight at 10° C.

The mixture was then filtered and the filter cake was first washed with MTBE (310.2 mL, 0.2 Vols) and then heptane (3×413 mL, 0.8 Vols). Pulled the filter cake dry for 1 h then transferred to the rotary evaporator to dry at 50° C. for 4 h to afford Compound 6 (1119 g, 78.4%). ESI-MS m/z calc. 589.18, found 590 (M+1)⁺. ¹H NMR (400 MHz, Chloroform-d) δ 8.51 (d, J=8.9 Hz, 1H), 7.90 (s, 1H), 7.51 (s, 1H), 7.48-7.39 (m, 1H), 7.39-7.29 (m, 2H), 7.13-6.97 (m, 2H), 5.67 (ddd, J=14.9, 10.8, 3.9 Hz, 1H), 5.42-5.32 (m, 1H), 4.76-4.55 (m, 2H), 4.43 (d, J=9.9 Hz, 1H), 2.90-2.72 (m, 2H), 2.55 (t, J=12.6 Hz, 1H), 2.19-2.06 (m, 1H), 2.05 (s, 1H), 1.59 (s, 5H), 1.52 (s, 4H), 1.47-1.39 (m, 1H), 1.32-1.19 (m, 4H). ¹⁹F NMR (376 MHz, CDCl₃) δ −64.25, −64.62, −73.96, −74.27.

### Step 5: (R)-10-(Benzyloxy)-4,4-dimethyl-2³-nitro-2⁵,10-bis(trifluoromethyl)-3-aza-1(2,5)-oxadiazola-2(2,6)-pyridinacyclodecaphan-7-ene (Compound 5)

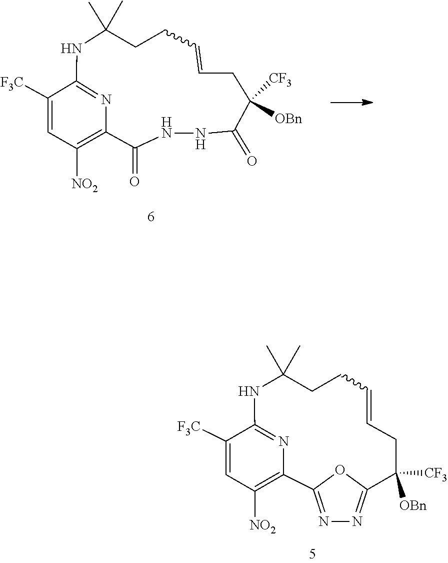 [SMILES: CC1(C)CCC=CC[C@](OCc2ccccc2)(C(F)(F)F)C(=O)NNC(=O)c2nc(c(C(F)(F)F)cc2[N+](=O)[O-])N1 ; CC1(C)CCC=CC[C@](OCc2ccccc2)(C(F)(F)F)c2nnc(o2)-c2nc(c(C(F)(F)F)cc2[N+](=O)[O-])N1]

A solution of Compound 6 (20.0 g, 33.9 mmol, 1 equiv.) and 1,4-diazabicyclo[2.2.2]octane (DABCO; 5.71 g, 50.9 mmol, 1.5 equiv.) in DCM (160 mL, 8 VolEq) was stirred at RT when 25 w/v % 2-chloro-1,3-dimethylimidazolinium chloride (DMC; 6.23 g, 24.9 mL, 37.3 mmol, 1.1 equiv.) was added over 5 min while maintaining the reaction temperature between 15-30° C. Immediately after complete addition of DMC, the reaction suspension was diluted with PhMe (40 mL, 2 VolEq) and concentrated to remove most of the DCM. The concentrate was diluted again with PhMe (160 mL, 8 VolEq) total volume. The suspension was heated at 100° C. The reaction temperature was maintained at 100-105° C. for 5-6 h until complete reaction was confirmed by LC analysis. The suspension was cooled to RT and the solid (DABCO·HCl and N,N-dimethylimidazolidinone) was removed by filtration. The filter-cake was washed with MTBE (40 mL, 2 Vols) twice. The filtrate and washings were combined and washed sequentially, first with water (60 mL, 3 VolEq), then 0.5 M HCl (67.9 mL, 33.9 mmol, 1 equiv.), and then water (60 mL, 3 VolEq). The mixture was then concentrated to afford crude Compound 5 (20.9 g; 108% theory).

The crude Compound 5 was dissolved in hot (75° C.) EtOH (40 mL) and cooled to RT over 1 h. The solid was collected by filtration and the filter-cake was washed with EtOH (2×10-mL) and then air-dried to afford Compound 5 (14.0 g; 72%) as a yellow solid. ESI-MS m/z calc. 571.17, found 572 (M+1)⁺. ¹H NMR (400 MHz, Chloroform-d) δ 8.47 (d, J=0.9 Hz, 1H), 7.41-7.21 (m, 7H), 5.76-5.58 (m, 2H), 5.56-5.47 (m, 1H), 4.85 (dd, J=11.2, 1.5 Hz, 1H), 4.52 (d, J=11.0 Hz, 1H), 3.14-3.03 (m, 1H), 2.69 (dd, J=14.3, 8.5 Hz, 1H), 2.52 (td, J=9.9, 9.5, 5.6 Hz, 1H), 2.14 (ddd, J=12.0, 10.0, 5.4 Hz, 1H), 1.99 (ddt, J=14.7, 8.0, 4.0 Hz, 2H), 1.45 (s, 3H), 1.42 (s, 3H). ¹⁹F NMR (376 MHz, CDCl₃) δ −64.13, −72.99.

### Step 6: (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound 1)

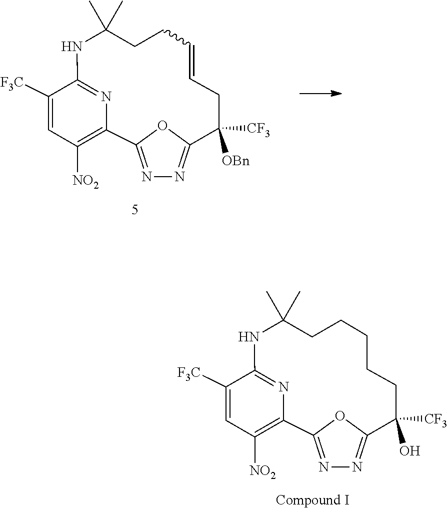 [SMILES: CC1(C)CCC=CC[C@](OCc2ccccc2)(C(F)(F)F)c2nnc(o2)-c2nc(c(C(F)(F)F)cc2[N+](=O)[O-])N1 ; CC1(C)CCCCC[C@](O)(C(F)(F)F)c2nnc(o2)-c2nc(c(C(F)(F)F)cc2[N+](=O)[O-])N1]

A suspension of Compound 5 (14.0 g, 24.5 mmol, 1 equiv.) and 10 wt % palladium on activated carbon (Evonik Nobylst P1173; 2.6 g, 50 w/w %, 0.5 equiv.) in EtOH (210 mL, 15 VolEq), EtOAc (70 mL, 5 VolEq), and 7 M NH₃/MeOH (3.5 mL, 24.5 mmol, 1 equiv.) was evacuated and backfilled with N2 three times. The reaction vessel was evacuated and backfilled with H₂ (balloon pressure) and was rapidly-stirred at RT for 16 h. The reaction vessel was evacuated/backfilled with N2 three and the mixture was analyzed by LC for reaction completion. A portion of diatomaceous earth (2 g) was added to the reaction mixture and the suspension was filtered through a 1-cm diatomaceous earth-packed bed to remove the catalyst. The flask/filter-bed was washed with EtOH (3×10 mL) and the washings were combined with the filtrate and concentrated (45° C./20 torr) to afford Compound I (11.5 g; 104% theory) as a bright yellow foam.

The foam was dissolved in DCM (98 mL, 7 VolEq), filtered through a SiO₂-packed bed (14 g; 1 g/g). The filter-bed was washed with DCM (80 mL) and then 20% EtOAc/DCM (2×100 mL). Each subsequent washing became less colored, with the final wash being nearly colorless. The filtrate and washings were combined and concentrated to afford Compound I (11.1 g) as an orange solid. The solid was dissolved in warm (30° C.) DCM (98 mL, 7 VolEq) and diluted with heptane (98 mL, 7 VolEq) with rapid mixing. The solution was concentrated (45° C./360 torr), to remove most of the DCM, and then the resultant suspension was backfilled with an additional portion of heptane (98 mL, 7 Vols). The suspension was partially concentrated again (to remove residual DCM) and cooled to RT. The solid was collected by filtration and the filter-cake was washed with heptane (2×10-mL) and air-dried to afford Compound I (10.7 g; 96%) as a bright yellow, granular solid. ESI-MS m/z calc. 453.16, found 454 (M+1)⁺. ¹H NMR (400 MHz, DMSO-d6) δ 7.62 (s, 1H), 7.59 (s, 1H), 5.96 (s, 2H), 4.64 (s, 1H), 2.81 (p, J=7.2, 6.8 Hz, 1H), 2.28-2.15 (m, 1H), 2.06 (t, J=12.4 Hz, 1H), 1.82 (dt, J=12.1, 7.5 Hz, 1H), 1.65 (d, J=13.7 Hz, 1H), 1.44 (q, J =8.7, 8.0 Hz, 5H), 1.36 (s, 3H), 1.31 (s, 3H). ¹⁹F NMR (376 MHz, DMSO) δ −62.34, −78.23.

### Example 6: Alternative synthesis of (R)-10-(benzyloxy)-4,4-dimethyl-2³-nitro-2⁵,10-bis(trifluoromethyl)-3-aza-1(2,5)-oxadiazola-2(2,6)-pyridinacyclodecaphan-7-ene (Compound 5)

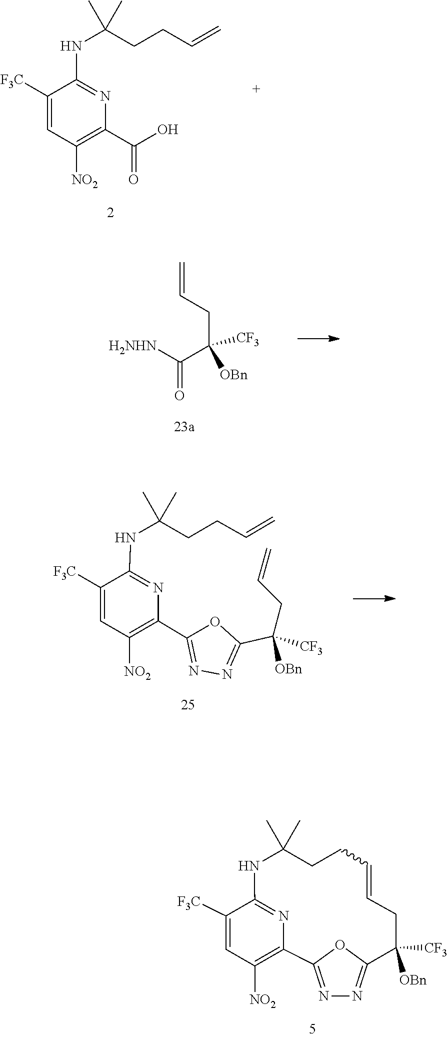

### Step 1: (R)-6-(5-(2-(benzyloxy)-1,1,1-trifluoropent-4-en-2-yl)-1,3,4-oxadiazol-2-yl)-N-(2-methylhex-5-en-2-yl)-5-nitro-3-(trifluoromethyl)pyridin-2-amine (Compound 25)

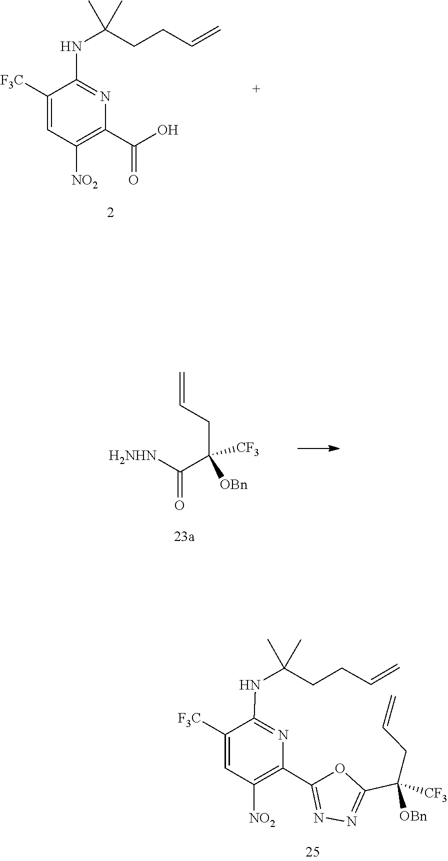

(R)-6-(5-(2-(Benzyloxy)-1,1,1-trifluoropent-4-en-2-yl)-1,3,4-oxadiazol-2-yl)-N-(2-methylhex-5-en-2-yl)-5-nitro-3-(trifluoromethyl)pyridin-2-amine (Compound 25) can be synthesized using a procedure similar to those reported in:
Li, C.; Dickson, H.D. Tett. Lett. 2009, 50, 6435.
Augustine, J.K.; Vairaperumal, V.; Narasimhan, S.; Alagarsamy, P.;
Radhakrishnan, A. Tetrahedron, 2009, 65, 9989.

A suitable vessel with overhead stirring and nitrogen inlet is charged with 6-((2-methylhex-5-en-2-yl)amino)-3-nitro-5-(trifluoromethyl)picolinic acid (14.9 mmol, 1 eq.), (R)-2-(benzyloxy)-2-(trifluoromethyl)pent-4-enehydrazide (15.6 mmol, 1.05 eq.), and acetonitrile (34.8 mL, 6 vol). The mixture is agitated, and T3P (33.1 g, 50 w/w % as a solution, 52.0 mmol, 3.5 eq) and DIPEA (89.2 mmol, 6 eq.) are charged to the reactor. The mixture is heated to an internal temperature of 78° C. and monitored by HPLC until desired reaction conversion is observed. The temperature is lowered to 25° C. and water (29 mL, 5 vol) is charged to the reactor, and the mixture is stirred for 10 minutes. MTBE (46.4 mL, 8 vol) is charged, stirred for 10 minutes, allowed to settle, and then separated. The organic layer is isolated and subsequently washed with 5% citric acid (29 mL, 5 vol), saturated sodium bicarbonate (29 mL, 5 vol), and water (29 mL, 5 vol). The organic layer is concentrated to an oil and used as is in subsequent processing.

### Step 2: (R)-10-(benzyloxy)-4,4-dimethyl-2³-nitro-2⁵,10-bis(trifluoromethyl)-3-aza-1(2,5)-oxadiazola-2(2,6)-pyridinacyclodecaphan-7-ene (Compound 5)

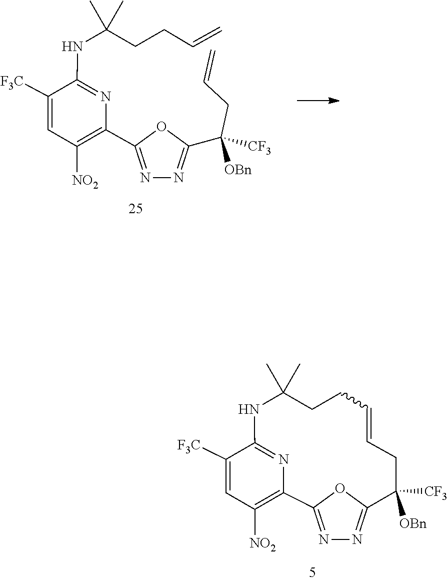 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)c2nnc(o2)-c2nc(c(C(F)(F)F)cc2[N+](=O)[O-])N1]

Compound 5 can be produced from Compound 25 by employing a method analogous to the one described in Step 4 of Example 5.

### Example 7: Alternative synthesis of (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I)

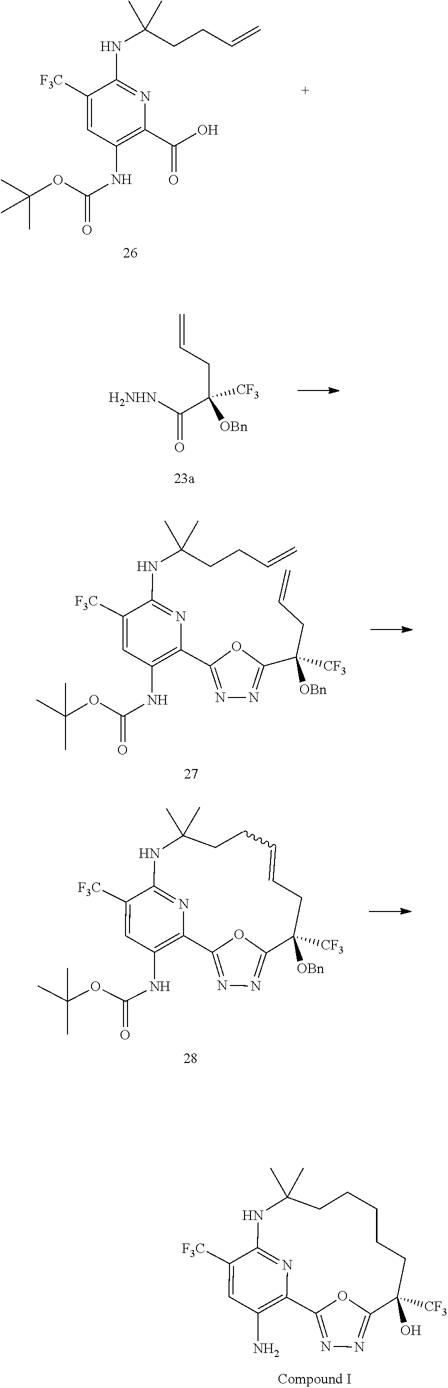

### Step 1: tert-Butyl (R)-(2-(5-(2-(benzyloxy)-1,1,1-trifluoropent-4-en-2-yl)-1,3,4-oxadiazol-2-yl)-6-((2-methylhex-5-en-2-yl)amino)-5-(trifluoromethyl)pyridin-3-yl)carbamate (Compound 27)

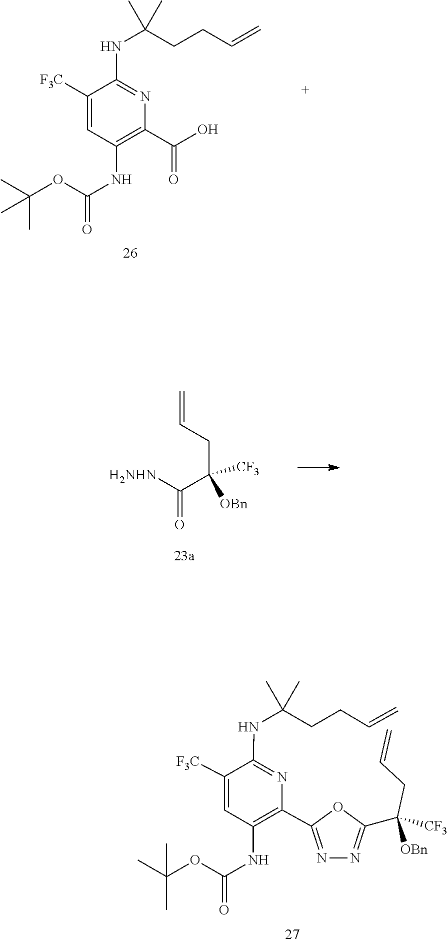

tert-Butyl (R)-(2-(5-(2-(benzyloxy)-1,1,1-trifluoropent-4-en-2-yl)-1,3,4-oxadiazol-2-yl)-6-((2-methylhex-5-en-2-yl)amino)-5-(trifluoromethyl)pyridin-3-yl)carbamate (Compound 27) can be synthesized using a procedure similar to those reported in:
Li, C.; Dickson, H. D. Tett. Lett. 2009, 50, 6435.
Augustine, J. K.; Vairaperumal, V.; Narasimhan, S.; Alagarsamy, P.; Radhakrishnan, A. Tetrahedron, 2009, 65, 9989.

A suitable vessel with overhead stirring and nitrogen inlet is charged with 3-((tert-butoxycarbonyl)amino)-6-((2-methylhex-5-en-2-yl)amino)-5-(trifluoromethyl)picolinic acid (Compound 26) (14.9 mmol, 1 eq.; prepared according to Example 90, Step 3 of WO2022/109573 A1), (R)-2-(benzyloxy)-2-(trifluoromethyl)pent-4-enehydrazide (15.6 mmol, 1.05 eq.), and acetonitrile (34.8 mL, 6 vol). The mixture is agitated, and T3P (33.1 g, 50 w/w % as a solution, 52.0 mmol, 3.5 eq) and DIPEA (89.2 mmol, 6 eq.) are charged to the reactor. The mixture is heated to an internal temperature of 78° C. and monitored by HPLC until desired reaction conversion is observed. The temperature is lowered to 25° C. and water (29 mL, 5 vol) is charged to the reactor, and the mixture is stirred for 10 minutes. MTBE (46.4 mL, 8 vol) is charged, stirred for 10 minutes, allowed to settle, and then separated. The organic layer is isolated and subsequently washed with 5% citric acid (29 mL, 5 vol), saturated sodium bicarbonate (29 mL, 5 vol), and water (29 mL, 5 vol). The organic layer is concentrated to an oil and used as is in subsequent processing.

### Step 2: tert-Butyl (R)-(10-(benzyloxy)-4,4-dimethyl-2⁵,10-bis(trifluoromethyl)-3-aza-1(2,5)-oxadiazola-2(2,6)-pyridinacyclodecaphan-7-en-2³-yl)carbamate (Compound 28)

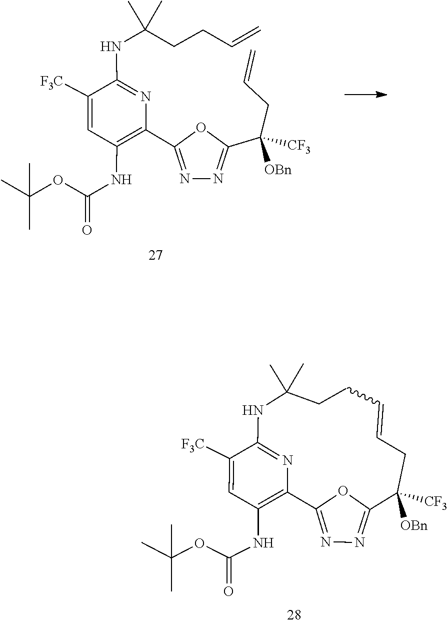 [SMILES: C=CCCC(C)(C)Nc1nc(-c2nnc([C@@](CC=C)(OCc3ccccc3)C(F)(F)F)o2)c(NC(=O)OC(C)(C)C)cc1C(F)(F)F ; CC1(C)CCC=CC[C@](OCc2ccccc2)(C(F)(F)F)c2nnc(o2)-c2nc(c(C(F)(F)F)cc2NC(=O)OC(C)(C)C)N1]

Compound 28 can be produced from Compound 27 by employing a method analogous to the one described in Step 4 of Example 5.

### Step 3: (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (Compound I)

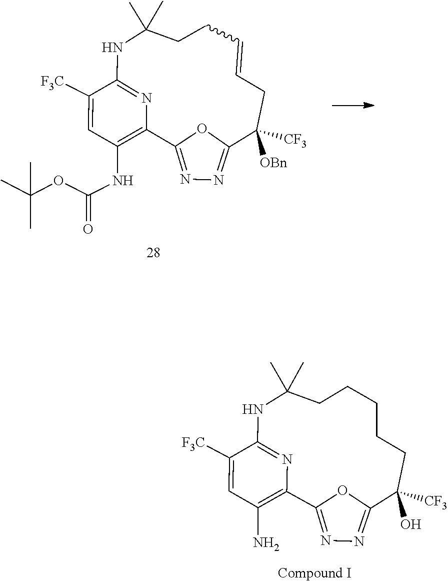 [SMILES: CC1(C)CCC=CC[C@](OCc2ccccc2)(C(F)(F)F)c2nnc(o2)-c2nc(c(C(F)(F)F)cc2NC(=O)OC(C)(C)C)N1 ; CC1(C)CCCCC[C@](O)(C(F)(F)F)c2nnc(o2)-c2nc(c(C(F)(F)F)cc2N)N1]

Compound I can be produced by Compound 28 by employing a method analogous to the one described in Step 6 of Example 5.

### OTHER EMBODIMENTS

All publications and patents referred to in this disclosure are incorporated herein by reference in their entirety to the same extent as if each individual publication or patent application were specifically and individually indicated to be incorporated by reference in their entirety. Should the meaning of the terms in any of the patents or publications incorporated by reference conflict with the meaning of the terms used in this disclosure, the meaning of the terms defined in this disclosure is intended to be controlling.

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. Compound I 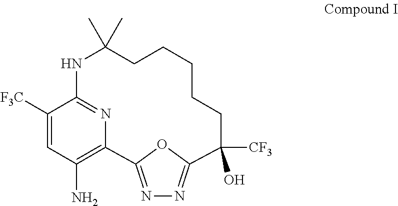 [SMILES: CC1(C)CCCCC[C@](O)(C(F)(F)F)C2=NN=C(O2)c2nc(c(C(F)(F)F)cc2N)N1]
as substantially amorphous Compound I.

2. The substantially amorphous Compound I according to claim 1, wherein Compound I is 100% amorphous.

3. Substantially crystalline Compound I methanol solvate (wet).

4. The substantially crystalline Compound I methanol solvate (wet) according to claim 3, wherein Compound I methanol solvate (wet) is 100% crystalline.

5. The substantially crystalline Compound I methanol solvate (wet) according to claim 3, characterized by an X-ray powder diffractogram having a signal at one or more of 25.5±0.2 degrees two-theta, 21.0±0.2 degrees two-theta, 20.5±0.2 degrees two-theta, 19.0±0.2 degrees two-theta, 18.9±0.2 degrees two-theta, 18.6±0.2 degrees two-theta, 16.9±0.2 degrees two-theta, 15.0±0.2 degrees two-theta, 14.6±0.2 degrees two-theta, and 8.4±0.2 degrees two-theta.

6. The substantially crystalline Compound I methanol solvate (wet) according to claim 3, characterized by an X-ray powder diffractogram substantially similar to FIG. 3.

7. The substantially crystalline Compound I methanol solvate (wet) claim 3, characterized by a monoclinic crystal system, P2₁ space group, and the following unit cell dimensions measured at 100 K on a Rigaku diffractometer equipped with Cu Ka radiation (λ=1.54178 Å):

| | | | |
|---|---|---|---|
| a | 6.7 ± 0.1 Å | α | 90° |
| b | 41.5 ± 0.1 Å | β | 92.1 ± 0.1° |
| c | 14.2 ± 0.1 Å | γ | 90°. |

8. The substantially crystalline Compound I methanol solvate (wet) according to claim 3, characterized by a ¹³C SSNMR spectrum having one or more signals selected from 145.6±0.2 ppm, 132.5±0.2 ppm, 113.1±0.2 ppm, 73.5±0.2 ppm, 55.9±0.2 ppm, 35.2±0.2 ppm, 31.1±0.2 ppm, 30.5±0.2 ppm, 24.8±0.2 ppm, and 19.0±0.2 ppm.

9. The substantially crystalline Compound I methanol solvate (wet) according to claim 3, characterized by a ¹³C SSNMR spectrum substantially similar to FIG. 4.

10. The substantially crystalline Compound I methanol solvate (wet) according to claim 3, characterized by a ¹⁹F MAS having one or more signals selected from −63.9±0.2 ppm, −76.6±0.2 ppm, and −79.7±0.2 ppm.

11. The substantially crystalline Compound I methanol solvate (wet) according to claim 3, characterized by a ¹⁹F MAS substantially similar to FIG. 5.

12. The substantially crystalline Compound I methanol solvate (wet) according to claim 3, prepared by (i) combining Compound I neat Form A, water and methanol in a sealed vial, (ii) heating to 65° C. and stirring until a homogeneous slurry is formed, and (iii) cooling the slurry without stirring and allow to sit at room temperature over 3 days, to yield crystalline Compound I methanol solvate (wet).

13. Substantially crystalline Compound I methanol solvate (dry).

14. The substantially crystalline Compound I methanol solvate (dry) according to claim 13, wherein Compound I methanol solvate (dry) is 100% crystalline.

15. The substantially crystalline Compound I methanol solvate (dry) according to claim 13, characterized by an X-ray powder diffractogram having signals at one or more of 27.2±0.2 degrees two-theta, 26.4±0.2 degrees two-theta, 25.9±0.2 degrees two-theta, 21.4±0.2 degrees two-theta, 19.3±0.2 degrees two-theta, 18.1±0.2 degrees two-theta, 15.4±0.2 degrees two-theta, 14.2±0.2 degrees two-theta, and 7.4±0.2 degrees two-theta.

16. The substantially crystalline Compound I methanol solvate (dry) according to claim 13, characterized by an X-ray powder diffractogram substantially similar to FIG. 6.

17. The substantially crystalline Compound I methanol solvate (dry) according to claim 13, prepared by (i) combining Compound I neat Form A, water and methanol in a sealed vial, (ii) heating to 65° C. and stirring until a homogeneous slurry is formed, (iii) cooling the slurry without stirring and allowing it to sit at room temperature over 3 days, and (iv) drying in a vacuum oven at 60° C. overnight to yield crystalline Compound I methanol solvate (dry).

18. Substantially crystalline Compound I p-toluene sulfonic acid.

19. The substantially crystalline Compound I p-toluene sulfonic acid according to claim 18, wherein Compound I p-toluene sulfonic acid is 100% crystalline.

20. The substantially crystalline Compound I p-toluene sulfonic acid according to claim 18, characterized by an X-ray powder diffractogram having signals at one or more of 5.7±0.2 degrees two-theta, 5.8±0.2 degrees two-theta, 7.4±0.2 degrees two-theta, 10.1±0.2 degrees two-theta, 11.5±0.2 degrees two-theta, 11.9±0.2 degrees two-theta, 14.9±0.2 degrees two-theta, 15.9±0.2 degrees two-theta, 16.2±0.2 degrees two-theta, 18.3±0.2 degrees two-theta, 20.4±0.2 degrees two-theta, 21.0±0.2 degrees two-theta, 21.6±0.2 degrees two-theta, 22.8±0.2 degrees two-theta, and 23.2±0.2 degrees two-theta.

21. The substantially crystalline Compound I p-toluene sulfonic acid according to claim 18, characterized by an X-ray powder diffractogram substantially similar to FIG. 9.

22. The substantially crystalline Compound I p-toluene sulfonic acid according to claim 18, characterized by a ¹³C SSNMR spectrum having one or more signals selected from 141.0±0.2 ppm, 126.7±0.2 ppm, 57.0±0.2 ppm, 31.0±0.2 ppm, 25.0±0.2 ppm, 23.0±0.2 ppm, and 19.5±0.2 ppm.

23. The substantially crystalline Compound I p-toluene sulfonic acid according to claim 18, characterized by a ¹³C SSNMR spectrum substantially similar to FIG. 11.

24. The substantially crystalline Compound I p-toluene sulfonic acid according to claim 18, characterized as having a ¹⁹F MAS with one or more signals selected from −62.5±0.2 ppm, −64.2±0.2 ppm, −64.6±0.2 ppm, −65.4±0.2 ppm, −66.1±0.2 ppm, −77.4±0.2 ppm, −78.1±0.2 ppm, −79.7±0.2 ppm, −80.1±0.2 ppm.

25. The substantially crystalline Compound I p-toluene sulfonic acid according to claim 18, characterized by a ¹⁹F MAS substantially similar to FIG. 12.

26. The substantially crystalline Compound I methanol solvate (wet) according to claim 18, prepared by (i) adding p-toluene sulfonic acid to Compound I neat Form A in a milling ball tube, (ii) adding methanol and water (60:40 v/v), (iii) ball mill at 7500 rpm for 3 cycles of 60 seconds with 10 second pauses, and (iv) drying the resulting material in a vacuum dry oven at 40° C., to yield crystalline Compound I p-toluene sulfonic acid.

27. A pharmaceutical composition comprising Compound I according to any one of claims 1-26 and a pharmaceutically acceptable carrier

28. The pharmaceutical composition according to claim 27 further comprising one or more additional thereapeutic agents.

29. The pharmaceutical composition according to claim 28, wherein the pharmaceutical composition comprises one or more additional CFTR modulating compounds.

30. The pharmaceutical composition according to claim 28, wherein the pharmaceutical composition comprises one or more compounds selected from Compound II, Compound III, Compound III-d, Compound IV, Compound V, Compound VI, Compound VII, Compound VIII, Compound IX, Compound X, and pharmaceutically acceptable salts and deuterated derivatives thereof.

31. The Compound I according to any one of claims 1-26, or the pharmaceutical composition according to any one of claims 27-30, for use in the treatment of cystic fibrosis.

32. Use of the Compound I according to any one of claims 1-26, or the pharmaceutical composition according to any one of claims 27-30, in the manufacture of a medicament for the treatment of cystic fibrosis.

33. A method of treating cystic fibrosis comprising administering the Compound I according to any one of claims 1-26, or the pharmaceutical composition according to any one of claims 27-30, to a subject in need thereof.

34. A method for preparing Compound I: 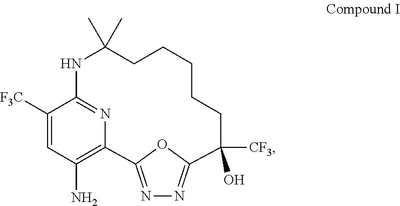 [SMILES: CC1(C)CCCCC[C@](O)(C(F)(F)F)C2=NN=C(O2)c2nc(c(C(F)(F)F)cc2N)N1]
or a stereoisomer of Compound I, or a deuterated derivative of Compound I or a stereoisomer thereof, or pharmaceutically acceptable salts of any of the foregoing, wherein the method comprises converting a compound of Formula I: 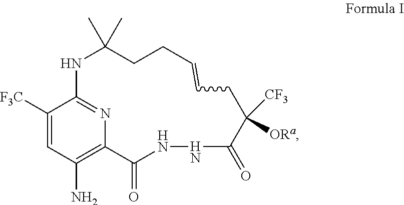
or a stereoisomer of the compound of Formula I, or a deuterated derivative of the compound of Formula I or a stereoisomer thereof, or salts of any of the foregoing, into Compound I, or a stereoisomer thereof, or a deuterated derivative of Compound I or a stereoisomer thereof, or salts of any of the foregoing, wherein R^{a} is selected from alcohol protecting groups.

35. A method for preparing Compound I: 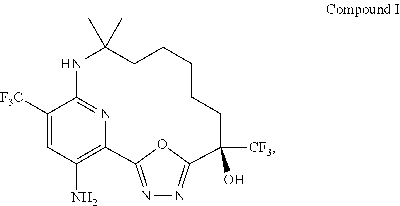 [SMILES: CC1(C)CCCCC[C@](O)(C(F)(F)F)C2=NN=C(O2)c2nc(c(C(F)(F)F)cc2N)N1]
or a stereoisomer of Compound I, or a deuterated derivative of Compound I or a stereoisomer thereof, or pharmaceutically acceptable salts of any of the foregoing, wherein the method comprises converting a compound of Formula II: 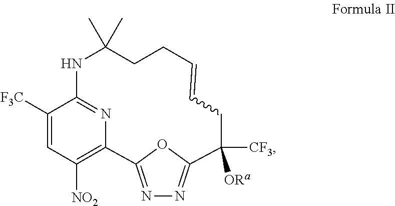
or a stereoisomer of the compound of Formula II, or a deuterated derivative of the compound of Formula II or a stereoisomer thereof, or salts of any of the foregoing, into Compound I, or a stereoisomer thereof, or a deuterated derivative of Compound I or a stereoisomer thereof, or salts of any of the foregoing, wherein R^{a} is selected from alcohol protecting groups, and with the proviso that R^{a} is not benzyl (Bn).

36. The method according to claim 34 or 35, wherein converting the compound of Formula II, or a stereoisomer of the compound of Formula II, or a deuterated derivative of the compound of Formula II or a stereoisomer thereof, or salts of any of the foregoing, into Compound I, or a stereoisomer thereof, or a deuterated derivative of Compound I or a stereoisomer thereof, or salts of any of the foregoing, is performed in the presence of reducing reaction conditions.

37. The method according to any one of claims 34 to 36, wherein the method comprises converting the compound of Formula I: 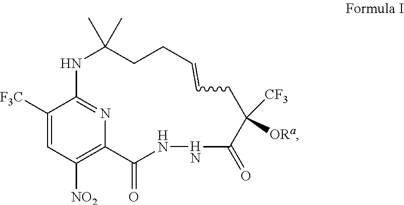
or a stereoisomer of the compound of Formula I, or a deuterated derivative of the compound of Formula I or a stereoisomer thereof, or salts of any of the foregoing, into the compound of Formula II, or a stereoisomer thereof, or a deuterated derivative of the compound of Formula II or a stereoisomer thereof, or salts of any of the foregoing, wherein R^{a} is selected from alcohol protecting groups.

38. The method according to claim 37, wherein converting the compound of Formula I, or a stereoisomer of the compound of Formula I, or a deuterated derivative of the compound of Formula I or a stereoisomer thereof, or salts of any of the foregoing, into the compound of Formula II, or a stereoisomer thereof, or a deuterated derivative of the compound of Formula II or a stereoisomer thereof, or salts of any of the foregoing, is performed in the presence of a dehydrating reagent and a base.

39. The method according to any one of claims 34 to 38, wherein the method comprises converting a compound of Formula III: 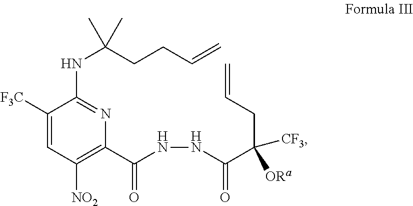
or a stereoisomer of the compound of Formula III, or a deuterated derivative of the compound of Formula III or a stereoisomer thereof, or salts of any of the foregoing, into the compound of Formula I, or a stereoisomer thereof, or a deuterated derivative of the compound of Formula I or a stereoisomer thereof, or salts of any of the foregoing, wherein R^{a} is selected from alcohol protecting groups.

40. The method according to claim 39, wherein converting the compound of Formula III, or a stereoisomer of the compound of Formula III, or a deuterated derivative of the compound of Formula III or a stereoisomer thereof, or salts of any of the foregoing, into the compound of Formula I, or a stereoisomer thereof, or a deuterated derivative of the compound of Formula I or a stereoisomer thereof, or salts of any of the foregoing, is performed in the presence of a ruthenium catalyst.

41. The method according to any one of claims 39 or 40, wherein the method comprises reacting Compound 2: 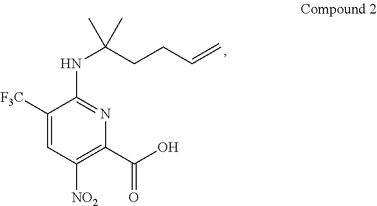 [SMILES: C=CCCC(C)(C)Nc1nc(C(=O)O)c([N+](=O)[O-])cc1C(F)(F)F]
or a deuterated derivative of Compound 2, or a salt of any of the foregoing, with a compound of Formula IV: 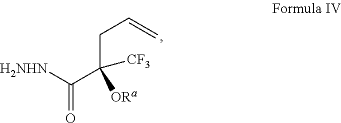
or a stereoisomer of the compound of Formula IV, or a deuterated derivative of the compound of Formula IV or a stereoisomer thereof, or salts of any of the foregoing, to produce the compound of Formula III, or a stereoisomer thereof, or a deuterated derivative of the compound of Formula III or a stereoisomer thereof, or salts of any of the foregoing, wherein R^{a} is selected from alcohol protecting groups.

42. The method according to claim 41, wherein reacting Compound 2, or a deuterated derivative of Compound 2, or a salt of any of the foregoing, with a compound of Formula IV, or a stereoisomer of the compound of Formula IV, or a deuterated derivative of the compound of Formula IV or a stereoisomer thereof, or salts of any of the foregoing, is performed in the presence of a peptide coupling agent and a base.

43. The method according to claim 41 or 42, wherein the method comprises converting a compound of Formula V: 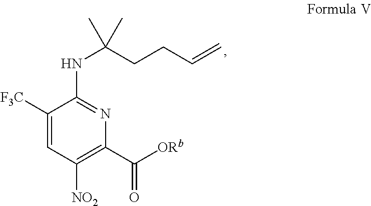
or a deuterated derivative of the compound of Formula V, or a salt of any of the foregoing, into Compound 2, or a deuterated derivative of Compound 2, or a salt of any of the foregoing, wherein R^{b} is selected from methyl (Me), ethyl (Et), n-propyl (n-Pr), isopropyl (i-Pr), and tert-butyl (t-Bu).

44. The method according to claim 43, wherein converting the compound of Formula V, or a deuterated derivative of the compound of Formula V, or a salt of any of the foregoing, into Compound 2, or a deuterated derivative of Compound 2, or a salt of any of the foregoing, is performed in the presence of an aqueous hydroxide base.

45. The method according to 43 or 44, wherein the method comprises reacting a compound of Formula VI: 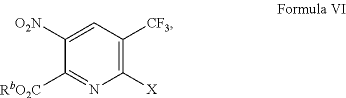
or a deuterated derivative of a compound of Formula VI, or a salt of any of the foregoing, with Compound 3: 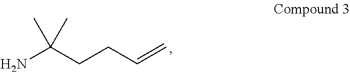
or a deuterated derivative of a compound of Formula VI, or a salt of any of the foregoing, to produce the compound of Formula V, or a deuterated derivative of the compound of Formula V, or a salt of any of the foregoing,
wherein:
R^{b} is selected from methyl (Me), ethyl (Et), n-propyl (n-Pr), isopropyl (i-Pr), and tert-butyl (t-Bu); and
X is selected from Cl and Br.

46. The method according to claim 45, wherein reacting the compound of Formula VI, or a deuterated derivative of the compound of Formula IV, or a salt of any of the foregoing, with compound 3, or a deuterated derivative of Compound 3, or a salt of any of the foregoing, is performed in the presence of a base.

47. The method according to claim 45 or 46, wherein the method comprises converting a compound of Formula VII: 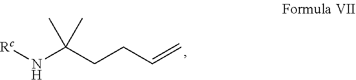
or a deuterated derivative of the compound of Formula VII, or a salt of any of the foregoing, into Compound 3, or a deuterated derivative of Compound 3, or a salt of any of the foregoing, wherein R^{c} is selected from amine protecting groups.

48. The method according to claim 47, wherein converting the compound of Formula VII, or a deuterated derivative of the compound of Formula VII, or a salt of any of the foregoing, into Compound 3, or a deuterated derivative of Compound 3, or a salt of any of the foregoing, is performed in the presence of a protic acid.

49. The method according to claim 47 or 48, wherein the method comprises converting a compound of Formula VIII: 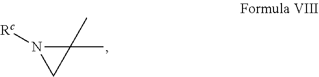
or a deuterated derivative of the compound of Formula VIII, into the compound of Formula VII, or a deuterated derivative of the compound of Formula VII, or a salt of any of the foregoing, wherein R^{c} is selected from amine protecting groups.

50. The method according to claim 49, wherein converting the compound of Formula VIII, or a deuterated derivative of the compound of Formula VIII, into the compound of Formula VII, or a deuterated derivative of the compound of Formula VII, or a salt of any of the foregoing, is performed in the presence of an allylmagnesium halide and a copper(I) halide.

51. The method according to claim 49 or 50, wherein the method comprises converting a compound of Formula IX: 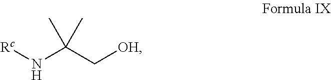
or a deuterated derivative of the compound of Formula IX or a salt of any of the foregoing, into the compound of Formula VIII, or a deuterated derivative of the compound of Formula VIII, or a salt of any of the foregoing, wherein R^{c} is selected from amine protecting groups.

52. The method according to claim 51, wherein converting the compound of Formula IX, or a deuterated derivative of the compound of Formula IX, or a salt of any of the foregoing, into the compound of Formula VIII, or a deuterated derivative of the compound of Formula VIII, or a salt of any of the foregoing, is performed in the presence of a sulfonyl halide and a base.

53. The method according to claim 45 or 46, wherein the method comprises converting a compound of Formula X: 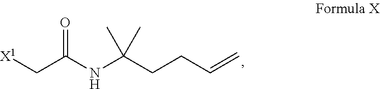
or a deuterated derivative of the compound of Formula X, or a salt of any of the foregoing, into Compound 3, or a deuterated derivative of Compound 3, or a salt of any of the foregoing, wherein X¹ is selected from F, Cl, and Br.

54. The method according to claim 53, wherein converting the compound of Formula X, or a deuterated derivative of the compound of Formula X, or a salt of any of the foregoing, into Compound 3, or a deuterated derivative of Compound 3, or a salt of any of the foregoing, is performed in the presence of thiourea and a protic acid.

55. The method according to claim 53 or 54, wherein the method comprises converting hex-5-en-2-one: 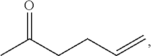
or a deuterated derivative of hex-5-en-2-one, or a salt of any of the foregoing, into the compound of Formula X, or a deuterated derivative of the compound of Formula X, or a salt of any of the foregoing, wherein X¹ is selected from F, Cl, and Br.

56. The method according to claim 55, wherein the method for converting hex-5-en-2-one, or a deuterated derivative of hex-5-en-2-one, or a salt of any of the foregoing, into the compound of Formula X, or a deuterated derivative of the compound of Formula X, or a salt of any of the foregoing, comprises:
(i) reacting hex-5-en-2-one, or a deuterated derivative of hex-5-en-2-one, or a salt of any of the foregoing, with a methylmagnesium halide, or a deuterated derivative of the methylmagnesium halide, or a salt of any of the foregoing; and
(ii) reacting the product of step (i) with a 2-haloacetonitrile or a deuterated derivative of the 2-haloacetonitrile
to produce a compound of Formula X, or a salt of any of the foregoing, wherein X¹ is selected from F, Cl, and Br.

57. A method for preparing Compound I: 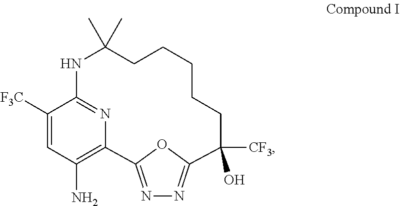 [SMILES: CC1(C)CCCCC[C@](O)(C(F)(F)F)C2=NN=C(O2)c2nc(c(C(F)(F)F)cc2N)N1]
or a stereoisomer of Compound I, or a deuterated derivative of Compound I or a stereoisomer thereof, or pharmaceutically acceptable salts of any of the foregoing, wherein the method comprises converting a compound of Formula I, Formula II, or Formula III: 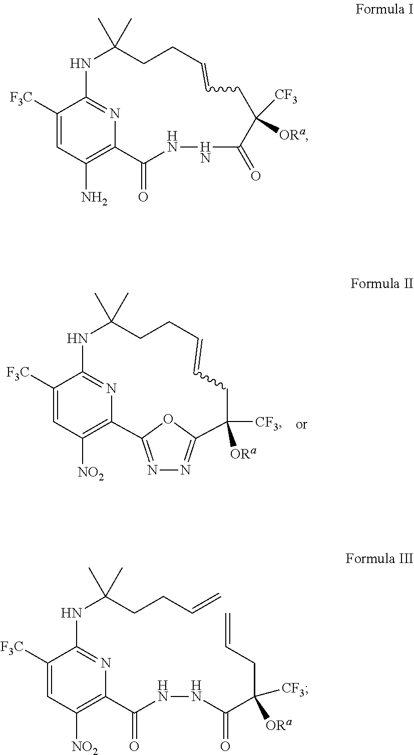
or a stereoisomer of the compound, or a deuterated derivative of the compound or a stereoisomer thereof, or a salt of any of the foregoing, into Compound I, or a stereoisomer of Compound I, or a deuterated derivative of Compound I or a stereoisomer thereof, or a pharmaceutically acceptable salt of any of the foregoing, wherein:
R^{a} is selected from alcohol protecting groups; and
the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (E/Z mixture); and pharmaceutically acceptable salts thereof.

58. A compound selected from: 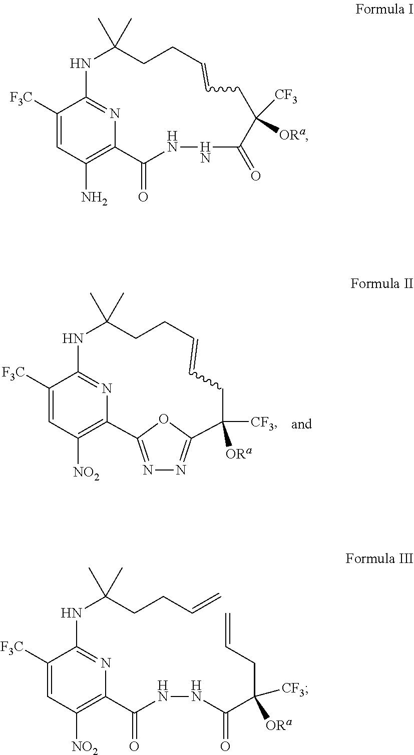
or a stereoisomer of the compound, or a deuterated derivative of the compound or a stereoisomer thereof, or a salt of any of the foregoing,
wherein:
R^{a} is selected from alcohol protecting groups; and
the compound of Formula I, Formula II, or Formula III is not a compound selected from N′-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide; (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E Z mixture); N′-[(2R)-2-benzyloxy-2-(trifluoromethyl)hex-5-enoyl]-6-[1,1-bis(trideuteriomethyl)but-3-enylamino]-3-nitro-5-(trifluoromethyl)pyridine-2-carbohydrazide; (6R)-6-benzyloxy-17-nitro-12,12-bis(trideuteriomethyl)-6,15-bis(trifluoromethyl)-19-oxa-3,4,13,18-tetrazatricyclo[12.3.1.12,5]nonadeca-1(18),2,4,9,14,16-hexaene (E/Z mixture);
and pharmaceutically acceptable salts thereof.

59. A compound selected from: 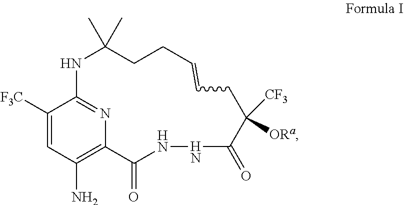
or a stereoisomer of the compound, or a deuterated derivative of the compound or a stereoisomer thereof, or a salt of any of the foregoing, wherein R^{a} is selected from alcohol protecting groups.

60. A compound having the following formula: 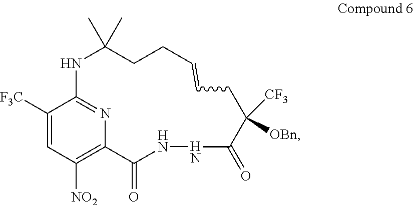 [SMILES: CC1(C)CCC=CC[C@](OCc2ccccc2)(C(F)(F)F)C(=O)NNC(=O)c2nc(c(C(F)(F)F)cc2[N+](=O)[O-])N1]
or a stereoisomer of the compound, or a deuterated derivative of the compound or a stereoisomer thereof, or a salt of any of the foregoing.

61. A method for preparing Compound I: 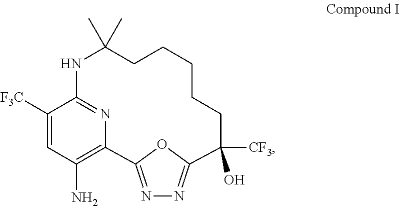 [SMILES: CC1(C)CCCCC[C@](O)(C(F)(F)F)C2=NN=C(O2)c2nc(c(C(F)(F)F)cc2N)N1]
or a stereoisomer of Compound I, or a deuterated derivative of Compound I or a stereoisomer thereof, or pharmaceutically acceptable salts of any of the foregoing, wherein the method comprises converting a compound of Formula II: 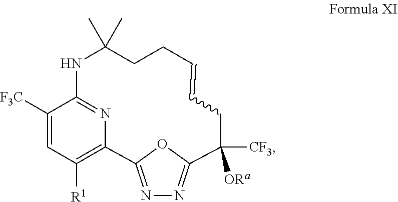
or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing,
wherein:
R^{a} is selected from alcohol protecting groups, and
R¹ is selected from —N(Boc)₂, —NHBoc, —N(Phth), —NH(Cbz), and —NO₂.

62. The method according to claim 62, wherein converting the compound of Formula XI, or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing, comprises a reaction that is performed in the presence of reducing reaction conditions.

63. The method according to claim 62 or 63, wherein converting the compound of Formula XI, or a stereoisomer of the compound of Formula XI, or a deuterated derivative of the compound of Formula XI or a stereoisomer thereof, or salts of any of the foregoing, into Compound I, or a stereoisomer thereof, or a deuterated derivative of Compound I or a stereoisomer thereof, or salts of any of the foregoing, further comprises a reaction that is performed in the presence of an acid.

64. The method according to any one of claims 61 to 63, wherein the method comprises converting the compound of Formula XII: 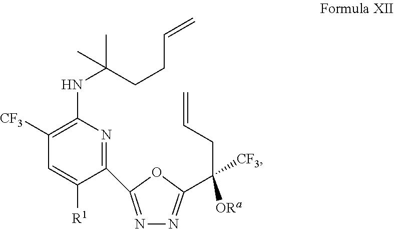
or a stereoisomer of the compound of Formula XII, or a deuterated derivative of the compound of Formula XII or a stereoisomer thereof, or salts of any of the foregoing, into the compound of Formula XI, or a stereoisomer thereof, or a deuterated derivative of the compound of Formula XI or a stereoisomer thereof, or salts of any of the foregoing, wherein:
R^{a} is selected from alcohol protecting groups, and
R¹ is selected from —N(Boc)₂, —NHBoc, —N(Phth), —NH(Cbz), and —NO₂.

65. The method according to claim 64, wherein converting the compound of Formula XII, or a stereoisomer of the compound of Formula XII, or a deuterated derivative of the compound of Formula XII or a stereoisomer thereof, or salts of any of the foregoing, into the compound of Formula XI, or a stereoisomer thereof, or a deuterated derivative of the compound of Formula XI or a stereoisomer thereof, or salts of any of the foregoing, comprises a reaction that is performed in the presence of a ruthenium catalyst.

66. The method according to claim 64 or 65, wherein the method comprises reacting Formula XIII: 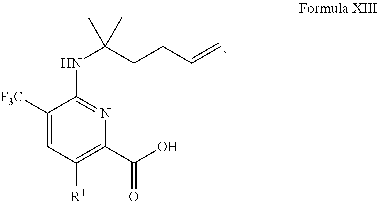
or a deuterated derivative of Compound 2, or a salt of any of the foregoing, with a compound of Formula IV: 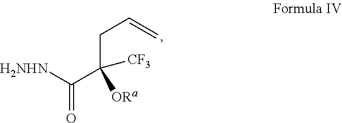
or a stereoisomer of the compound of Formula IV, or a deuterated derivative of the compound of Formula IV or a stereoisomer thereof, or salts of any of the foregoing, to produce the compound of Formula XII, or a stereoisomer thereof, or a deuterated derivative of the compound of Formula XII or a stereoisomer thereof, or salts of any of the foregoing, wherein:
R^{a} is selected from alcohol protecting groups, and
R¹ is selected from —N(Boc)₂, —NHBoc, —N(Phth), —NH(Cbz), and —NO₂.

67. The method according to claim 66, wherein reacting Formula XIII, or a deuterated derivative of Formula XIII, or a salt of any of the foregoing, with a compound of Formula IV, or a stereoisomer of the compound of Formula IV, or a deuterated derivative of the compound of Formula IV or a stereoisomer thereof, or salts of any of the foregoing, is performed in the presence of a peptide coupling agent and a base.
